(19) United States
(12) Patent Application Publication
(10) Pub. No.: US 2019/0128896 A1
(43) Pub. Date: May 2, 2019

(54) METHODS AND SYSTEMS FOR SELECTIVE QUANTITATION AND DETECTION OF ALLERGENS

(71) Applicant: Dow AgroSciences LLC, Indianapolis, IN (US)

(72) Inventors: Trent James Oman, Carmel, IN (US); Barry William Shafer, Cicero, IN (US); Ryan Christopher Hill, Fishers, IN (US); Guomin Shan, Carmel, IN (US)

(73) Assignee: Dow AgroSciences LLC, Indianapolis, IN (US)

(21) Appl. No.: 15/431,487

(22) PCT Filed: Aug. 11, 2015
(86) PCT No.: PCT/US2015/044697

§ 371 Date: Feb. 13, 2017

Related U.S. Application Data
(60) Provisional application No. 62/036,032, filed on Aug. 11, 2014.
(60) Provisional application No. 62/036,024, filed on Aug. 11, 2014.
(60) Provisional application No. 62/036,016, filed on Aug. 11, 2014.
(60) Provisional application No. 62/036,007, filed on Aug. 11, 2014.
(60) Provisional application No. 62/035,997, filed on Aug. 11, 2014.
(60) Provisional application No. 62/035,981, filed on Aug. 11, 2014.
(60) Provisional application No. 62/035,968, filed on Aug. 11, 2014.

(51) Int. Cl. G01N 33/68; G01N 30/72

(52) U.S. Cl. CPC G01N 33/6848; G01N 2030/8831; G01N 2800/24; G01N 30/7233

(57) ABSTRACT

The invention relates to methods and systems taking advantage of bioinformatic investigations to identify candidate signature peptides for quantitative multiplex analysis of complex protein samples from plants, plant parts, and/or food products using mass spectroscopy. Provided are use and methods for selecting candidate signature peptides for quantitation using a bioinformatic approach. Also provided are systems comprising a chromatography and mass spectrometry for using selected signature peptides.

### CROSS-REFERENCE TO RELATED APPLICATIONS

This is a national phase entry under 35 U.S.C. § 371 of international Patent Application PCT/US2015/044697, filed Aug. 11, 2015; which claims benefit of United States Provisional Applications: Ser. No. 62/035,968, filed Aug. 11, 2014; Ser. No. 62/035,981, filed Aug. 11, 2014; Ser. No. 62/035,997, filed Aug. 11, 2014; Ser. No. 62/036,007, filed Aug. 11, 2014; Ser. No. 62/036,016, tiled Aug. 11, 2014; Ser. No. 62/036,024, filed Aug. 11, 2014; Ser. No. 62/036,032, filed Aug. 11, 2014.

### BACKGROUND

The current methods for analysis of gene expression in plants that are preferred in the art include DNA-based techniques (for example PCR and/or RT-PCR); the use of reporter genes; Southern blotting; and immunochemistry. All of these methodologies suffer from various shortcomings. Detection of known and potential allergens in plants, plant parts, and/or food products is an important subject for public safety.

Although mass spectrometry has been disclosed previously, existing approaches are limited without selected and sensitive quantitation. There remains a need for a high-throughput method for selected and sensitive quantitation of known and/or potential allergens in plant, plant parts, and/or food products.

### BRIEF SUMMARY

The invention relates to methods and systems taking advantage of bioinformatic investigations to identify candidate signature peptides for quantitative multiplex analysis of complex protein samples from plants, plant parts, and/or food products using mass spectrometry. Provided are use and methods for selecting candidate signature peptides for quantitation using a bioinformatic approach. Also provided are systems comprising a chromatography and mass spectrometry for using selected signature peptides.

In one aspect, provided is a method of selecting candidate signature peptide for quantitation of known allergen and potential allergens from a plant-based sample. The method comprises:
(a) identifying potential allergens based on homology to at least one known allergen protein sequence;
(b) performing sequence alignment of the at least one known allergen and potential allergens identified in step (a);
(c) selecting a consensus sequence or representative sequence based on the sequence alignment;
(d) determining a plural of candidate signature peptides based on conservative regions or domains from the sequence alignment and in silico digestion data of the consensus sequence or representative sequence selected in Step (c); and
(e) quantitating the amount of the at least one known allergen and potential allergens in the plant-based sample based on measurements of the signature peptides.

In one embodiment, the quantitating step uses a column chromatography and mass spectrometry. In another embodiment, the quantitating step comprises measuring the plural of candidate signature peptides using high resolution accurate mass spectrometry (HRAM MS). In another embodiment, the quantitating step comprises calculating corresponding peak heights or peak areas of the candidate signature peptides from mass spectrometry. In another embodiment, the quantitating step comprises comparing data from high fragmentation mode and low fragmentation mode from mass spectrometry.

In one embodiment, the at least one known allergen comprises at least one allergen selected from the group consisting of Gly m 1, Gly m 3, Gly m 4, Gly m 5 (beta-conglycinin), Gly m 6 (Glycinin) G1, Gly m 6 (Glycinin) G2, Gly m 6 (Glycinin) G3, Gly m 6 (Glycinin) G4, Gly m 6 (Glycinin) precursor, Kunitz trypsin inhibitor 1, Kunitz trypsin inhibitor 3, Gly m Bd 28 K, Gly m Bd 30 K, Gly m 8 (2S albumin), Lectin, and lipoxygenase. In another embodiment, the at least one known allergen comprises Gly m Bd 28 K, Gly m Bd 30 K, Kunitz trypsin inhibitor 1, Kunitz trypsin inhibitor 3, Gly m 8 (2S albumin), Lectin, or lipoxygenase.

In another embodiment, the consensus sequence or representative sequence of step (c) comprises SEQ ID NO: 21, 28, 29, 30, 31, 32, or 33 for Gly m Bd 28 K. In a further embodiment, the consensus sequence or representative sequence of step (c) comprises SEQ ID NO: 21 or 28 for Gly m Bd 28 K. In another embodiment, the consensus sequence or representative sequence of step (c) comprises SEQ ID NO: 22, 43, or 44 for Gly m Bd 30 K. In a further embodiment, the consensus sequence or representative sequence of step (c) comprises SEQ ID NO: 22 or 43 for Gly m Bd 30 K. In another embodiment, the consensus sequence or representative sequence of step (c) comprises SEQ ID NO: 23, 52, 53, or 54 for Kunitz trypsin inhibitor 1. In a further embodiment, the consensus sequence or representative sequence of step (c) comprises SEQ ID NO: 23 or 52 for Kunitz trypsin inhibitor 1. In another embodiment, the consensus sequence or representative sequence of step (c) comprises SEQ ID NO: 24, 61, 62, or 63 for Kunitz trypsin inhibitor 3. In a further embodiment, the consensus sequence or representative sequence of step (c) comprises SEQ ID NO: 24 or 61 for Kunitz trypsin inhibitor 3. In another embodiment, the consensus sequence or representative sequence of step (c) comprises SEQ ID NO: 25, 72, 73, or 74 for Gly m 8 (2S albumin). In a further embodiment, the consensus sequence or representative sequence of step (c) comprises SEQ ID NO: 25 for Gly m 8 (2S albumin). In another embodiment, the consensus sequence or representative sequence of step (c) comprises SEQ ID NO: 26, 82, 83, 84, 85, 86, 87, 88, 89, or 90 for Lectin. In a further embodiment, the consensus sequence or representative sequence of step (c) comprises SEQ ID NO: 26 or 82 for Lectin. In another embodiment, the consensus sequence or representative sequence of step (c) comprises SEQ ID NO: 27, 95, 96, 97, 98, 99, 100, 101, 102, 103, or 104 for lipoxygenase. In a further embodiment, the consensus sequence or representative sequence of step (c) comprises SEQ ID NO: 27 or 95 for lipoxygenase.

In one embodiment, the potential allergens comprise at least one sequence selected from SEQ ID NOs: 21 and 29-33 for Gly m Bd 28 K. In another embodiment, the potential allergens comprise at least one sequence selected from SEQ ID NOs: 22 and 43-44 for Gly m Bd 30 K. In another embodiment, the potential allergens comprise at least one sequence selected from SEQ ID NOs: 23 and 53-54 for Kunitz trypsin inhibitor 1. In another embodiment, the potential allergens comprise at least one sequence selected from SEQ ID NOs: 24 and 62-63 for Kunitz trypsin inhibitor 3. In another embodiment, the potential allergens comprise at least one sequence selected from SEQ ID NOs: 25 and 72-74 for Gly m 8 (2S albumin). In another embodiment, the potential allergens comprise at least one sequence selected from SEQ ID NOs: 26 and 83-90 for Lectin. In another embodiment, the potential allergens comprise at least one sequence selected from SEQ ID NOs: 27 and 96-104 for lipoxygenase.

In another embodiment, the candidate signature peptides comprise at least one sequence selected from SEQ ID NOs: 9 and 34-42 for Gly m Bd 28 K. In another embodiment, the candidate signature peptides comprise SEQ ID NOs: 34-42 for Gly m Bd 28 K. In another embodiment, the candidate signature peptides comprise at least one sequence selected from SEQ ID NOs: 10 and 45-51 for Gly m Bd 30 K. In another embodiment, the candidate signature peptides comprise SEQ ID NOs: 45-51 for Gly m Bd 30 K. In another embodiment, the candidate signature peptides comprise at least one sequence selected from SEQ ID NOs: 7 and 55-60 for Kunitz trypsin inhibitor 1. In another embodiment, the candidate signature peptides comprise SEQ ID NOs: 55-60 for Kunitz trypsin inhibitor 1. In another embodiment, the candidate signature peptides comprise at least one sequence selected from SEQ ID NOs: 8 and 64-71 for Kunitz trypsin inhibitor 3. In another embodiment, the candidate signature peptides comprise SEQ ID NOs: 64-71 for Kunitz trypsin inhibitor 3. In another embodiment, the candidate signature peptides comprise at least one sequence selected from SEQ ID NOs: 11 and 75-81 for Gly m 8 (2S albumin). In another embodiment, the candidate signature peptides comprise SEQ ID NOs: 75-81 for Gly m 8 (2S albumin). In another embodiment, the candidate signature peptides comprise at least one sequence selected from SEQ ID NOs: 91-94 for Lectin. In another embodiment, the candidate signature peptides comprise SEQ ID NOs: 91-94 for Lectin. In another embodiment, the candidate signature peptides comprise at least one sequence selected from SEQ ID NOs: 105-120 for lipoxygenase. In another embodiment, the candidate signature peptides comprise SEQ ID NOs: 105-120 for lipoxygenase. In another embodiment, the plant-based sample comprises a soybean seed or part of a soybean seed.

In another aspect, provided is a system for quantitating one or more protein of interest with known amino acid sequence in a plant-based sample. The system comprises:
(a) a high-throughput means for extracting proteins from a plant-based sample;
(b) a process module for digesting extracted proteins with at least one protease;
(c) a separation module for separating peptides in a single step;
(d) a selection module for selecting a plural of signature peptides for at least one known allergen and potential allergens; and
(e) a mass spectrometry for measuring the plural of signature peptides.

In one embodiment, the separation module comprises a column chromatography. In a further embodiment, the column chromatography comprises a liquid column chromatography. In another embodiment, the mass spectrometry comprises a high resolution accurate mass spectrometry (HRAM MS). In another embodiment, the selection module uses a method provided herein.

In one embodiment, the one or more protein of interest with known amino acid sequence in a plant-based sample comprises potential allergens. In another embodiment, the potential allergens comprise at least one sequence selected from SEQ ID NOs: 21 and 29-33 for Gly m Bd 28 K. In another embodiment, the potential allergens comprise at least one sequence selected from SEQ ID NOs: 22 and 43-44 for Gly m Bd 30 K. In another embodiment, the potential allergens comprise at least one sequence selected from SEQ ID NOs: 23 and 53-54 for Kunitz trypsin inhibitor 1. In another embodiment, the potential allergens comprise at least one sequence selected from SEQ ID NOs: 24 and 62-63 for Kunitz trypsin inhibitor 3. In another embodiment, the potential allergens comprise at least one sequence selected from SEQ ID NOs: 25 and 72-74 for Gly m 8 (2S albumin). In another embodiment, the potential allergens comprise at least one sequence selected from SEQ ID NOs: 26 and 83-90 for Lectin. In another embodiment, the potential allergens comprise at least one sequence selected from SEQ ID NOs: 27 and 96-104 for lipoxygenase.

In another embodiment, the signature peptides comprise at least one sequence selected from SEQ ID NOs: 9 and 34-42 for Gly m Bd 28 K. In another embodiment, the signature peptides comprise SEQ ID NOs: 34-42 for Gly m Bd 28 K. In another embodiment, the signature peptides comprise at least one sequence selected from SEQ ID NOs: 10 and 45-51 for Gly m Bd 30 K. In another embodiment, the signature peptides comprise SEQ ID NOs: 45-51 for Gly m Bd 30 K. In another embodiment, the signature peptides comprise at least one sequence selected from SEQ ID NOs: 7 and 55-60 for Kunitz trypsin inhibitor 1. In another embodiment, the signature peptides comprise SEQ ID NOs: 55-60 for Kunitz trypsin inhibitor 1. In another embodiment, the signature peptides comprise at least one sequence selected from SEQ ID NOs: 8 and 64-71 for Kunitz trypsin inhibitor 3. In another embodiment, the signature peptides comprise SEQ ID NOs: 64-71 for Kunitz trypsin inhibitor 3. In another embodiment, the signature peptides comprise at least one sequence selected from SEQ ID NOs: 11 and 75-81 for Gly m 8 (2S albumin). In another embodiment, the signature peptides comprise SEQ ID NOs: 75-81 for Gly m 8 (2S albumin). In another embodiment, the signature peptides comprise at least one sequence selected from SEQ ID NOs: 91-94 for Lectin. In another embodiment, the signature peptides comprise SEQ ID NOs: 91-94 for Lectin. In another embodiment, the signature peptides comprise at least one sequence selected from SEQ ID NOs: 105-120 for lipoxygenase. In another embodiment, the signature peptides comprise SEQ ID NOs: 105-120 for lipoxygenase. In another embodiment, the plant-based sample comprises a soybean seed or part of a soybean seed.

In another aspect, provided is a high-throughput method of quantitating at least one allergen with known amino acid sequence and homologous potential allergens in a plant-based sample. The method comprises using the system provided herein.

### BRIEF DESCRIPTION OF THE DRAWINGS

FIG. 1 shows a representative analysis work flow for the methods and systems disclosed herein.

FIGS. 2A-2J show representative SRM LC-MS/MS for selected signature peptides SEQ ID NO: 9 NKPQFLAGAASLLR; SEQ ID NO: 34 LGFIYDDELAER; SEQ ID NO: 35 TVVEEIFSK; SEQ ID NO: 36 MMQDQEEDEEEK; SEQ ID NO: 37 NAYGWSK; SEQ ID NO: 38 ALHGGEYPPLSEPDIGVLLVK; SEQ ID NO: 39 QGDVFVVPR; SEQ ID NO: 40 YFPFCQVASR; SEQ ID NO: 41 TLMGPELSAAFGVSEDTLR; SEQ ID NO: 42 SFANDVVMDVF from trypsin digested soybean sample chromatogram for Gly m Bd 28 K.

FIG. 2K shows representative SRM LC-MS/MS Standard Chromatogram—500 ng/mL Synthetic Peptide SEQ ID NO: 9 NKPQFLAGAASLLR natural abundance peptide and heavy isotope labeled peptide transitions.

FIG. 2L shows sequences alignments among Gly m Bd 28 K and potential homologs of Gly m Bd 28 K.

FIGS. 3A-3H show representative SRM LC-MS/MS for selected signature peptides SEQ ID NO: 10 GVITQVK; SEQ ID NO: 45 SILDLDLTK; SEQ ID NO: 46 FTTQK; SEQ ID NO: 47 NNLNYIR; SEQ ID NO: 48 FADITPQEFSK; SEQ ID NO: 49 EQYSCDHPPASWDWR; SEQ ID NO: 50 VTIDGYETLIMSDESTESETEQAFL SA1LEQPISVSIDAK; and SEQ ID NO: 51 NTGNLLGVCGMNYFASYPTK; from trypsin digested soybean sample chromatogram for Gly m Bd 30 K.

FIG. 3I shows representative SRM LC-MS/MS Standard Chromatogram—500 ng/mL Synthetic Peptide SEQ ID NO: 10 GVITQVK natural abundance peptide and heavy isotope labeled peptide transitions.

FIG. 3J shows sequences alignments among Gly m Bd 30 K and potential homologs of Gly m Bd 30 K.

FIGS. 4A-4G show representative SRM LC-MS/MS for selected signature peptides SEQ ID NO: 7 GGGIEVDSTGK; SEQ ID NO: 55 EICPLTVVQSPNELDK; SEQ ID NO: 56 EGLQAVK; SEQ ID NO: 57 LVFCPQQAEDNK; SEQ ID NO: 58 CEDIGIQIDDDGIR; SEQ ID NO: 59 LVLSK; and SEQ ID NO: 60 NKPLVVQFQK from trypsin digested soybean sample chromatogram for Kunitz trypsin inhibitor 1.

FIG. 4H shows representative SRM LC-MS/MS Standard Chromatogram—500 ng/mL Synthetic Peptide SEQ ID NO: 7 GGGIEVDSTGK natural abundance peptide and heavy isotope labeled peptide transitions.

FIG. 4I shows sequences alignments among Kunitz trypsin inhibitor 1 and potential homologs of Kunitz trypsin inhibitor 1.

FIGS. 5A-5I show representative SRM LC-MS/MS for selected signature peptides SEQ ID NO: 8 GIGTIISSPYR; SEQ ID NO: 64 CPLTVVQSR; SEQ ID NO: 65 NELDK; SEQ ID NO: 66 IGENK; SEQ ID NO: 67 DAMDGWFR; SEQ ID 68 LVFCPQQAEDDK; SEQ ID NO: 69 CGDIGISIDHDDGTR; SEQ ID NO: 70 LVVSK; and SEQ ID NO: 71 NKPLVVQFQK from trypsin digested soybean sample chromatogram for Kunitz trypsin inhibitor 3.

FIG. 5J shows representative SRM LC-MS/MS Standard Chromatogram—500 ng/mL Synthetic Peptide SEQ ID NO: 8 GIGTIISSPYR natural abundance peptide and heavy isotope labeled peptide transitions.

FIG. 5K shows sequences alignments among Kunitz trypsin inhibitor 3 and potential homologs of Kunitz trypsin inhibitor 3.

FIGS. 6A-6H show representative SRM LC-MS/MS for selected signature peptides SEQ ID NO: 11 IMENQSEELEEK; SEQ ID NO: 75 WQHQQDSCR; SEQ ID NO: 76 QLQGVNLTPCEK; SEQ ID NO: 77 HIMEK; SEQ ID NO: 78 DEDEEEEGHMQK; SEQ ID NO: 79 CCTEMSELR; SEQ ID NO: 80 ELINLATMCR; and SEQ ID NO: 81 FGPMIQCDLSSDD from trypsin digested soybean sample chromatogram for Gly m 8 (2S albumin).

FIG. 6I shows representative SRM LC-MS/MS Standard Chromatogram—500 ng/mL Synthetic Peptide SEQ ID NO: 11 IMENQSEELEEK natural abundance peptide and heavy isotope labeled peptide transitions.

FIG. 6J shows sequences alignments among Gly m 8 (2S albumin) and potential homologs of Gly m 8.

FIGS. 7A-7D show representative SRM LC-MS/MS for selected signature peptides SEQ ID NO: 91 VFSPNK; SEQ ID NO: 92 ANSTNTVSFTVSK; SEQ ID NO: 93 QQNLIFQGDAAISPSGVLR; and SEQ ID NO: 94 TADGLAFFLAPVGSKPQSK from trypsin digested soybean sample chromatogram for Lectin.

FIG. 7E shows representative SRM LC-MS/MS Standard Chromatogram—500 ng/mL Synthetic Peptide SEQ ID NO: 91 VFSPNK natural abundance peptide and heavy isotope labeled peptide transitions.

FIG. 7F shows sequences alignments among lectin and potential homologs of Lectin.

FIGS. 8A-8P show representative SRM LC-MS/MS for selected signature peptides SEQ ID NO: 105 SSDFLTYGIK; SEQ ID NO: 106 GTVVLMPK; SEQ ID NO: 107 NVLDFNAITSIGK; SEQ ID NO: 108 GGVIDTATGILGQGVSLVGGVIDTATSFLGR; SEQ ID NO: 109 IFFVNDTYLPSATPAPLLK; SEQ ID NO: 110 DENFGHLK; SEQ ID NO: 111 SLSHDVIPLFK; SEQ ID NO: 112 SLYEGGIK; SEQ ID NO: 113 TDGENVLQFPPPHVAK; SEQ ID NO: 114 INSLPTAK; SEQ ID NO: 115 TILFLK; SEQ ID NO: 116 HLSVLHPIYK; SEQ ID NO: 117 QSLINADGIIEK; SEQ ID NO: 118 FIPAEGTPEYDEMVK; SEQ ID NO: 119 ALEAFK; and SEQ ID NO: 120 GIPNSISI from trypsin digested soybean sample chromatogram.

FIG. 8Q shows representative SRM LC-MS/MS Standard Chromatogram—500 ng/mL Synthetic Peptide SEQ ID NO: 105 SSDFLTYGIK natural abundance peptide and heavy isotope labeled peptide transitions.

FIG. 8R shows sequences alignments among lipoxygenase and potential homologs of lipoxygenase.

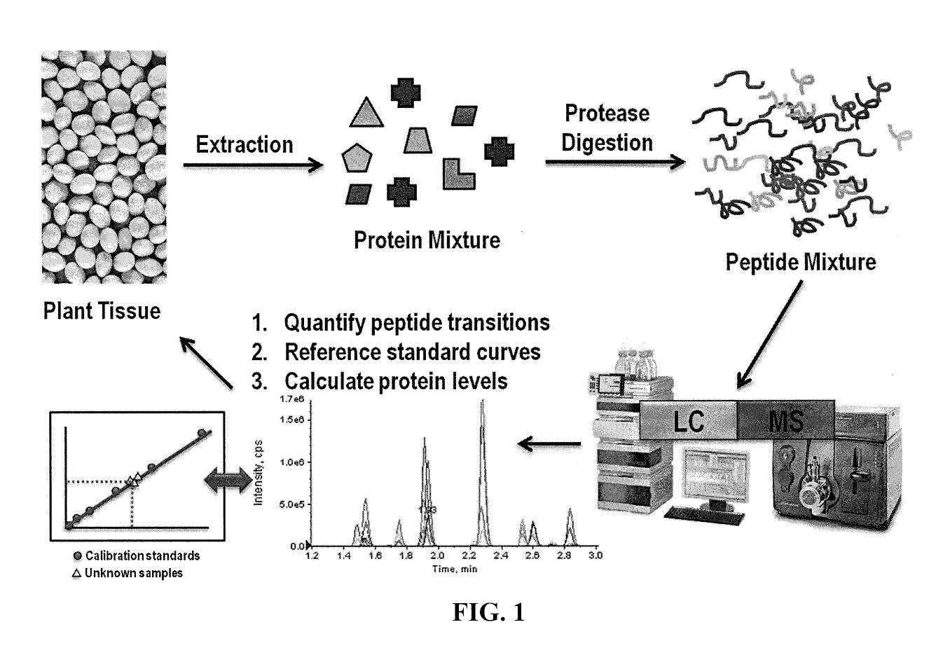

### SEQUENCE LISTING

The nucleic acid sequences listed in the accompanying sequence listing are shown using standard letter abbreviations for nucleotide bases, as defined in 37 C.F.R. § 1.822. The nucleic acid and amino acid sequences listed define molecules (i.e., polynucleotides and polypeptides, respectively) having the nucleotide and amino acid monomers arranged in the manner described. The nucleic acid and amino acid sequences listed also each define a genus of polynucleotides or polypeptides that comprise the nucleotide and amino acid monomers arranged in the manner described. In view of the redundancy of the genetic code, it will be understood that a nucleotide sequence including a coding sequence also describes the genus of polynucleotides encoding the same polypeptide as a polynucleotide consisting of the reference sequence. It will further be understood that an amino acid sequence describes the genus of polynucleotide ORFs encoding that polypeptide.

Only one strand of each nucleic acid sequence is shown, but the complementary strand is understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears). Furthermore, as it is understood in the art that the nucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NO: 1
Exemplary signature peptide for Gly m 1
SYPSNATCPR
SEQ ID NO: 2
Exemplary signature peptide for Gly m 3
YMVIQGEPGAVIR
SEQ ID NO: 3
Exemplary signature peptide for Gly m 5 (beta-conglycinin)
NILEASYDTK
SEQ ID NO: 4
Exemplary signature peptide for Gly m 6 (Glycinin)
G2VTAPAMR
SEQ ID NO: 5
Exemplary signature peptide for Gly m 6 (Glycinin) G3
NNNPFSFLVPPK
SEQ ID NO: 6
Exemplary signature peptide for Gly m 6 (Glycinin) precursor
ADFYNPK
SEQ ID NO: 7
Exemplary signature peptide for Kunitz trypsin inhibitor 1
GGGIEVDSTGK
SEQ ID NO: 8
Exemplary signature peptide for Kunitz trypsin inhibitor 3
GIGTIISSPYR
SEQ ID NO: 9
Exemplary signature peptide for Gly m Bd 28 K
NKPQFLAGAASLLR
SEQ ID NO: 10
Exemplary signature peptide for Gly m Bd 30 K
GVITQVK
SEQ ID NO: 11
Exemplary signature peptide for Gly m 8 (2S albumin)
IMENQSEELEEK
SEQ ID NO: 12
Gly m 1 ABA54898.1 [MW = 12482.64 Da]
MGSKVVASVALLLSINILFISMVSSSSHYDPQPQPSHVTALITRPSCPDLSICLNILGGSLG
TVDDCCALIGGLGDIEAIVCLCIQLRALGILNLNRNLQLILNSCGRSYPSNATCPRT
SEQ ID NO: 13
Gly m 3 CAA11755.1 [MW = 14100.07 Da]
MSWQAYVDDHLLCGIEGNHLTHAAIIQDGSVWLQSTDFPQFKPEEITAIMNDFNEPGS
LAPTGLYLGGTKYMVIQGEPGAVIRGKKGPGGVTVKKTGAALIIGIYDEPMTPGQCNMV
VERLGDYLIDQGY
SEQ ID NO: 14
Gly m 4 P26987 [MW = 16771.81 Da]
MGVFTFEDEINSPVAPATLYKALVTDADNVIPKALDSFKSVENVEGNGGPGTIKKITFLE
DGETKFVLHKIESIDEANLGYSYSVVGGAALPDTAEKITFDSKLVAGPNGGSAGKLTVK
YETKGDAEPNQDELKTGKAKADALFKAIEAYLLAHPDYN
SEQ ID NO: 15
Gly m 5 (beta-conglycinin) 121281 [MW = 70293.13 Da]
MMRARFPLLLLGLVFLASVSVSFGIAYWEKENPKHNKCLQSCNSERDSYRNQACHARC
NLLKVEKEECEEGEIPRPRPRPQHPEREPQQPGEKEEDEDEQPRPIPFPRPQPRQEEEHEQ
REEQEWPRKEEKRGEKGSEEEDEDEDEEQDERQFPFPRPPHQKEERNEEEDEDEEQQRE
SEESEDSELRRHKNKNPFLFGSNRFETLFKNQYGRIRVLQRFNQRSPQLQNLRDYRILEF
NSKPNTLLLPNHADADYLIVILNGTAILSLVNNDDRDSYRLQSGDALRVPSGTTYYVVN
PDNNENLRLITLAIPVNKPGRFESFFLSSTEAQQSYLQGFSRNILEASYDTKFEEINKVLFS
REEGQQQGEQRLQESVIVEISKEQIRALSKRAKSSSRKTISSEDKPFNLRSRDPIYSNKLGK
FFEITPEKNPQLRDLDIFLSIVDMNEGALLLPHFNSKAIVILVINEGDANIELVGLKEQQQE
QQQEEQPLEVRKYRAELSEQDIFVIPAGYPVVVNATSNLNFFAIGINAENNQRNFLAGSQ
DNVISQIPSQVQELAFPGSAQAVEKLLKNQRESYFVDAQPKKKEEGNKGRKGPLSSILRA
FY
SEQ ID NO: 16
Gly m 6 Glycinin G1 121276 [MW = 55706.34 Da]
MAKLVFSLCFLLFSGCCFAFSSREQPQQNECQIQKLNALKPDNRIESEGGLIETWNPNNK
PFQCAGVALSRCTLNRNALRRPSYTNGPQEIYIQQGKGIFGMIYPGCPSTFEEPQQPQQR
GQSSRPQDRHQKIYNFREGDLIAVPTGVAWWMYNNEDTPVVAVSIIDTNSLENQLDQM
PRRFYLAGNQEQEFLKYQQEQGGHQSQKGKHQQEEENEGGSILSGFTLEFLEHAFSVDK
QIAKNLQGENEGEDKGAIVTVKGGLSVIKPPTDEQQQRPQEEEEEEEDEKPQCKGKDKH
CQRPRGSQSKSRRNGIDETICTMRLRHNIGQTSSPDIYNPQAGSVTTATSLDFPALSWLRL
SAEFGSLRKNAMFVPHYNLNANSIIYALNGRALIQVVNCNGERVFDGELQEGRVLIVPQ
NFVVAARSQSDNFEYVSFKTNDTPMIGTLAGANSLLNALPEEVIQHTFNLKSQQARQIK
NNNPFKFLVPPQESQKRAVA
SEQ ID NO: 17
Gly m 6 Glycinin G2 121277 [MW = 54390.76 Da]
MAKLVLSLCFLLFSGCFALREQAQQNECQIQKLNALKPDNRIESEGGFIETWNPNNKPFQ
CAGVALSRCTLNRNALRRPSYTNGPQEIYIQQGNGIFGMIFPGCPSTYQEPQESQQRGRS
QRPQDRHQKVHRFREGDLIAVPTGVAWWMYNNEDTPVVAVSIIDTNSLENQLDQMPR
RFYLAGNQEQEFLKYQQQQQGGSQSQKGKQQEEENEGSNILSGFAPEFLKEAFGVNMQI
VRNLQGENEEEDSGAIVTVKGGLRVTAPAMRKPQQEEDDDDEEEQPQCVETDKGCQRQ
SKRSRNGIDETICTMRLRQNIGQNSSPDIYNPQAGSITTATSLDFPALWLLKLSAQYGSLR
KNAMFVPHYTLNANSIIYALNGRALVQVVNCNGERVFDGELQEGGVLIVPQNFAVAAK
SQSDNFEYVSFKTNDRPSIGNLAGANSLLNALPEEVIQHTFNLKSQQARQVKNNNPFSFL
VPPQESQRRAVA
SEQ ID NO: 18
Gly m 6 Glycinin G3 121278 [MW = 54241.73 Da]
MAKLVLSLCFLLFSGCCFAFSFREQPQQNECQIQRLNALKPDNRIESEGGFIETWNPNNK
PFQCAGVALSRCTLNRNALRRPSYTNAPQEIYIQQGSGIFGMIFPGCPSTFEEPQQKGQSS
RPQDRHQKIYHFREGDLIAVPTGFAYWMYNNEDTPVVAVSLIDTNSFQNQLDQMPRRF
YLAGNQEQEFLQYQPQKQQGGTQSQKGKRQQEEENEGGSILSGFAPEFLEHAFVVDRQI
VRKLQGENEEEEKGAIVTVKGGLSVISPPTEEQQQRPEEEEKPDCDEKDKHCQSQSRNGI
DETICTMRLRHNIGQTSSPDIFNPQAGSITTATSLDFPALSWLKLSAQFGSLRKNAMFVPH
YNLNANSIIYALNGRALVQVVNCNGERVFDGELQEGQVLIVPQNFAVAARSQSDNFEY
VSFKTNDRFSIGNLAGANSLLNALPEEVIQQTFNLRRQQARQVKNNNPFSFLVPPKESQR
RVVA
SEQ ID NO: 19
Gly m 6 Glycinin G4 121279 [MW = 63587.16 Da]
MGKPFTLSLSSLCLLLLSSACFAISSSKLNECQLNNLNALEPDHRVESEGGLIQTWNSQHP
ELKCAGVTVSKLTLNRNGLHSPSYSPYPRMIIIAQGKGALGVAIPGCPETFEEPQEQSNRR
GSRSQKQQLQDSHQKIRHFNEGDVLVIPPSVPYWTYNTGDEPVVAISLLDTSNFNNQLD
QTPRVFYLAGNPDIEYPETMQQQQQQKSHGGRKQGQHQQEEEEEGGSVLSGFSKHFLA
QSFNTNEDIAEKLESPDDERKQIVTVEGGLSVISPKWQEQQDEDEDEDEDDEDEQIPSHP
PRRPSHGKREQDEDEDEDEDKPRPSRPSQGKRNKTGQDEDEDEDEDQPRKSREWRSKK
TQPRRPRQEEPRERGCETRNGVEENICTLKLHENIARPSRADFYNPKAGRISTLNSLTLPA
LRQFQLSAQYVVLYKNGIYSPHWNLNANSVIYVTRGQGKVRVVNCQGNAVFDGELRR
GQLLVVPQNFVVAEQAGEQGFEYIVFKTHHNAVTSYLKDVFRAIPSEVLAHSYNLRQSQ
VSELKYEGNWGPLVNPESQQGSPRVKVA
SEQ ID NO: 20
Gly m 6 Glycinin precursor 75221455 [MW = 63876.47 Da]
MGKPFTLSLSSLCLLLLSSACFAISSSKLNECQLNNLNALEPDHRVEFEGGLIQTWNSQHP
ELKCAGVTVSKLTLNRNGLHLPSYSPYPRMIIIAQGKGALQCKPGCPETFEEPQEQSNRR
GSRSQKQQLQDSHQKIRHFNEGDVLVIPPGVPYWTYNTGDEPVVAISLLDTSNFNNQLD
QTPRVFYLAGNPDIEYPETMQQQQQQKSHGGRKQGQHQQEEEEEGGSVLSGFSKHFLA
QSFNTNEDIAEKLQSPDDERKQIVTVEGGLSVISPKWQEQQDEDEDEDEDDEDEQIPSHP
PRRPSHGKREQDEDEDEDEDKPRPSRPSQGKREQDQDQDEDEDEDEDQPRKSREWRSK
KTQPRRPRQEEPRERGCETRNGVEENICTLKLHENIARPSRADFYNPKAGRISTLNSLTLP
ALRQFQLSAQYVVLYKNGIYSPHWNLNANSVIYVTRGQGKVRVVNCQGNAVFDGELR
RGQLLVVPQNFVVAEQAGEQGFEYIVFKTHHNAVTSYLKDVFRAIPSEVLAHSYNLRQS
QVSELKYEGNWGPLVNPESQQGSPRVKVA
SEQ ID NO: 21
Gly m Bd 28 K 12697782 [MW = 52944.36 Da]
MGNKTTLLLLLFVLCHGVATTTMAFHDDEGGDKKSPKSLFLMSNSTRVFKTDAGEMR
VLKSHGGRIFYRHMHIGFISMEPKSLFVPQYLDSNLIIFIRRGEAKLGFIYDDELAERRLKT
GDLYMIPSGSAFYLVNIGEGQRLHVICSIDPSTSLGLETFQSFYIGGGANSHSVLSGFEPAI
LETAFNESRTVVEEIFSKELDGPIMFVDDSHAPSLWTKFLQLKKDDKEQQLKKMMQDQ
EEDEEEKQTSRSWRKLLETVFGKVNEKIENKDTAGSPASYNLYDDKKADFKNAYGWSK
ALHGGEYPPLSEPDIGVLLVKLSAGSMLAPHVNPISDEYTIVLSGYGELHIGYPNGSRAM
KTKIKQGDVFVVPRYFPFCQVASRDGPLEFFGFSTSARKNKPQFLAGAASLLRTLMGPEL
SAAFGVSEDTLRRAVDAQHEAVILPSAWAAPPENAGKLKMEEEPNAIRSFANDVVMDV
F
SEQ ID NO: 22
Gly m Bd 30K 84371705 [MW = 42757.81 Da]
MGFLVLLLFSLLGLSSSSSISTHRSILDLDLTKFTTQKQVSSLFQLWKSEHGRVYHNHEEE
AKRLEIFKNNLNYIRDMNANRKSPHSHRLGLNKFADITPQEFSKKYLQAPKDVSQQIKM
ANKKMKKEQYSCDHPPASWDWRKKGVITQVKYQGGCGSGWAFSATGAIEAAHAIATG
DLVSLSEQELVDCVEESEGCYNGWHYQSFEWVLEHGGIATDDDYPYRAKEGRCKANKI
QDKVTIDGYETLIMSDESTESETEQAFLSAILEQPISVSIDAKDFHLYTGGIYDGENCTSPY
GINHFVLLVGYGSADGVDYWIAKNSWGEDWGEDGYIWIQRNTGNLLGVCGMNYFASY
PTKEESETLVSARVKGHRRVDHSPL
SEQ ID NO: 23
KTI 1 125722 [MW = 22545.94 Da]
MKSTIFFALFLVCAFTISYLPSATAQFVLDTDDDPLQNGGTYYMLPVMRGKGGGIEVDS
TGKEICPLTVVQSPNELDKGIGLVFTSPLHALFIAERYPLSIKFGSFAVITLCAGMPTEWAI
VEREGLQAVKLAARDTVDGWFNIERVSREYNDYKLVFCPQQAEDNKCEDIGIQIDDDGI
RRLVLSKNKPLVVQFQKFRSSTA
SEQ ID NO: 24
KTI 3 125020 [MW = 24005.29 Da]
MKSTIFFLFLFCAFTTSYLPSAIADFVLDNEGNPLENGGTYYILSDITAFGGIRAAPTGNER
CPLTVVQSRNELDKGIGTIISSPYRIRFIAEGHPLSLKFDSFAVIMLCVGIPTEWSVVEDLP
EGPAVKIGENKDAMDGWFRLERVSDDEFNNYKLVFCPQQAEDDKCGDIGISIDHDDGT
RRLVVSKNKPLVVQFQKLDKESLAKKNHGLSRSE
SEQ ID NO: 25
Gly m 8 (2S albumin) NP_001238443 [MW = 18459.97 Da]
MTKFTILLISLLFCIAHTCSASKWQHQQDSCRKQLQGVNLTPCEKHIMEKIQGRGDDDD
DDDDDNHILRTMRGRINYIRRNEGKDEDEEEEGHMQKCCTEMSELRSPKCQCKALQKI
MENQSEELEEKQKKKMEKELINLATMCRFGPMIQCDLSSDD
SEQ ID NO: 26
Lectin ADC94422 [MW = 30186.22 Da]
MATSNFSIVLSLSLAFFLVLLTKANSTNTVSFTVSKFSPRQQNLIFQGDAAISPSGVLRLT
KVDSIDVPTTGSLGRALYATPIQIWDSETGKVASWATSFKFKVFSPNKTADGLAFFLAPV
GSKPQSKGGFLGLFNSDSKNKSVQTVAVEFDTYYNAKWDPANRHIGIDVNSIKSVKTAS
WGLANGQIAQILITYDADTSLLVASLIHPSRKTSYILSETVSLKSNLPEWVNIGFSATTGL
NKGFVETHDVFSWSFASKLSDGSTSDTLDLPSFLLNEAI
SEQ ID NO: 27
Lipoxygenase CAA39604 [MW = 96817.14 Da]
MFGIFDKGQKIKGTVVLMPKNVLDFNAITSIGKGGVIDTATGILGQGVSLVGGVIDTATS
FLGRNISMQLISATQTDGSGNGKVGKEVYLEKHLPTLPTLGARQDAFSIFFEWDASFGIP
GAFYIKNFMTDEFFLVSVKLEDIPNHGTIEFVCNSWVYNFRSYKKNRIFFVNDTYLPSAT
PAPLLKYRKEELEVLRGDGTGKRKDFDRIYDYDVYNDLGNPDGGDPRPILGGSSIYPYP
RRVRTGRERTRTDPNSEKPGEVYVPRDENFGHLKSSDFLTYGIKSLSHDVIPLFKSAIFQL
RVTSSEFESFEDVRSLYEGGIKLPTDILSQISPLPALKEIFRTDGENVLQFPPPHVAKVSKS
GWMTDEEFAREVIAGVNPNVIRRLQEFPPKSTLDPTLYGDQTSTITKEQLEINMGGVTVE
EALSTQRLFILDYQDAFIPYLTRINSLPTAKAYATRTILFLKDDGTLKPLAIELSKPHPDGD
NLGPESIVVLPATEGVDSTIWLLAKAHVIVNDSGYHQLVSHWLNTHAVMEPFAIATNRH
LSVLHPIYKLLYPHYRDTININGLARQSLINADGIIEKSFLPGKYSIEMSSSVYKNWVFTD
QALPADLVKRGLAIEDPSAPHGLRLVIEDYPYAVDGLEIWDAIKTWVHEYVSLYYPTDA
AVQQDTELQAWWKEAVEKGHGDLKEKPWWPKMQTTEDLIQSCSIIVWTASALHAAVN
FGQYPYGGLILNRPTLARRFIPAEGTPEYDEMVKNPQKAYLRTITPKFETLIDLSVIEILSR
HASDEIYLGERETPNWTTDKKALEAFKRFGSKLTGIEGKINARNSDPSLRNRTGPVQLPY
TLLHRSSEEGLTFKGIPNSISI
SEQ ID NO: 28
Gly m Bd 28 K consensus sequence
VLCHGVATTTMAFHDDEGGDKKSPKSLFLMSNSTRVFKTDAGEMRVLKSHGGRIFYRH
MHIGFISMEPKSLFVPQYLDSNLIIFIRRGEAKLGFIYDDELAERRLKTGDLYMIPSGSAFY
LVNIGEGQRLHVICSIDPSTSLGLETFQSFYIGGGANSHSVLSGFEPAILETAFNESRTVVE
EIFSKELDGPIMFVDDSHAPSLWTKFLQLKKDDKEQQLKKMMQDQEEDEEEKQTSRSW
RKLLETVFGKVNEKIENKDTAGSPASYNLYDDKKADFKNAYGWSKALHGGEYPPLSEP
DIGVLLVKLSAGSMLAPHVNPISDEYTIVLSGYGELHIGYPNGSKAMKTKIKQGDVFVVP
RYFPFCQVASRDGPLEFFGFSTSARKNKPQFLAGAASLLRTLMGPELSAAFGVSEDTLRR
AVDAQHEAVILPSAWAAPPENAGKLKMEEEP
SEQ ID NO: 29
Gly m Bd 28 K Eric BAB21619 473 aa
KTTLLLLLFVLCHGVATTTMAFHDDEGGDKKSPKSLFLMSNSTRVFKTDAGEMRVLKS
HGGRIFYRHMHIGFISMEPKSLFVPQYLDSNLIIFIRRGEAKLGFIYDDELAERRLKTGDL
YMIPSGSAFYLVNIGEGQRLHVICSIDPSTSLGLETFQSFYIGGGANSHSVLSGFEPAILET
AFNESRTVVEEIFSKELDGPIMFVDDSHAPSLWTKFLQLKKDDKEQQLKKMMQDQEED
EEEKQTSRSWRKLLETVFGKVNEKIENKDTAGSPASYNLYDDKKADFKNAYGWSKAL
HGGEYPPLSEPDIGVLLVKLSAGSMLAPHVNPISDEYTIVLSGYGELHIGYPNGSRAMKT
KIKQGDVFVVPRYFPFCQVASRDGPLEFFGFSTSARKNKPQFLAGAASLLRTLMGPELSA
AFGVSEDTLRRAVDAQHEAVILPSAWAAPPENAGKLKMEEEPNAIRSFANDVVMDVF
SEQ ID NO: 30
Gly m Bd 28 K Ping ACD36978.1 455 aa
VLCHGVATTTMAFHDDEGGDKKSPKSLFLMSNSTRVFKTDAGEMRVLKSHGGRIFYRH
MHIGFISMEPKSLFVPQYLDSNLIIFIRRGEAKLGFIYDDELAERRLKTGDLYMIPSGSAFY
LVNIGEGQRLHVICSIDPSTSLGLETFQSFYIGGGANSHSVLSGFEPAILETAFNESRTVVE
EIFSKELDGPIMFVDDSHAPSLWTKFLQLKKDDKEQQLKKMMQDQEEDEEEKQTSRSW
RKLLETVFGKVNEKIENKDTAGSPASYNLYDDKKADFKNAYGWSKALHGGEYPPLSEP
DIGVLLVKLSAGSMLAPHVNPISDEYTIVLSGYGELHIGPNGSKAMKTKIKQGDVFVVPR
YFPFCQVASRDGPLEFFGFSTSARKNKPQFLAGAASLLRTLMGPELSAAFGVSEDTLRRA
VDAQHEAVILPSAWAAPRKMQEAEMEESQMLLKLCQ
SEQ ID NO: 31
Gly m Bd 28 K ACD36975.1 373 aa
LDSNLIIFIRRGEAKLGFIYDDELAERRLKTGDLYMIPSGSAFYLVNIGEGQRLHVICSIDP
STSLGLETFQSFYIGGGANSHSVLSGFEPAILETAFNESRTVVEEIFSKELDGPIMFVDDSH
APSLWTKFLQLKKDDKEQQLKKMMQDQEEDEEEKQTSRSWRKLLETVFGKVNEKIEN
KDTAGSPASYNLYDDKKADFKNAYGWSKALHGGEYPPLSEPDIGVLLVKLSAGSMLAP
HVNPISDEYTIVLSGYGELHIGYPNGSKAMKTKIKQGDVFVVPRYFPFCQVASRDGPLEF
FGFSTSARKNKPQFLAGAASLLRTLMGPELSAAFGVSEDTLRRAVDAQHEAVILPSAWA
APPENAGKLKMEEEP
SEQ ID NO: 32
Gly m Bd 28 K Ping ACD36976.1 373 aa
LDSNLIIFIRRGEAKLGFIYDDELAERRLKTGDLYMIPSGSAFYLVNIGEGQRLHVICSIDP
STSLGLETFQSFNIGGGANSHSVLSGFEPAILETAFNESRTVVEETFSKELDGPIMFVDDS
HAPSLWTKFLQLKKDDKEQQLKKMMQDQEEDEEEKQTSRSWRKLLETVFGKVNEKIE
NKDTAGSPASYNLYDDKKADFKNAYGWSKALHGGEYPPLSEPDIGVLLVKLSAGSMLA
PHVNPISDEYTIVLSGYGELHIGYPNGSKAMKTKIKQGDVFVVPRYFPFCQVASRDGPLE
FFGFSTSARKNKPQFLAGAASLLRTLMGPELSAAFGVSEDTLRRAVDAQHAAVILPSAW
AAPPENAGKLKMEEEP
SEQ ID NO: 33
Gly m Bd 28 K Ping ACD36974.1 320 aa
LDSNLIIFIRRGEAKLGFIYDDELAERRLKTGDLYMIPSGSAFYLVNIGEGQRLHVICSIDP
STSLGLETFQSFYIGGGANSHSVLSGFEPAILETAFNESRTVVEEIFSKELDGPIMFVDDSH
VPSLWTKFLQLKKDDKEQQLKKMMQDQEEDEEEKQTSRSWRKLLETVFGKVNEKIEN
KDTAGSPASYNLYDDKKADFKNAYGWSKALHGGEYPPLSEPDIGVLLVKLSAGSMLAP
HVNPISDEYTIVLSGYGELHIGYPNGSKAMKTKIKQGDVFVVPRYFPFCQVASRDGPLEF
FGFSTSARKNKPQFLAGAASL
SEQ ID NO: 34
LGFIYDDELAER
SEQ ID NO: 35
TVVEEIFSK
SEQ ID NO: 36
MMQDQEEDEEEK
SEQ ID NO: 37
NAYGWSK
SEQ ID NO: 38
ALHGGEYPPLSEPDIGVLLVK
SEQ ID NO: 39
QGDVFVVPR
SEQ ID NO: 40
YFPFCQVASR
SEQ ID NO: 41
TLMGPELSAAFGVSEDTLR
SEQ ID NO: 42
SFANDVVMDVF
SEQ ID NO: 43
Gly m Bd 30 K Ping AAB09252.1 379 aa (also serves as
consensus sequence)
MGFLVLLLFSLLGLSSSSSISTHRSILDLDLTKFTTQKQVSSLFQLWKSEHGRVYHNHEEE
AKRLEIFKNNSNYIRDMNANRKSPHSHRLGLNKFADITPQEFSKKYLQAPKDVSQQIKM
ANKKMKKEQYSCDHPPASWDWRKKGVITQVKYQGGCGRGWAFSATGAIEAAHAIAT
GDLVSLSEQELVDCVEESEGSYNGWQYQSFEWVLEHGGIATDDDYPYRAKEGRCKAN
KIQDKVTIDGYETLIMSDESTESETEQAFLSAILEQPISVSIDAKDFHLYTGGIYDGENCTS
PYGINHFVLLVGYGSADGVDYWIAKNSWGEDWGEDGYIWIQRNTGNLLGVCGMNYFA
SYPTKEESETLVSARVKGHRRVDHSPL
SEQ ID NO: 44
Gly m Bd 30 K Ping P22895.1 379 aa
MGFLVLLLFSLLGLSSSSSISTHRSILDLDLTKFTTQKQVSSLFQLWKSEHGRVYHNHEEE
AKRLEIFKNNSNYIRDMNANRKSPHSHRLGLNKFADITPQEFSKKYLQAPKDVSQQIKM
ANKKMKKEQYSCDHPPASWDWRKKGVITQVKYQGGCGRGWAFSATGAIEAAHAIAT
GDLVSLSEQELVDCVEESEGSYNGWQYQSFEWVLEHGGIATDDDYPYRAKEGRCKAN
KIQDKVTIDGYETLIMSDESTESETEQAFLSAILEQPISVSIDAKDFHLYTGGIYDGENCTS
PYGINHFVLLVGYGSADGVDYWIAKNSWGFDWGEDGYIWIQRNTGNLLGVCGMNYFA
SYPTKEESETLVSARVKGHRRVDHSPL
SEQ ID NO: 45
SILDLDLTK
SEQ ID NO: 46
FTTQK
SEQ ID NO: 47
NNLNYIR
SEQ ID NO: 48
FADITPQEFSK
SEQ ID NO: 49
EQYSCDHPPASWDWR
SEQ ID NO: 50
VTIDGYETLIMSDESTESETEQAFLSAILEQPISVSIDAK
SEQ ID NO: 51
NTGNLLGVCGMNYFASYPTK
SEQ ID NO: 52
KTI 1 consensus sequence
MKSTIFFALFLVCAFTISYLPSATAQFVLDTDDDPLQNGGTYYMLPVMRGKGGGIEGAS
TGKEICPLTVVQSPNELDKGIGLVFSSPLHALFIAERYPLSIKFGSFAVISLCGGMPTKWAI
VEREGLQAVTLAARDTVDGWFNIERVSREYNDYKLVFCPQNAEDNKCEDIGIQIDNDGI
RRLVLSKNKPLVVQFQKFRSSTA
SEQ ID NO: 53
KTI 1 AAB23483.1 204 aa
MKSTIFFALFLVCAFTISYLPSATAQFVLDTDDDPLQNGGTYYMLPVMRGKSGGI
EGNSTGKEICPLTVVQSPNKHNKGIGLVFKSPLHALFIAERYPLSIKFDSFAVIPLC
GVMPTKWAIVEREGLQAVTLAARDTVDGWFNIERVSREYNDYYKLVFCPQEAE
DNKCEDIGIQIDNDGIRRLVLSKNKPLVVEFQKFRSSTA
SEQ ID NO: 54
KTI 1 CAA56343.1 208 aa
MKSTTSLALFLLCALTSSYQPSATADIVFDTEGNPIRNGGTYYVLPVIRGKGGGIEFAKTE
TETCPLTVVQSPFEGLQRGLPLIISSPFKILDITEGLILSLKFHLCTPLSLNSFSVDRYSQGSA
RRTPCQTHWLQKHNRCWFRIQRASSESNYYKLVFCTSNDDSSCGDIVAPIDREGNRPLIV
THDQNHPLLVQFQKVEAYESSTA
SEQ ID NO: 55
EICPLTVVQSPNELDK
SEQ ID NO: 56
EGLQAVK
SEQ ID NO: 57
LVFCPQQAEDNK
SEQ ID NO: 58
CEDIGIQIDDDGIR
SEQ ID NO: 59
LVLSK
SEQ ID NO: 60
NKPLVVQFQK
SEQ ID NO: 61
KTI 3 consensus sequence
MKSTIFFALFLFCAFTTSYLPSAIADFVLDNEGNPLENGGTYYILSDITAFGGIRAAPTGNE
RCPLTVVQSRNELDKGIGTIISSPYRIRFIAEGHPLSLKFDSFAVIMLCVGIPTEWSVVEDL
PEGPAVKIGENKDAMDGWFRLERVSDDEFNNYKLVFCPQQAEDDKCGDIGISIDHDDG
TRRLVVSKNKPLVVQFQKLDKESLAKKNHGLSRSE
SEQ ID NO: 62
KTI 3 CAA45777.1 217 aa
MKSTIFFALFLFCAFTTSYLPSAIADFVLDNEGNPLENGGTYYILSDITAFGGIRAAPTGNE
RCPLTVVQSRNELDKGIGTIISSPYRIRFIAEGHPLSLKFDSFAVIMLCVGIPTEWSVVEDL
PEGPAVKIGENKDAMDGWFRLERVSDDEFNNYKLVFCPQQAEDDKCGDIGISIDHDDG
TRRLVVSKNKPLVVQFQKLDKESLAKKNHGLSRSE
SEQ ID NO: 63
KTI 3 CAA45778.1 217 aa
MKSTIFFALFLFCAFTTSYLPSAIADFVLDNEGNPLDSGGTYYILSDITAFGGIRAAPTGNE
RCPLTVVQSRNELDKGIGTIISSPFRIRFIAEGNPLRLKFDSFAVIMLCVGIPTEWSVVEDL
PEGPAVKIGENKDAVDGWFRIERVSDDEFNNYKLVFCTQQAEDDKCGDIGISIDHDDGT
RRLVVSKNKPLVVQFQKVDKESLAKKNHGLSRSE
SEQ ID NO: 64
CPLTVVQSR
SEQ ID NO: 65
NELDK
SEQ ID NO: 66
IGENK
SEQ ID NO: 67
DAMDGWFR
SEQ ID NO: 68
LVFCPQQAEDDK
SEQ ID NO: 69
CGDIGISIDHDDGTR
SEQ ID NO: 70
LVVSK
SEQ ID NO: 71
NKPLVVQFQK
SEQ ID NO: 72
2S albumin Glyma13g36400.1 BLAST 174 aa
MPPPSLHFTSPINSKMTKFTILLISLLFCIAHTCSASKWQHQQDSCRKQLQGVNLTPCEKH
IMEKIQGRGDDDDDDDDDNHILRTMRGRINYIRRNEGKDEDEEEEGHMQKCCTEMSEL
RSPKCQCKALQKIMENQSEELEEKQKKKMEKELINLATMCRFGPMIQCDLSSDD
SEQ ID NO: 73
2S albumin NP 001234950 BLAST 158 aa
MTKFTILLISLLFCIAHTCSASEWQHQQDSCRKQLQGVNLTPCEKHIMEKIQGRGDDDDD
DDDDNHILRTMRGRINYIRRNEGKDEDEEEEGHMQKCRTEMSELRSPKCQCKALQKIM
ENQSEELEEKQKKKMEKELINLATMCRFGPMIQCDLSSDD
SEQ ID NO: 74
2S albumin Glyma12g34160.1 BLAST 156 aa
MTKLTILLIALLFIAHTCCASKWQQHQQESCREQLKGINLNPCEHIMEKIQAGRRGEDGS
DEDHILIRTMPGRINYIRKKEGKEEEEEGHMQKCCSEMSELKSPICQCKALQKIMDNQSE
QLEGKEKKQMERELMNLAIRCRLGPMIGCDLSSDD
SEQ ID NO: 75
WQHQQDSCR
SEQ ID NO: 76
QLQGVNLTPCEK
SEQ ID NO: 77
HIMEK
SEQ ID NO: 78
DEDEEEEGHMQK
SEQ ID NO: 79
CCTEMSELR
SEQ ID NO: 80
ELINLATMCR
SEQ ID NO: 81
FGPMIQCDLSSDD
SEQ ID NO: 82
Lectin consensus sequence
MATSNFSIVLSLSLAFFLVLLTKANSTNTVSFTFSKFSPRQPNLILQGDAAISSSGVLRLTK
VDSNGVPTSGSLGRALYAAPIQIWDSETGKVASWATSFKFNVFAPNKTADGLAFFLAPV
GSKPQSKGGFLGLFNSDSKDKSLQTVAVEFDTYSNKKWDPANRHIGIDVNSIKSVKTAS
WGLANGQVAQILITYDAATSLLVASLIHPSRKTSYILSETVSLKSNLPEWVSIGFSATTGL
NEGSVETHDVISWSFASKLSDGSTSDALDLPSFLLNEAI
SEQ ID NO: 83
Lectin XP_003535884 BLAST 280 aa
MATSNFSIVLSLSLALFLMLLTKANSTNTVSFTTSKFSPRQQNLILQGDAAISPSGVLRLT
KVDSYGVPTSRSLGRALYAAPIQIWDSETGKVASWATSFKFNVFSPDKTADGLAFFLAP
VGSKPQYKAGFLGLFNSDSKNMSLQTVAVEFDTYYNQKWDPASRHIGIDVNSIKSVKT
APWGFANGQVAQILITYNADTSLLVASLVHPSRKTSYILSETVSLKSNLPEWVNVGFSAT
TGANKGFAETHDVFSWSFASKLSDGSTSDTLDLASFLLNEAI
SEQ ID NO: 84
Lectin Glyma10g15480.1 BLAST
MATSNFSIVLSLSLALFLMLLTKANSTNTVSFTTSKFSPRQQNLILQGDAAISPSGVLRLT
KVDSYGVPTSRSLGRALYAAPIQIWDSETGKVASWATSFKFNVFSPDKTADGLAFFLAP
VGSKPQYKAGFLGLFNSDSKNMSLQTVAWDPASRHIGIDVNSIKSVKTAPWGFANGQV
AQILITYNADTSLLVASLVHPSRKTSYILSETVSLKSNLPEWVNVGFSATTGANKGFAET
HDVFSWSFASKLSDGSTSDTLDLASFLLNEAI
SEQ ID NO: 85
Lectin NP_001237210 BLAST 282 aa
MATSNFSIVLSVSLAFFLVLLTKAHSTDTVSFTFNKFNPVQPNIMLQKDASISSSGVLQLT
KVGSNGVPTSGSLGRALYAAPIQIWDSETGKVASWATSFKFNIFAPNKSNSADGLAFFL
APVGSQPQSDDGFLGLFNSPLKDKSLQTVAIEFDTFSNKKWDPANRHIGIDVNSIKSVKT
ASWGLSNGQVAEILVTYNAATSLLVASLIHPSKKTSYILSDTVNLKSNLPEWVSVGFSAT
TGLHEGSVETHDVISWSFASKLSDGSSNDALDLPSFVLNEAI
SEQ ID NO: 86
Lectin Glyma02g18090.1 BLAST
MKVLCIIFEFKQIKAMATSNFSIVLSVSLAFFLVLLTKAHSTDTVSFTFNKFNPVQPNIML
QKDASISSSGVLQLTKVGSNGVPTSGSLGRALYAAPIQIWDSETGKVASWATSFKFNIFA
PNKSNSADGLAFFLAPVGSQPQSDDGFLGLFNSPLKDKSLQTVAIEFDTFSNKKWDPAN
RHIGIDVNSIKSVKTASWGLSNGQVAEILVTYNAATSLLVASLIHPSKKTSYILSDTVNLK
SNLPEWVSVGFSATTGLHEGSVETHDVISWSFASKLSDGSSNDALDLPSFVLNEAI
SEQ ID NO: 87
Lectin ACU23599 BLAST 282 aa
MATSNFSIVLSVSLAFFLVLLTKAHPTDTVSFTFNKFNPVQPNIMLQKDASISSSGVLQLT
KVGSNGVPTSGSLGRALYAAPIQIWDSETGKVASWATSFKFNIFAPNKSNSADGLAFFL
APVGSQPQSDDGFLGLFNSPLKDKSLQTVAIEFDTFSNKKWDPANRHIGIDVDSIKSIKTA
SWGLSNGQVAEILVTYNAATSLLVASLIHPSKKTSYILSDTVNLKSNLPEWVSVGFSATT
GLHEGSVETHDVISWSFASKLSDGSSNDALDLPSFVLNEAI
SEQ ID NO: 88
Lectin CAH60173 BLAST 280 aa
MASSKFSTVISFSLALFLVLLTQANSTNIFSFNFQTFDSPNLIFQGDASVSSSGQLRLTKVK
GNGKPTAASLGRAFYSAPIQIWDSTTGNVASFATSFTFNILAPNKSNSADGLAFALVPVG
SQPKSNGGFLGLFDNATYDSSAQTVAVEFDTYSNPKWDPENRHIGIDVNSIESIRTASWG
LANGQNAEILITYDSSTKLLVASLVHPSRRTSYIVSERVDLKSVLPEWVSIGFSATTGLLE
GSIETHDVLSWSFASKLSDDTTSEGLNLANFVLNKIL
SEQ ID NO: 89
Lectin Glyma10g01620.2 BLAST 228 aa
MATSKFHTQKPLFVVLSVVVVLLTMTKVNSTKPFLSPGTSSCRTNRTLILQGDALVTSSR
KSLGRALYSTPIHIWDSEIGSVASFAASFNFTVYASDIANLADGLAFFLAPIDTQPQTRGG
YLGLYNNPSNSSWGLANDQVTNVLITYDASTNLLVASLVHPSQRSSYILSDVLDLKVAL
PEWVRIGFSATTGLNVASETHDVHSWSFSSNLPFGSSNTNPSDFAIFI
SEQ ID NO: 90
Lectin Glma02g01590.1 BLAST 285 aa
MATSKLKTQNVVVSLSLTLTLVLVLLTSKANSAETVSFSWNKFVPKQPNMILQGDAIVT
SSGKLQLNKVDENGTPKPSSLGRALYSTPIHIWDKETGSVASFAASFNFTFYAPDTKRLA
DGLAFFLAPIDTKPQTHAGYLGLFNENESGDQVVAVEFDTFRNSWDPPNPHIGINVNSIR
SIKTTSWDLANNKVAKVLITYDASTSLLVASLVYPSQRTSNILSDVVDLKTSLPEWVRIG
FSAATGLDIPGESHDVLSWSFASNLPHASSNIDPLDLTSFVLHEAI
SEQ ID NO: 91
VFSPNK
SEQ ID NO: 92
ANSTNTVSFTVSK
SEQ ID NO: 93
QQNLIFQGDAAISPSGVLR
SEQ ID NO: 94
TADGLAFFLAPVGSKPQSK
SEQ ID NO: 95
Lipoxygenase consensus sequence
MGGIFDKGQKIKGTVVLMPKNVLDFNAITSIGKGGVIDTATGILGAGVSLVGGVIDTATA
FLGRNISMQLISATQTDGSGNGKVGKEVYLEKHLPTLPTLGARQDAFSIFFEWDASFGIP
GAFYIKNFMTDEFFLVSVKLEDIPNHGTIEFVCNSWVYNFKSYKKNRIFFVNDTYLPSAT
PAPLLKYRKEELEVLRGDGTGKRKDFDRIYDYDVYNDLGNPDGGDPRPILGGSSIYPYP
RRVRTGRERTRTDPNSEKPGEVYVPRDENFGHLKSSDFLTYGIKSLSHDVIPLFKSAIFQL
RVTSSEFDSFEDVRSLYEGGIKLPTDILSQISPLPALKEIFRTDGENVLQFPPPHVAKVSKS
GWMTDEEFAREMIAGVNPNVIRRLQEFPPKSTLDPTLYGDQTSTITKEQLEINMGGVTVE
EALSTQRLFILDYQDAFIPYLTRINSLPTAKAYATRTILFLKDDGTLKPLAIELSKPHPDGD
NLGPESIVVLPATEGVDSTIWLLAKAHVIVNDSGYHQLVSHWLNTHAVMEPFAIATNRH
LSVLHPIYKLLYPHYRDTININGLARQSLINADGIIEKSFLPGKYSIEMSSSVYKNWVFTD
QALPADLVKRGLAIEDPSAPHGLRLVIEDYPYAVDGLEIWDAIKTWVHEYVSLYYPTDA
AVQQDTELQAWWKEAVEKGHGDLKDKPWWPKMQTTEDLIQSCSIIIWTASALHAAVN
FGQYPYGGLILNRPTLARRFIPEEGTPEYDEMVKNPQKAYLRTITPKFETLIDLSVIEILSR
HASDEIYLGERDTPNWTTDKKALEAFKKFGSKLTGIEGKINARNSDPSLRNRTGPV
QLPYTLLHRSSEEGLTFKGIPNSISI
SEQ ID NO: 96
Lipoxygenase 2IUK_A BLAST 864 aa
MFGIFDKGQKIKGTVVLMPKNVLDFNAITSIGKGGVIDTATGILGQGVSLVGGVIDTATS
FLGRNISMQLISATQTDGSGNGKVGKEVYLEKHLPTLPTLGARQDAFSIFFEWDASFGIP
GAFYIKNFMTDEFFLVSVKLEDIPNHGTIEFVCNSWVYNFRSYKKNRIFFVNDTYLPSAT
PAPLLKYRKEEFEVLRGDGTGKRKDFDRIYDYDVYNDLGNPDGGDPRPILGGCSIYPYP
LRVRTGRERTRTDPNSEKPGEVYVPRDENFGHLKSSDFLTYGIKSLSHDVIPLFKSAIFQL
RVTSSEFESFEDVRSLYEGGIKLPTDILSQISPLPALKEIFRTDGENVLQFPPPHVAKVSKS
GVMTDEEFAREVIAGVNPNVIRRLQEFPPKSTLDPTLYGDQTSTITKEQLEINMGGVTVE
EALSTQRLFILDYQDAFIPYLTRINSLPTAKAYATRTILFLKDDGTLKPLAIELSKPHPDGD
NLGPESIVVLPATEGVDSTIWLLAKAHVIVNDSGYHQLVSHWLNTHAVMEPFAIATNRH
LSVLHPIYKLLYPHYRDTININGLARQSLINADGIIEKSFLPGKYSIEMSSSVYKNWVFTH
QALPADLVKRGLAIEDPSAPHGLRLVIEDYPYAVDGLEIWDAIKTWVHEYVSLYYPTDA
AVQQDTELQAWWKEAVEKGHGDLKEKPWWPKKQTTEDLIQSCSIIVWTASALHAAVN
FGQYPYGGLILNRPTLARRFIPAEGTPEYDEMVKNPQKAYLRTITPKFETLIDLSVIEILSR
HASDEIYLGERETPNWTTDKKALEAFKRFGSKLTGIEGKINARNSDPSLRNRTGPVQLPY
TLLHRSSEEGLTFKGIPNSISI
SEQ ID NO: 97
Lipoxygenase NP_001238676 BLAST 864 aa
MFGIFDKGQKIKGTVVLMPKNVLDFNAITSIGKGGVIDTATGILGQGVSLVGGVIDTATS
FLGRNISMQLISATQTDGSGNGKVGKEVYLEKHLPTLPTLGARQDAFSIFFEWDASFGIP
GAFYIKNFMTDEFFLVSVKLEDIPNHGTIEFVCNSWVYNFRSYKKNRIFFVNDTYLPSAT
PAPLLKYRKEELEVLRGDGTGKRKDFDRIYDYDVYNDLGNPDGGDPRPILGGCSIYPYP
LRVRTGRERTRTDPNSEKPGEVYVPRDENFGHLKSSDFLTYGIKSLSHDVIPLFKSAIFQL
RVTSSEFESFEDVRSLYEGGIKLPTDILSQISPLPALKEIFRTDGENVLQFPPPHVAKVSKS
GWMTDEEFAREVIAGVNPNVIRRLQEFPPKSTLDPTLYGDQTSTITKEQLEINMGGVTVE
EALSTQRLFILDYQDAFIPYLTRINSLPTAKAYATRTILFLKDDGTLKPLAIELSKPHPDGD
NLGPESIVVLPATEGVDSTIWLLAKAHVIVNDSGYHQLVSHWLNTHAVMEPFAIATNRH
LSVLHPIYKLLYPHYRDTININGLARQSLINADGIIEKSFLPGKYSIEMSSSVYKNWVFTH
QALPADLVKRGLAIEDPSAPHGLRLVIEDYPYAVDGLEIWDAIKTWVHEYVSLYYPTDA
AVQQDTELQAWWKEAVEKGHGDLKEKPWWPKKQTTEDLIQSCSIIVWTASALHAAVN
FGQYPYGGLILNRPTLARRFIPAEGTPEYDEMVKNPQKAYLRTITPKFETLIDLSVIEILSR
HASDEIYLGERETPNWTTDKKALEAFKRFGSKLTGIEGKINARNSDPSLRNRTGPVQLPY
TLLHRSSEEGLTFKGIPNSISI
SEQ ID NO: 98
Lipoxygenase Glyma08g20220.1 BLAST 867 aa
MLGLFDKSHKIKGTVVLMPKSVLDINDLNSVKNGGVGGVVSGIFGAVADVTGQIVDTA
TAIFSRNVSFKLISATSTDAKGNGKVGNETFLEKHLPTLPTLGDRRDAYDIHFEWDANFG
IPGAFYIRNYTYDEFFLVSVTLEDIPNHGTIHFVCNSWVYNFKDYDKKDRIFFANKTYLP
SATPGPLVKYREEELKILRGDGTGERKEHERIYDYDVYNDLGNPDEDVKLARPVLGGSS
TYPYPRRVRTGRKATKKDPKSERPASELYMPRDEKFGHLKSSDFLTYGIKSLSQKLLPSL
ENVFDSDLTWNEFDSFEEVRDLYEGGIKVPTGVLSDISPIPIFKEIFRTDGESVLQFPPPHV
VQVTKSAWMTDDEFAREMIAGVNPNVIRLLKEFPPQSKLDPSLYGDQSSTITKEHLEIN
MDGVTVEEALNGQRLFILDYQDAFMPYLTRINALPSAKAYATRTILLLKDDGTLKPLAIE
LSKPHPSGDNLGAESKVVLPADQGVESTIWLLAKAHVIVNDSGYHQLMSHWLNTHAVT
EPFIIATNRRLSVLHPIYKLLYPHYRDTININGLARNALINAGGVIEESFLPGRYSIEMSSA
VYKNWVFTDQALPVDLIKRGMAVEDPSSPHGLRLAVEDYPYAVDGLEIWDAIKSWVQE
YVSLYYPTDLAIQQDTELQAWWKEVVEKGHGDLKDKPWWPKMQTRQELIQSCSTIIWI
ASALHAAVNFGQYPYGGFILNRPTLSRRWIPEPGTKEYDEMVESPQTAYLRTITPKRQTII
DLTVIEILSRHASDEIYLGERDNPNWTSDSKALEAFKKFGSKLAEIEGKITARNKDSNKK
NRYGPVQLPYTLLLPTSEEGLTFRGIPNSISI
SEQ ID NO: 99
Lipoxygenase P24095 BLAST 864 aa
MFGIFDKGQKIKGTVVLMPKNVLDFNAITSIGKGGVIDTATGILGQGVSLVGGVIDTATS
FLGRNISMQLISATQTDGSGNGKVGKEVYLEKHLPTLPTLGARQDAFSIFFEWDASFGIP
GAFYIKNFMTDEFFLVSVKLEDIPNHGTIEFVCNSWVYNFRSYKKNRIFFVNDTYLPSAT
PAPLLKYRKEELEVLRGDGTGKRKDFDRIYDYDVYNDLGNPDGGDPRPILGGSSIYPYP
RRVRTGRERTRTDPNSEKPGEVYVPRDENFGHLKSSDFLTYGIKSLSHDVIPLFKSAIFQL
RVTSSEFESFEDVRSLYEGGIKLPTDILSQISPLPALKEIFRTDGENVLQFPPPHVAKVSKS
GWMTDEEFAREVIAGVNPNVIRRLQEFPPKSTLDPTLYGDQTSTITKEQLEINMGGVTVE
EALSTQRLFILDYQDAFIPYLTRINSLPTAKAYATRTILFLKDDGTLKPLAIELSKPHPDGD
NLGPESIVVLPATEGVDSTIWLLAKAHVIVNDSGYHQLVSHWLNTHAVMEPFAIATNRH
LSVLHPIYKLLYPHYRDTININGLARQSLINADGIIEKSFLPGKYSIEMSSSVYKNWVFTD
QALPADLVKRGLAIEDPSAPHGLRLVIEDYPYAVDGLEIWDAIKTWVHEYVSLYYPTDA
AVQQDTELQAWWKEAVEKGHGDLKEKPWWPKMQTTEDLIQSCSIIVWTASALHAAVN
FGQYPYGGLILNRPTLARRFIPAEGTPEYDEMVKNPQKAYLRTITPKFETLIDLSVIEILSR
HASDEIYLGERETPNWTTDKKALEAFKRFGSKLTGIEGKINARNSDPSLRNRTGPVQLPY
TLLHRSSEEGLTFKGIPNSISI
SEQ ID NO: 100
Lipoxygenase Glyma07g00900.1 BLAST 866 aa
MTGGMFGRKGQKIKGTVVLMPKNVLDFNAITSVGKGSAKDTATDFLGKGLDALGHAV
DALTAFAGHSISLQLISATQTDGSGKGKVGNEAYLEKHLPTLPTLGARQEAFDINFEWD
ASFGIPGAFYIKNFMTDEFFLVSVKLEDIPNHGTINFVCNSWVYNFKSYKKNRIFFVNDT
YLPSATPAPLLKYRKEELEVLRGDGTGKRKDFDRIYDYDVYNDLGNPDGGDPRPILGGS
SIYPYPRRVRTGRERTRTDPNSEKPGEVYVPRDENFGHLKSSDFLTYGIKSLSHDVIPLFK
SAIFQLRVTSSEFESFEDVRSLYEGGIKLPTDILSQISPLPALKEIFRTDGENVLQFPPPHVA
KVSKSGWMTDEEFAREVIAGVNPNVIRRLQEFPPKSTLDPTLYGDQTSTITKEQLEINMG
GVTVEEALSTQRLFILDYQDAFIPYLTRINSLPTAKAYATRTILFLKDDGTLKPLAIELSKP
HPDGDNLGPESIVVLPATEGVDSTIWLLAKAHVIVNDSGYHQLVSHWLNTHAVMEPFAI
ATNRHLSVLHPIYKLLYPHYRDTININGLARQSLINADGIIEKSFLPGKYSIEMSSSVYKN
WVFTDQALPADLVKRGLAIEDPSAPHGLRLVIEDYPYAVDGLEIWDAIKTWVHEYVSLY
YPTDAAVQQDTELQAWWKEAVEKGHGDLKEKPWWPKMQTTEDLIQSCSIIVWTASAL
HAAVNFGQYPYGGLILNRPTLARRFIPAEGTPEYDEMVKNPQKAYLRTITPKFETLIDLS
VIEILSRHASDEIYLGERETPNWTTDKKALEAFKRFGSKLTGIEGKINARNSDPSLRNRTG
PVQLPYTLLHRSSEEGLTFKGIPNSISI
SEQ ID NO: 101
Lipoxygenase NP_001235189 BLAST 859 aa
MTGGMFGRKGQKIKGTVVLMPKNVLDFNAITSVGKGSAKDTATDFLGKGLDALGHAV
DALTAFAGHSISLQLISATQTDGSGKGKVGNEAYLEKHLPTLPTLGARQEAFDINFEWD
ASFGIPGAFYIKNFMTDEFFLVSVKLEDIPNHGTINFVCNSWVYNFKSYKKNRIFFVNDT
YLPSATPGPLVKYRQEELEVLRGDGTGKRRDFDRIYDYDIYNDLGNPDGGDPRPIIGGSS
NYPYPRRVRTGREKTRKDPNSEKPGEIYVPRDENFGHLKSSDFLTYGIKSLSQNVIPLFKS
IILNLRVTSSEFDSFDEVRGLFEGGIKLPTNILSQISPLPVLKEIFRTDGENTLQFPPPHVIRV
SKSGWMTDDEFAREMIAGVNPNVIRRLQEFPPKSTLDPATYGDQTSTITKQQLEINLGGV
TVEEAISAHRLFILDYHDAFFPYLTKINSLPIAKAYATRTILFLKDDGSLKPLAIELSKPAT
VSKVVLPATEGVESTIWLLAKAHVIVNDSGYHQLISHWLNTHAVMEPFAIATNRHLSVL
HPIYKLLYPHYKDTININGLARQSLINAGGIIEQTFLPGKYSIEMSSVVYKNWVFTDQALP
ADLVKRGLAVEDPSAPHGLRLVIEDYPYAVDGLEIWDAIKTWVHEYVSVYYPTNAAIQ
QDTELQAWWKEVVEKGHGDLKDKPWWPKLQTVEDLIQSCSIIIWTASALHAAVNFGQ
YPYGGYIVNRPTLARRFIPEEGTKEYDEMVKDPQKAYLRTITPKFETLIDISVIEILSRHAS
DEVYLGQRDNPNWTTDSKALEAFKKFGNKLAEIEGKITQRNNDPSLKSRHGPVQLPYTL
LHRSSEEGMSFKGIPNSISI
SEQ ID NO: 102
Lipoxygenase Glyma07g03910.1 BLAST 865 aa
MFGILGGNKGHKIKGTVVLMSKNVLDFNEIVSTTQGGLVGAATGIFGAATGIVGGVVD
GATAIFSRNIAIQLISATKTDGLGNGKVGKQTYLEKHLPSLPTLGDRQDAFSVYFEWDND
FGIPGAFYIKNFMQSEFFLVSVTLEDIPNHGTIHFVCNSWVYNAKSYKRDRIFFANKTYLP
NETPTPLVKYRKEELENLRGDGKGERKEYDRIYDYDVYNDLGNPDKSNDLARPVLGGS
SAYPYPRRGRTGRKPTTKDSKSESPSSSTYIPRDENFGHLKSSDFLTYGIKSIAQTVLPTFQ
SAFGLNAEFDRFDDVRGLFEGGIHLPTDALSKISPLPVLKEIFRTDGEQVLKFPPPHVIKVS
KSAWMTDEEFGREMLAGVNPCLIECLQVFPPKSKLDPTVYGDQTSTITKEHLEINLGGLS
VEQALSGNRLFILDHHDAFIAYLRKINDLPTAKSYATRTILFLKDDGTLKPLAIELSLPHP
RGDEFGAVSRVVLPADQGAESTIWLIAKAYVVVNDSCYHQLMSHWLNTHAVIEPFVIA
TNRHLSVLHPIYKLLLPHYRDTMNINGLARQSLINAGGIIEQSFLPGPFAVEMSSAVYKG
WVFTDQALPADLIKRGMAVEDPSSPYGLRLVIDDYPYAVDGLEIWSAIQTWVKDYVSL
YYATDDAVKKDSELQAWWKEAVEKGHGDLKDKPWWPKLNTLQDLIHICCIIIWTASAL
HAAVNFGQYPYGGFILNRPTLTRRLLPEPGTKEYGELTSNHQKAYLRTITGKTEALVDLT
VIEILSRHASDEVYLGQRDNPNWTDDTKAIQAFKKFGNKLKEIEDKISGRNKNSSLRNRN
GPAQMPYTVLLPTSGEGLTFRGIPNSISI
SEQ ID NO: 103
Lipoxygenase Glyma07g03920.2 BLAST 868 aa
MLIGSLLNRRPKIKGTVVLMTKNVFDVNDFMATTRGGPAAVAGGIFGAAQDIVGGIVD
GATAIFSRNIAIQLISATKSENALGHGKVGKLTYLEKHLPSLPNLGDRQDAFDVYFEWDE
SFGIPGAFYIKNYMQSEFFLVSFKLEDVPNHGTILFACNSWVYNAKLYKKDRIFFANKAY
LPNDTPTPLVKYRKEELENLRGDGRGERKELDRIYDYDVYNDLGNPDENDDLARPILGG
SSKHPYPRRGRTGRKPTKKDPRCERPTSDTYIPRDENFGHLKSSDFLTYAIKSLTQNVLP
QFNTAFGFNNEFDSFEDVRCLFDGGVYLPTDVLSKISPIPVLKEIFRTDGEQALKFPPPHVI
KVRESEWMTDEEFGREMLAGVNPGMIQRLQEFPPKSKLDPTEFGDQTSTITKEHLEINLG
GLTVEQALKGNKLFILDHHDAFIPFMNLINGLPTAKSYATRTILFLQDDGTLKPLAIELSL
PHPRGHEFGADSRVVLPPAAVNSAEGTIWLIAKAYVAVNDTGYHQLISHWLNTHATIEP
FVIATNRHLSVLHPIHKLLLPHYRDTMNINALARQSLINADGVIERSFLPGKYSLEMSSAV
YKSWVFTDQALPADLIKRGMAIEDPCAPHGLRLVIEDYPYAVDGLEIWDAIQTWVKNY
VSLYYPTDDAIKKDSELQAWWKEAVETGHGDLKDKPWWPKLNTPQDLVHICSIIIWIAS
ALHAAVNFGQYPYGGLILNRPTLTRRFLPEPGSKEYEELSTNYQKAYLRTITRKIEALVD
LSVIEILSRHASDEIYLGKRDSDDWTDDQKAIQAFEKFGTKLKEIEAKINSRNKDSSLRNR
NGPVQMPYTVLLPTSEEGLTFRGIPNSISI
SEQ ID NO: 104
Lipoxygenase Glyma08g20190.1 BLAST 860 aa
MYSGVKGLFNRSQKVKGTVVLMRKNVLDINSITSVRGLIGTGINIIGSTIDGLTSFLGRSV
CLQLISATKADGNGNGVVGKKTYLEGIITSIPTLGAGQSAFTIHFEWDADMGIPGAFLIKN
YMQVELFLVSLTLEDIPNQGSMHFVCNSWVYNSKVYEKDRIFFASETYVPSETPGPLVT
YREAELQALRGNGTGKRKEWDRVYDYDVYNDLGNPDSGENFARPVLGGSLTHPYPRR
GRTGRKPTKKDPNSEKPGEAYIPRDENFGHLKSSDFLTYGLKSLTRSFLPALKTVFDINFT
PNEFDSFEEVRALCEGGIKLPTDILSKISPLPVLKEIFRTDGESVLKFSVPDLIKVSKSAWM
TDEEFAREMIAGVNPCVIRRLQEFPPQSKLDPSVYGDQTSKMTIDHLEINLEGLTVDKAI
KDQRLFILDHHDTFMPFLRRIDESKSSKAYATRTILFLKDDGTLKPLAIELSLPHPGQQQL
GAYSKVILPANQGVESTIWLLAKAHVIVNDSCYHQLISHWLNTHAVIEPFVIATNRNLSIL
HPIYKLLFPHYRDTMNINALARQSLINADGFIEKTFLGGKYAVEISSSGYKNWVFLDQAL
PADLIKRGMAIEDSSCPNGLRLVIEDYPYAVDGLEIWDAIKTWVQEYVSLYYATNDAIK
KDHELQAWWKEVVEKGHGDLKDKPWWPKMQTLQELIQSCSTIIWIASALHAAVNFGQ
YPYGGFILNRPTLSRRWIPEEGTPEYDEMTKNPQKAYLRTITPKFQALVDLSVIEILSRHA
SDEVYLGQRDNPNWTSNPKAIEAFKKFGKKLAEIETKISERNHDPNLRNRTGPAQLPYT
VLLPTSETGLTFRGIPNSISI
SEQ ID NO: 105
SSDFLTYGIK
SEQ ID NO: 106
GTVVLMPK
SEQ ID NO: 107
NVLDFNAITSIGK
SEQ ID NO: 108
GGVIDTATGILGQGVSLVGGVIDTATSFLGR
SEQ ID NO: 109
IFFVNDTYLPSATPAPLLK
SEQ ID NO: 110
DENFGHLK
SEQ ID NO: 111
SLSHDVIPLFK
SEQ ID NO: 112
SLYEGGIK
SEQ ID NO: 113
TDGENVLQFPPPHVAK
SEQ ID NO: 114
INSLPTAK
SEQ ID NO: 115
TILFLK
SEQ ID NO: 116
HLSVLHPIYK
SEQ ID NO: 117
QSLINADGIIEK
SEQ ID NO: 118
FIPAEGTPEYDEMVK
SEQ ID NO: 119
ALEAFK
SEQ ID NO: 120
GIPNSISI

### DETAILED DESCRIPTION OF THE INVENTION

It is of significance to enable a sensitive multiplex assay that is capable of selectively detecting and measuring levels of proteins of interest. Currently, relevant technologies for protein expression detection rely heavily on traditional immunochemistry technologies which present a challenge to accommodate the volume of data required to generate per sample.

Soybean is a multi-billion dollar commodity due to its balanced composition of 2:2:1 protein, starch, and oil by weight. Many seeds, including soybeans, contain proteins that are allergens and anti-nutritional factors. As such, there are concerns regarding the potential of altering allergen levels in genetically-modified soybean varieties when compared to varieties developed through traditional breeding. The measurement of allergen levels in crops has been achieved almost exclusively by immunoassays, such as enzyme-linked immunosorbent assays (ELISA) or IgE-immunoblotting; however, these methods suffer from limited sensitivity and specificity and high variability.

There has been recent interest in developing LC-MS/MS based methods to quantify several plant-expressed proteins in a single analysis. Analysis using these “signature peptides” involves tracking protein expression levels by quantifying several highly specific digest fragments of the proteins of interest. This can be typically accomplished using liquid chromatography coupled with selected reaction monitoring (SRM) tandem mass spectrometry. Improved multiplexed LC-MS/MS methods and systems are provided herein to enable simultaneous quantitation(s) of several allergen proteins in transgenic and non-transgenic soybean. Methods and systems provided herein are validated for analytical figures of merit including accuracy, precision, linearity, limits of detection and quantitation; and for other considerations including sample throughput, transferability, and ease of use. The allergens can be quantified using a multiplexing format and samples can be harvested from the field, processed, and analyzed/quantitated for example within a day (twenty-four hours) window (from field to measured numerical value). In addition, sample preparations of the methods and systems provided can be fully scalable for high-throughput, thus enabling hundreds of samples to be analyzed in a single batch.

Representative soybean allergens include, for example, Gly m 1, Gly m 3, Gly m 4, Gly m 5 (beta-conglycinin), Gly m 6 (Glycinin) GI, Gly m 6 (Glycinin) G2, Gly m 6 (Glycinin) G3, Gly m 6 (Glycinin) G4, Gly m 6 (Glycinin) precursor, Gly m 6 (Glycinin) G4 precursor, Kunitz trypsin inhibitor 1, Kunitz trypsin inhibitor 3, Gly m Bd 28 K, Gly m Bd 30 K, Gly m 8 (2S albumin), Lectin, and lipoxygenase.

Representative wheat allergens include, for example, profilin (Tri a12), wheat lipid transfer protein 1 (Tri a14), agglutinin isolectin 1 (Tri a18), omega-5 gliadin—seed storage protein (Tri a19), gliadin (Tri a20; NCBI Accession Nos. M10092, M11073, M11074, M11075, M11076, K03074, and K03075), thioredoxin (Tri a25), high molecular weight glutenin (Tri a26), low molecular weight glutenin (Tri a36), and alpha purothionin (Tri a37).

Representative corn allergens include, for example, maize lipid transfer protein (LTP) (Zea m14) and thioredoxin (Zea m25).

Representative corn allergens include, for example, rice profilin A (Ory s12).

In some embodiments, the methods and systems provided use liquid chromatography coupled to tandem mass spectrometry (LC-MS/MS) to detect protein expression levels of sixteen different allergens from soybean. In some embodiments, the methods and systems enable analysis of each allergen by itself or combined with additional proteins for a multiplexing assay for qualitative and quantitative analysis in plant matrices.

In some embodiments, the mass spectrometry detection for quantitative studies may be accomplished using selected reaction monitoring, performed on a triple quadrupole mass spectrometer. Using this type of instrumentation, initial mass-selection of ion (peptide) of interest formed in the source, followed by, dissociation of this precursor ion in the collision region of the MS, then mass-selection, and counting, of a specific product (daughter) ion. In some embodiments, the mass spectrometry detection for quantitative studies may be accomplished using selected reaction monitoring (SRM). Using particular type of instrumentation, initial mass-selection of ion of interest formed in the source, followed by, dissociation of this precursor (protein) ion in the collision region of the mass spectrometer (MS), then mass-selection, and counting, of a specific product (peptide) ion. In some embodiment, counts per unit time may provide an integratable peak area from which amounts or concentration of analytes can be determined. In some embodiment, the use of high resolution accurate mass (HRAM) monitoring for quantitation, performed on a HRAM capable mass spectrometer, may include, but is not limited to, hybrid quadrupole-time-of-flight, quadrupole-orbitrap, ion trap-orbitrap, or quadrupole-ion-trap-orbitrap (tribrid) mass spectrometers. Using particular type of instrumentation, peptides are not subject to fragmentation conditions, but rather are measured as intact peptides using full scan or targeted scan modes (for example selective ion monitoring mode or SIM). Integratable peak area can be determined by generating an extracted ion chromatogram for each specific analyte and amounts or concentration of analytes can be calculated. The high resolution and accurate mass nature of the data enable highly specific and sensitive ion signals for the analyte (protein and/or peptide) of interest.

Unless otherwise stated, the following terms used in this application, including the specification and claims, have the definitions given below. It must be noted that, as used in the specification and the appended claims, the singular forms “a,” “an,” and “the” include plural referents unless the context clearly dictates otherwise.

As used herein, the term “bioconfinement” refers to restriction of the movement of genetically modified plants or their genetic material to designated areas. The term includes physical, physicochemical, biological confinement, as well as other forms of confinement that prevent the survival, spread or reproduction of a genetically modified plants in the natural environment or in artificial growth conditions.

As used herein, the term “complex protein sample” is used to distinguish a sample from a purified protein sample. A complex protein sample contains multiple proteins, and may additionally contain other contaminants.

As used herein, the general term “mass spectrometry” or “MS” refers to any suitable mass spectrometry method, device or configuration including, e.g., electrospray ionization (ESI), matrix-assisted laser desorption/ionization (MALDI) MS, MALDI-time of flight (TOF) MS, atmospheric pressure (AP) MALDI MS, vacuum MALDI MS, or combinations thereof. Mass spectrometry devices measure the molecular mass of a molecule (as a function of the molecule's mass-to-charge ratio) by measuring the molecule's flight path through a set of magnetic and electric fields. The mass-to-charge ratio is a physical quantity that is widely used in the electrodynamics of charged particles. The mass-to-charge ratio of a particular peptide can be calculated, a priori, by one of skill in the art. Two particles with different mass-to-charge ratio will not move in the same path in a vacuum when subjected to the same electric and magnetic fields.

Mass spectrometry instruments consist of three modules: an ion source, which splits the sample molecules into ions; a mass analyzer, which sorts the ions by their masses by applying electromagnetic fields; and a detector, which measures the value of an indicator quantity and thus provides data for calculating the abundances of each ion present. The technique has both qualitative and quantitative applications. These include identifying unknown compounds, determining the isotopic composition of elements in a molecule, determining the structure of a compound by observing its fragmentation, and quantifying the amount of a compound in a sample.

A detailed overview of mass spectrometry methodologies and devices can be found in the following references which are hereby incorporated by reference: Can and Annan (1997) Overview of peptide and protein analysis by mass spectrometry. In: Current Protocols in Molecular Biology, edited by Ausubel, et al. New York: Wiley, p. 10.21.1-10.21.27; Paterson and Aebersold (1995) Electrophoresis 16: 1791-1814; Patterson (1998) Protein identification and characterization by mass spectrometry. In: Current Protocols in Molecular Biology, edited by Ausubel, et al. New York: Wiley, p. 10.22.1-10.22.24; and Domon and Aebersold (2006) Science 312(5771):212-17.

As the term is used herein, proteins and/or peptides are “multiplexed” when two or more proteins and/or peptides of interest are present in the same sample.

As used herein, a “plant trait” may refer to any single feature or quantifiable measurement of a plant.

As used herein, the phrase “peptide” or peptides” may refer to short polymers formed from the linking, in a defined order, of α-amino acids. Peptides may also be generated by the digestion of polypeptides, for example proteins, with a protease.

As used herein, the phrase “protein” or proteins” may refer to organic compounds made of amino acids arranged in a linear chain and joined together by peptide bonds between the carboxyl and amino groups of adjacent amino acid residues. The sequence of amino acids in a protein is defined by the sequence of a gene, which is encoded in the genetic code. In general, the genetic code specifies 20 standard amino acids, however in certain organisms the genetic code can include selenocysteine—and in certain archaea-pyrrolysine. The residues in a protein are often observed to be chemically modified by post-translational modification, which can happen either before the protein is used in the cell, or as part of control mechanisms. Protein residues may also be modified by design, according to techniques familiar to those of skill in the art. As used herein, the term “protein” encompasses linear chains comprising naturally occurring amino acids, synthetic amino acids, modified amino acids, or combinations of any or all of the above.

As used herein, the term “single injection” refers to the initial step in the operation of a MS or LC-MS device. When a protein sample is introduced into the device in a single injection, the entire sample is introduced in a single step.

As used herein, the phrase “signature peptide” refers an identifier (short peptide) sequence of a specific protein. Any protein may contain an average of between 10 and 100 signature peptides. Typically signature peptides have at least one of the following criteria: easily detected by mass spectroscopy, predictably and stably eluted from a liquid chromatography (LC) column, enriched by reversed phase high performance liquid chromatography (RP-HPLC), good ionization, good fragmentation, or combinations thereof. A peptide that is readily quantified by mass spectrometry typically has at least one of the following criteria: readily synthesized, ability to be highly purified (>97%), soluble in ≤20% acetonitrile, low non-specific binding, oxidation resistant, post-synthesis modification resistant, and a hydrophobicity or hydrophobicity index ≥10 and ≤40. The hydrophobicity index can be calculated according to Krokhin, Molecular and Cellular Proteomics 3 (2004) 908, which is incorporated by reference. It's known that a peptide having a hydrophobicity index less than 10 or greater than 40 may not be reproducibly resolved or eluted by a RP-HPLC column.

As used herein, the term “stacked” refers to the presence of multiple heterologous polynucleotides incorporated in the genome of a plant.

Tandem mass spectrometry: In tandem mass spectrometry, a parent ion generated from a molecule of interest may be filtered in a mass spectrometry instrument, and the parent ion subsequently fragmented to yield one or more daughter ions that are then analyzed (detected and/or quantified) in a second mass spectrometry procedure. In some embodiments, the use of tandem mass spectrometry is excluded. In these embodiments, tandem mass spectrometry is not used in the methods and systems provided. Thus, neither parent ions nor daughter ions are generated in these embodiments.

As used herein, the term “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenic plants initially so altered as well as those created by sexual crosses or asexual propagation from the initial transgenic plant.

Any plants that provide useful plant parts may be treated in the practice of the present invention. Examples include plants that provide flowers, fruits, vegetables, and grains.

As used herein, the phrase “plant” includes dicotyledonous plants and monocotyledonous plants. Examples of dicotyledonous plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledonous plants include corn, rice, wheat, sugarcane, barley, rye, sorghum, orchids, bamboo, banana, cattails, lilies, oat, onion, millet, and triticale. Examples of fruit include banana, pineapple, oranges, grapes, grapefruit, watermelon, melon, apples, peaches, pears, kiwifruit, mango, nectarines, guava, persimmon, avocado, lemon, fig, and berries. Examples of flowers include baby's breath, carnation, dahlia, daffodil, geranium, gerbera, lily, orchid, peony, Queen Anne's lace, rose, snapdragon, or other cut-flowers or ornamental flowers, potted-flowers, and flower bulbs.

The specificity allowed in a mass spectrometry approach for identifying a single protein from a complex sample is unique in that only the sequence of the protein of interest is required in order to identify the protein of interest. Compared to other formats of multiplexing, mass spectrometry is unique in being able to exploit the full length of a protein's primary amino acid sequence to target unique identifier-type portions of a protein's primary amino acid sequence to virtually eliminate non-specific detection. In some embodiments of the present invention, a proteolytic fragment or set of proteolytic fragments that uniquely identifies a protein(s) of interest is used to detect the protein(s) of interest in a complex protein sample.

In some embodiments, disclosed methods enable the quantification or determination of ratios of multiple proteins in a complex protein sample by a single mass spectrometry analysis, as opposed to measuring each protein of interest individually multiple times and compiling the individual results into one sample result.

In some embodiments, the present disclosure also provides methods useful for the development and use of transgenic plant technology. Specifically, disclosed methods may be used to maintain the genotype of transgenic plants through successive generations. Also, some embodiments of the methods disclosed herein may be used to provide high-throughput analysis of non-transgenic plants that are at risk of being contaminated with transgenes from neighboring plants, for example, by cross-pollination. By these embodiments, bioconfinement of transgenes may be facilitated and/or accomplished. In other embodiments, methods disclosed herein may be used to screen the results of a plant transformation procedure in a high-throughput manner to identify transformants that exhibit desirable expression characteristics.

The mass-to-charge ratio may be determined using a quadrupole analyzer. For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as a “mass filter” and “mass detector” for the ions injected into the instrument.

Collision-induced dissociation (“CID”) is often used to generate the daughter ions for further detection. In CID, parent ions gain energy through collisions with an inert gas, such as argon, and subsequently fragmented by a process referred to as “unimolecular decomposition.” Sufficient energy must be deposited in the parent ion so that certain bonds within the ion can be broken due to increased energy.

The mass spectrometer typically provides the user with an ion scan; that is, the relative abundance of each m/z over a given range (for example 10 to 1200 amu). The results of an analyte assay, that is, a mass spectrum, can be related to the amount of the analyte in the original sample by numerous methods known in the art. For example, given that sampling and analysis parameters are carefully controlled, the relative abundance of a given ion can be compared to a table that converts that relative abundance to an absolute amount of the original molecule. Alternatively, molecular standards (e.g., internal standards and external standards) can be run with the samples and a standard curve constructed based on ions generated from those standards. Using such a standard curve, the relative abundance of a given ion can be converted into an absolute amount of the original molecule. Numerous other methods for relating the presence or amount of an ion to the presence or amount of the original molecule are well known to those of ordinary skill in the art.

The choice of ionization method can be determined based on the analyte to be measured, type of sample, the type of detector, the choice of positive versus negative mode, etc. Ions can be produced using a variety of methods including, but not limited to, electron ionization, chemical ionization, fast atom bombardment, field desorption, and matrix-assisted laser desorption ionization (MALDI), surface enhanced laser desorption ionization (SELDI), desorption electrospray ionization (DESI), photon ionization, electrospray ionization, and inductively coupled plasma. Electrospray ionization refers to methods in which a solution is passed along a short length of capillary tube, to the end of which is applied a high positive or negative electric potential. Solution reaching the end of the tube, is vaporized (nebulized) into a jet or spray of very small droplets of solution in solvent vapor. This mist of droplets flows through an evaporation chamber which is heated to prevent condensation and to evaporate solvent. As the droplets get smaller the electrical surface charge density increases until such time that the natural repulsion between like charges causes ions as well as neutral molecules to be released.

The effluent of an LC may be injected directly and automatically (i.e., “in-line”) into the electrospray device. In some embodiments, proteins contained in an LC effluent are first ionized by electrospray into a parent ion.

Various different mass analyzers can be used in liquid chromatography—mass spectrometry combination (LC-MS). Exemplary mass analyzers include, but not limited to, single quadrupole, triple quadrupole, ion trap, TOF (time of flight), and quadrupole-time of flight (Q-TOF).

The quadrupole mass analyzer may consist of 4 circular rods, set parallel to each other. In a quadrupole mass spectrometer (QMS), the quadrupole is the component of the instrument responsible for filtering sample ions, based on their mass-to-charge ratio (m/z). Ions are separated in a quadrupole based on the stability of their trajectories in the oscillating electric fields that are applied to the rods.

An ion trap is a combination of electric or magnetic fields that captures ions in a region of a vacuum system or tube. Ion traps can be used in mass spectrometry while the ion's quantum state is manipulated.

Time-of-flight mass spectrometry (TOFMS) is a method of mass spectrometry in which an ion's mass-to-charge ratio is determined via a time measurement. Ions are accelerated by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle (heavier particles reach lower speeds). From this time and the known experimental parameters one can find the mass-to-charge ratio of the ion.

In some embodiments, the particular instrument used by the methods and/or systems provided may comprise a high fragmentation mode and a low fragmentation mode (or alternatively a non-fragmentation mode). Such different modes may include alternating scan high and low energy acquisition methodology to generate high resolution mass data. In some embodiments, the high resolution mass data may comprise a product data set (for example data derived from product ion (fragmented ions) under the high fragmentation mode) and a precursor data set (for example data derived from precursor ions (unfragmented ions) under the low fragmentation or non-fragmentation mode).

In some embodiments, the methods and/or systems provided use a mass spectrometer comprising a filtering device that may be used in the selection step, a fragmentation device that may be used in the fragmentation step, and/or one or more mass analyzers that may be used in the acquisition and/or mass spectrum creation step or steps.

The filtering device and/or mass analyzer may comprise a quadrupole. The selection step and/or acquisition step and/or mass spectrum creation step or steps may involve the use of a resolving quadrupole. Additionally or alternatively, the filtering device may comprise a two-dimensional or three-dimensional ion trap or time-of-flight (ToF) mass analyzer. The mass analyzer or mass analyzers may comprise or further comprise one or more of a time-of-flight mass analyzer and/or an ion cyclotron resonance mass analyzer and/or an orbitrap mass analyzer and/or a two-dimensional or three-dimensional ion trap.

Filtering by means of selection based upon mass-to-charge ratio (m/z) can be achieved by using a mass analyzer which can select ions based upon m/z, for example a quadrupole; or to transmit a wide m/z range, separate ions according to their m/z, and then select the ions of interest by means of their m/z value. An example of the latter would be a time-of-flight mass analyzer combined with a timed ion selector(s). The methods and/or systems provided may comprise isolating and/or separating the one or more proteins of interest, for example from two or more of a plurality of proteins, using a chromatographic technique for example liquid chromatography (LC). The method may further comprise measuring an elution time for the protein of interest and/or comparing the measured elution time with an expected elution time.

Additionally or alternatively, the proteins of interest may be separated using an ion mobility technique, which may be carried out using an ion mobility cell. Additionally, the proteins of interest may be selected by order or time of ion mobility drift. The method may further comprise measuring a drift time for the proteins of interest and/or comparing the measured drift time with an expected drift time.

In some embodiments, the methods and/or systems provided are label-free, where quantitation can be achieved by comparison of the peak intensity, or area under the mass spectral peak for the precursor or product m/z values of interest between injections and across samples. In some embodiments, internal standard normalization may be used to account for any known associated analytical error. Another label-free method of quantification, spectral counting, involves summing the number of fragment ion spectra, or scans, that are acquired for each given peptide, in a non-redundant or redundant fashion. The associated peptide mass spectra for each protein are then summed, providing a measure of the number of scan's per protein with this being proportional to its abundance. Comparison can then be made between samples/injections.

In some embodiments, the ion source is selected from the group consisting of: (1) an electrospray ionization (“ESI”) ion source; (2) an atmospheric pressure photo ionization (“APPI”) ion source; (3) an atmospheric pressure chemical ionization (“APCI”) ion source; (4) a matrix assisted laser desorption ionization (“MALDI”) ion source; (5) a laser desorption ionization (“LDI”) ion source; (6) an atmospheric pressure ionization (“API”) ion source; (7) a desorption ionization on silicon (“DIOS”) ion source; (8) an electron impact (“E1”) ion source; (9) a chemical ionization (“CI”) ion source; (10) a field ionization (“F1”) ion source; (11) a field desorption (“FD”) ion source; (12) an inductively coupled plasma (“ICP”) ion source; (13) a fast atom bombardment (“FAB”) ion source; (14) a liquid secondary ion mass spectrometry (“LSIMS”) ion source; (15) a desorption electrospray ionization (“DESI”) ion source; (16) a nickel-63 radioactive ion source; (17) an atmospheric pressure matrix assisted laser desorption ionization ion source; and (18) a thermospray ion source.

In some embodiments, the methods and/or systems provided comprise an apparatus and/or control system configured to execute a computer program element comprising computer readable program code means for causing a processor to execute a procedure to implement the methods.

In some embodiments, the methods and/or systems provided use an alternating low and elevated energy scan function in combination with liquid chromatography separation of a plant extract. A list of information for proteins of interest can be provided including, but is not limited to, m/z of precursor ion, m/z of product ions, retention time, ion mobility drift time and rate of change of mobility. During the course of the LC separation and as the target ions elute into the mass spectrometer (and as either low energy precursor ions, or elevated energy product ions are detected, or the retention time window is activated) the mass analyzer of the methods and/or systems provided may select a narrow m/z range (of a variable and changeable width) to pass ions through to the gas cell. Accordingly, the signal to noise ratio can be enhanced significantly for quantification of proteins of interest.

In some embodiments, at a chromatographic retention time when a targeted protein of interest is about to elute into the mass spectrometer ion source, the mass analyzer of the methods and/or systems provided can select a narrow m/z range (of a variable and changeable width) according to the targeted precursor ion. These selected ions are then transferred to an instrument stage capable of dissociating the ions by means of alternate and repeated switches between a high fragmentation mode where the sample precursor ions are substantially fragmented into product ions and a low fragmentation mode (or non-fragmentation mode) where the sample precursor ions are not substantially fragmented. Typically high resolution, accurate mass spectra are acquired in both modes and at the end of the experiment associated precursor and product ions are recognized by the closeness in fit of their chromatographic elution times and optionally other physicochemical properties. The signal intensity of either the precursor ion or the product ion associated with targeted proteins of interest can be used to determine the quantity of the proteins in the plant extract.

Those skilled in the art would understand certain variation can exist based on the disclosure provided. Thus, the following examples are given for the purpose of illustrating the invention and shall not be construed as being a limitation on the scope of the invention or claims.

### EXAMPLES

### Example 1

The methods and systems provided are used for determination of endogenous soybean allergen proteins in soybean seed including Gly m 1, Gly m 3, Gly m 4, Gly m 5 (beta-conglycinin), Gly m 6, Kunitz trypsin inhibitor 1, Kunitz trypsin inhibitor 3, Gly m Bd 28 K, Gly m Bd 30 K, and Gly m 8 (2S albumin).

TABLE 1
Preparation of signature peptide calibration standards

| Initial | | Volume of | Volume | Final |
|---|---|---|---|---|
| concentration | | Dilution | of Std. | concentration |
| (ng/mL) | Standard | Cocktail (μL) | (μL) | (ng/mL) |
| 5880.00 | Std 12 | — | — | 500.00 |
| 500.00 | Std 11 | 200 | 200 | 250.00 |
| 250.00 | Std 10 | 200 | 200 | 125.00 |
| 125.00 | Std 9 | 200 | 200 | 62.50 |
| 62.50 | Std 8 | 200 | 200 | 31.25 |
| 31.25 | Std 7 | 200 | 200 | 15.63 |
| 15.63 | Std 6 | 200 | 200 | 7.81 |
| 7.81 | Std 5 | 200 | 200 | 3.91 |
| 3.91 | Std 4 | 200 | 200 | 1.95 |
| 1.95 | Std 3 | 200 | 200 | 0.98 |
| 0.98 | Std 2 | 200 | 200 | 0.49 |
| 0.49 | Std 1 | 2000 | 2000 | 0.24 |

A 100±0.5 mg ground soybean seed sample is defatted twice with hexanes and dried before extracting with extraction buffer containing 5 M urea, 2 M thiourea, 50 mM Tris pH 8.0 and 65 mM DTT. The sample is sonicated in a water bath for thirty minutes, vortexed for one minute, sonicated for another thirty minutes and centrifuged at >3,000 rpm for ten minutes at 4° C.

The aqueous supernatant is collected and diluted to bring the endogenous soybean allergen protein concentration into the calibration standard range with extraction buffer. The diluted extract is denatured at 95° C. for twenty minutes with the additional 1 M Tris pH 8.0, 0.5 M DTT and deionized water followed by refrigeration at 4° C. for ten minutes. The denatured extract is incubated overnight (˜15 hours) at 37° C. with 0.5 mg/mL trypsin enzyme. The digestion reaction is quenched with formic acid water (50/50 v/v) and centrifuge at >3,000 rpm for tern minutes at 4° C. An aliquot of digested extract is transferred to an autosampler vial and analyzed along with calibration standard by liquid chromatography with positive-ion electrospray (ESI) tandem mass spectrometry (LC-MS/MS). Calibration standards of signature peptides are prepared as listed in Table 1.

The limits of detection (LOD) and limits of quantitation (LOQ) for endogenous soybean allergens in this example are set forth in Table 2, where LOD and LOQ represent protein concentration (ng/mg).

TABLE 2
Limits of detection (LOD) and limits of quantita-
tion (LOQ) for endogenous soybean allergens in
Example 1 (LOD and LOQ represent protein
concentration)

| | | LOD | LOQ |
|---|---|---|---|
| Allergen | Signature peptide | (ng/mg) | (ng/mg) |
| Gly m 1 | SYPSNATCPR | 0.23 | 0.46 |
| | (SEQ ID NO: 1) | | |
| Gly m 3 | YMVIQGEPGAVIR | 0.20 | 0.39 |
| | (SEQ ID NO: 2) | | |
| Gly m 5 | NILEASYDTK | 1.22 | 2.44 |
| | (SEQ ID NO: 3) | | |
| Glycinin G2 | VTAPAMR | 1.46 | 2.92 |
| | (SEQ ID NO: 4) | | |
| Glycinin G3 | NNNPFSFLVPPK | 1.58 | 3.16 |
| | (SEQ ID NO: 5) | | |
| Glycinin | ADFYNPK | — | — |
| precursor | (SEQ ID NO: 6) | | |
| Kunitz trypsin | GGGIEVDSTGK | — | — |
| inhibitor 1 | (SEQ ID NO: 7) | | |
| Kunitz trypsin | GIGTLLSSPYR | — | — |
| inhibitor 3 | (SEQ ID NO: 8) | | |
| Gly m Bd 28 K | NKPQFLAGAASLLR | 5.70 | 11.40 |
| | (SEQ ID NO: 9) | | |
| Gly m Bd 30 K | GVITQVK | 1.15 | 2.30 |
| | (SEQ ID NO: 10) | | |
| Gly m 8 | IMENQSEELEEK | 0.25 | 0.50 |
| | (SEQ ID NO: 11) | | |

Concentrations of allergens are calculated from quantitation of signature peptides (for example Analyst Bioanalytical software for LC-MS/MS), and validated by other methods including enzyme-linked immunosorbent assays (ELISA). Calculated concentrations of allergens from different samples are compared using statistical analysis, and results show good consistency among samples.

### Example 2

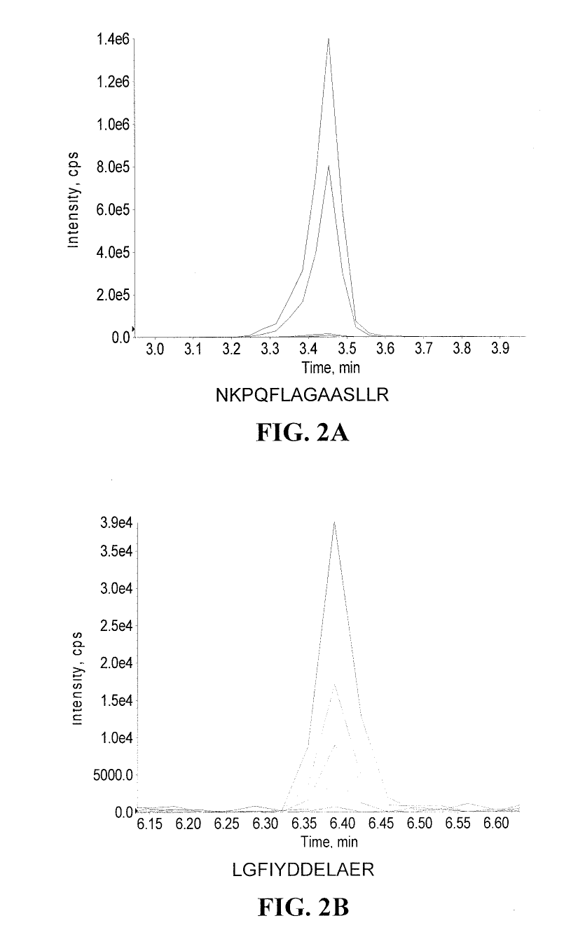

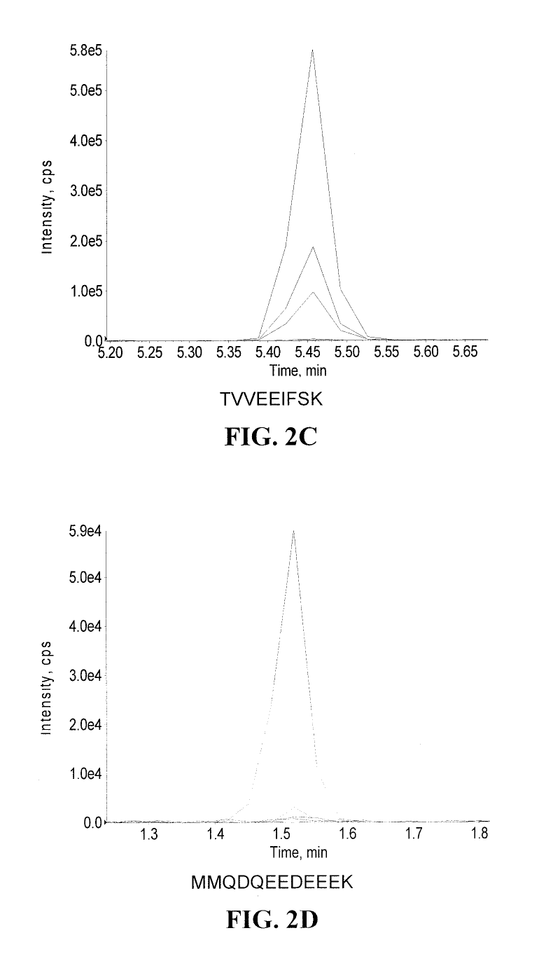

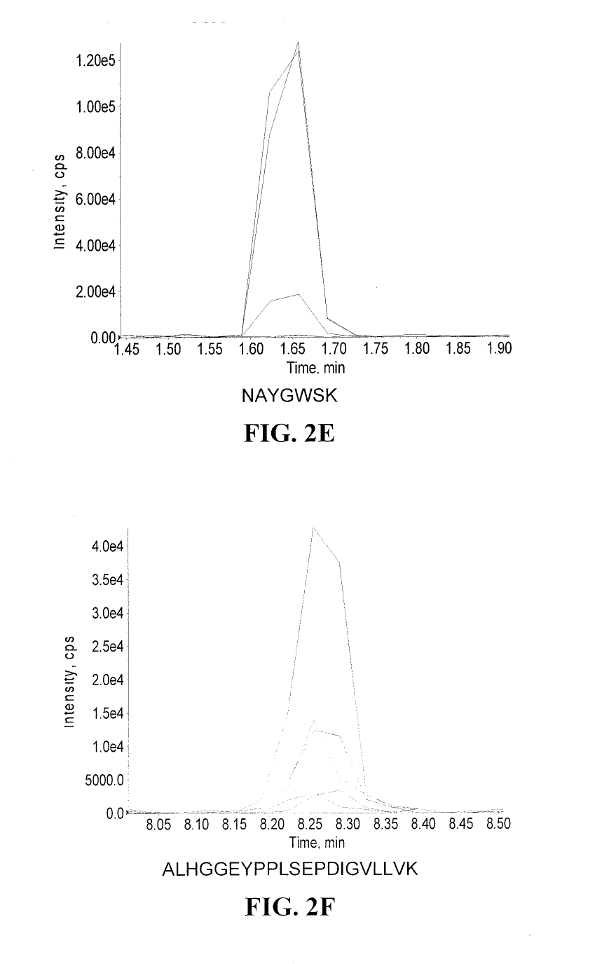

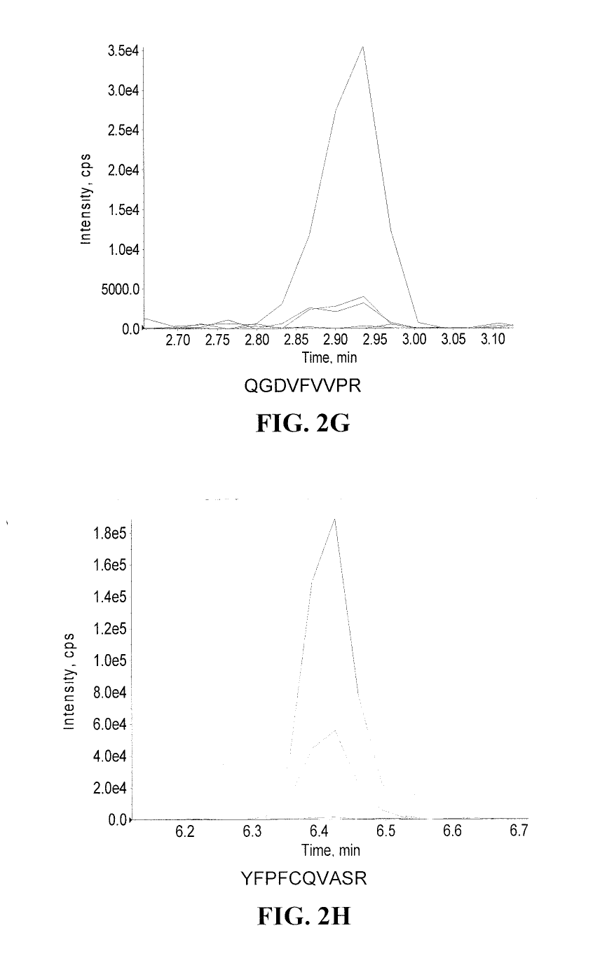

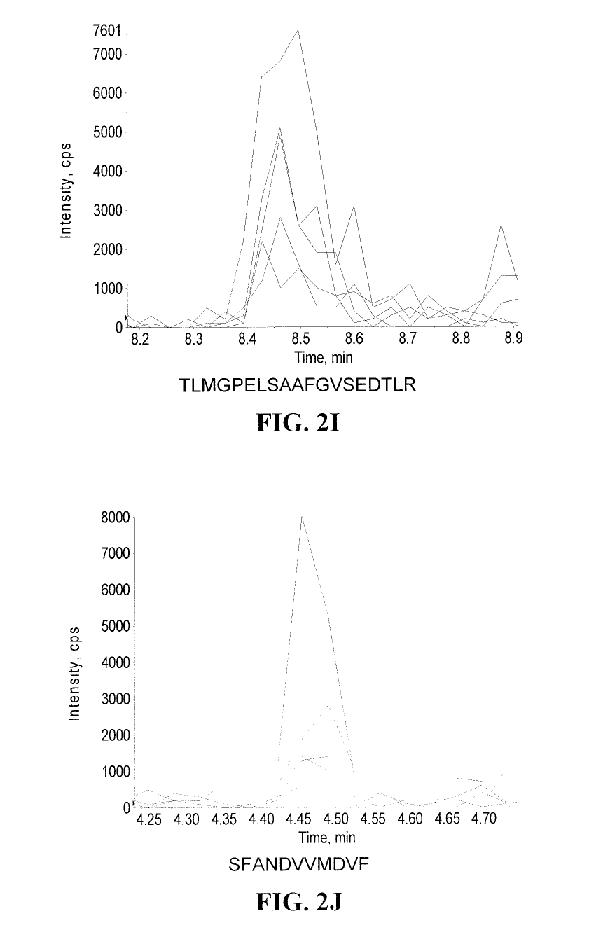

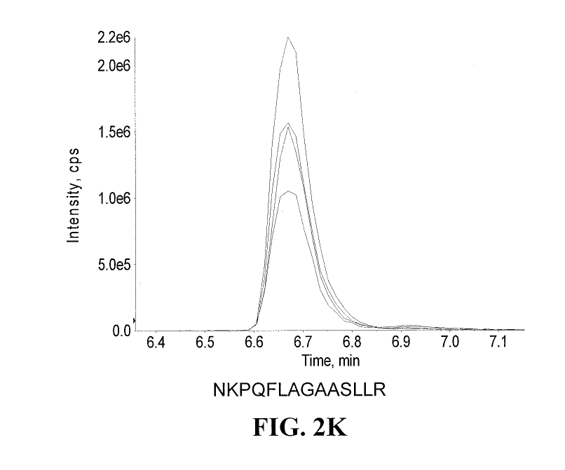

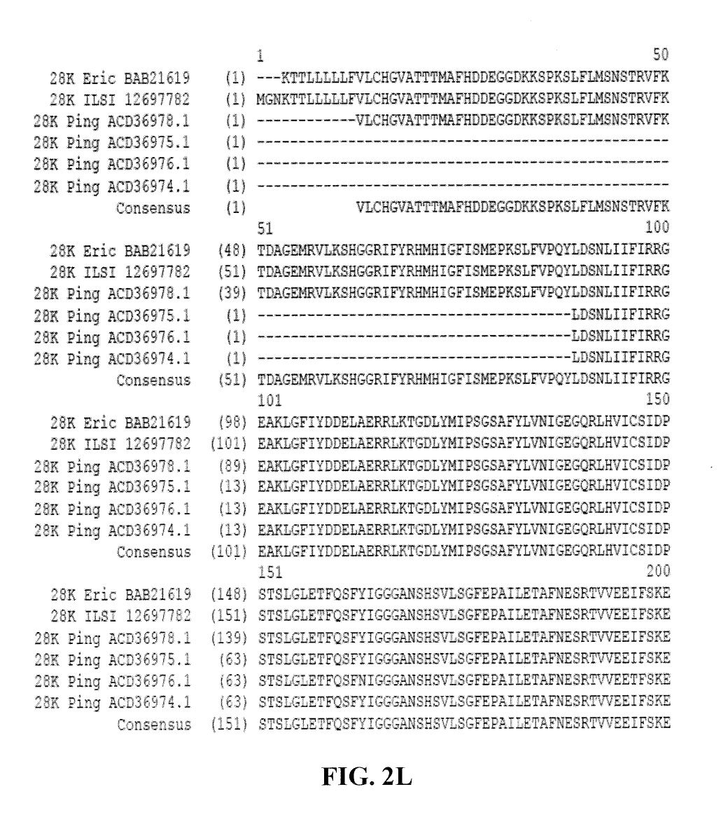

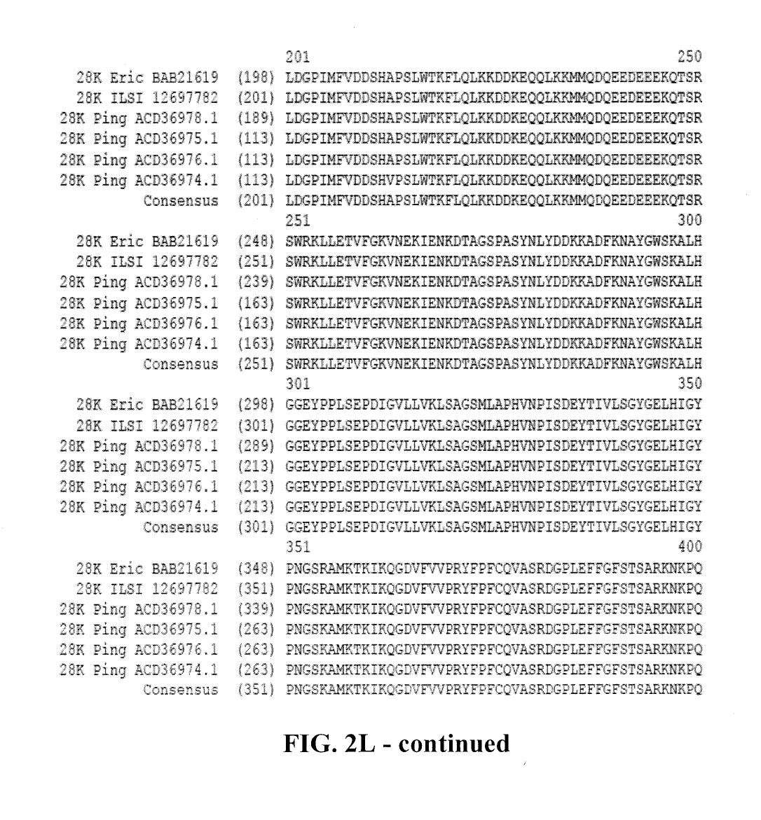

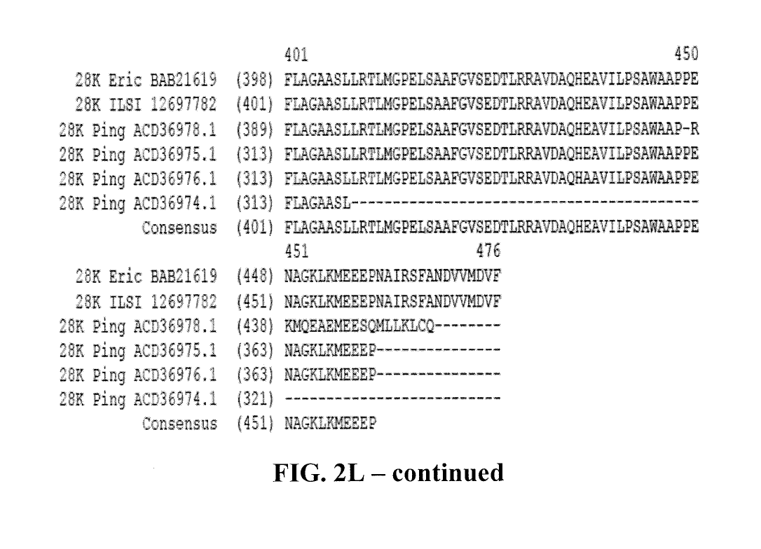

Several homologous protein sequences for Gly m Bd 28 K are identified from public databases including NCBI, Phytozome, and UniProt. Identified sequences (SEQ ID NOs: 21 and 29-33) are analyzed using bioinformatics tools to identify sequence homology and shared sequence composition among the available protein sequences (see FIG. 2L). Specifically this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure Gly m Bd 28 K itself, but also measure potential allergens which are highly homologous to Gly m Bd 28 K.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of Gly m Bd 28 K, either by itself or in combination with additional proteins in a multiplexing assay format. In this example, several signature peptides are selected from all peptide possibilities (SEQ ID NO: 9 NKPQFLAGAASLLR; SEQ ID NO: 34 LGFIYDDELAER; SEQ ID NO: 35 TVVEEIFSK; SEQ ID NO: 36 MMQDQEEDEEEK; SEQ ID NO: 37 NAYGWSK; SEQ ID NO: 38 ALHGGEYPPLSEPDIGVLLVK; SEQ ID NO: 39 QGDVFVVPR; SEQ ID NO: 40 YFPFCQVASR; SEQ ID NO: 41 TLMGPELSAAFGVSEDTLR; SEQ ID NO: 42 SFANDVVMDVF), and representative quantitation of these signature peptides are shown in FIGS. 2A-2J. A peptide standard is synthesized for SEQ ID NO: 9 NKPQFLAGAASLLR for quantitative and qualitative analyses (see FIG. 2K). Synthetic peptides can directly serve as an analytical reference standard for protein quantitation.

### Example 3

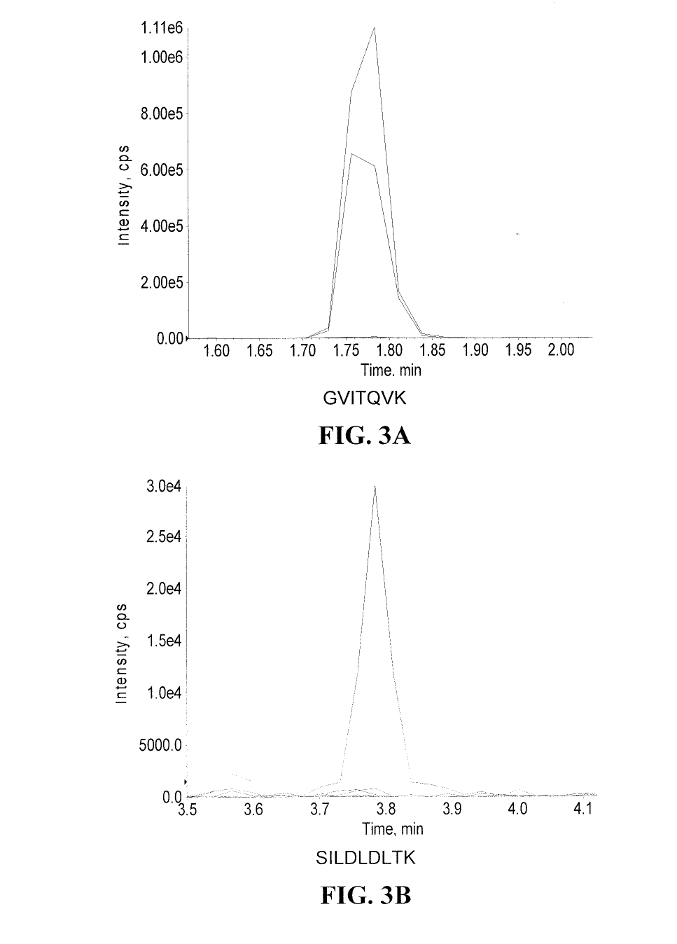

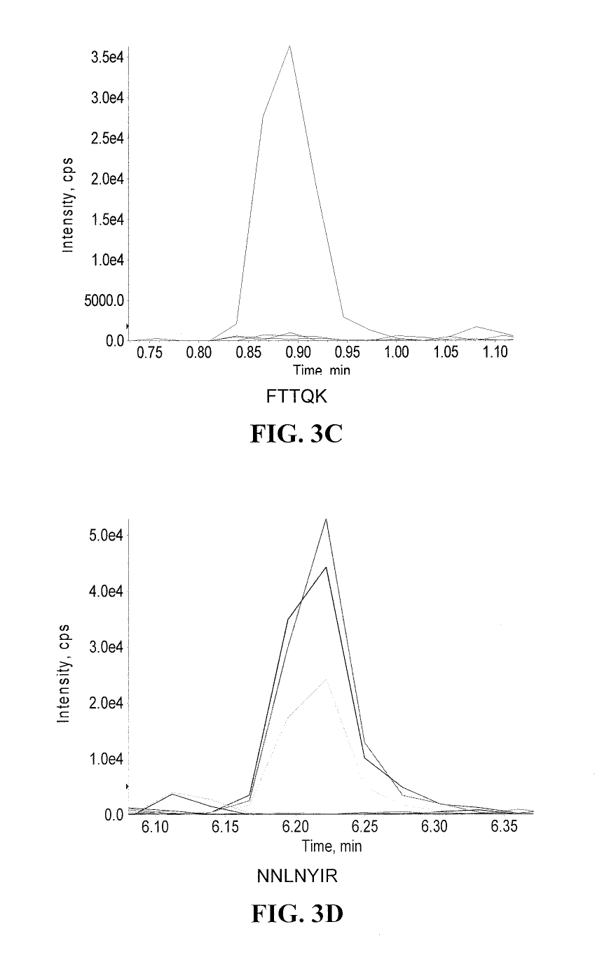

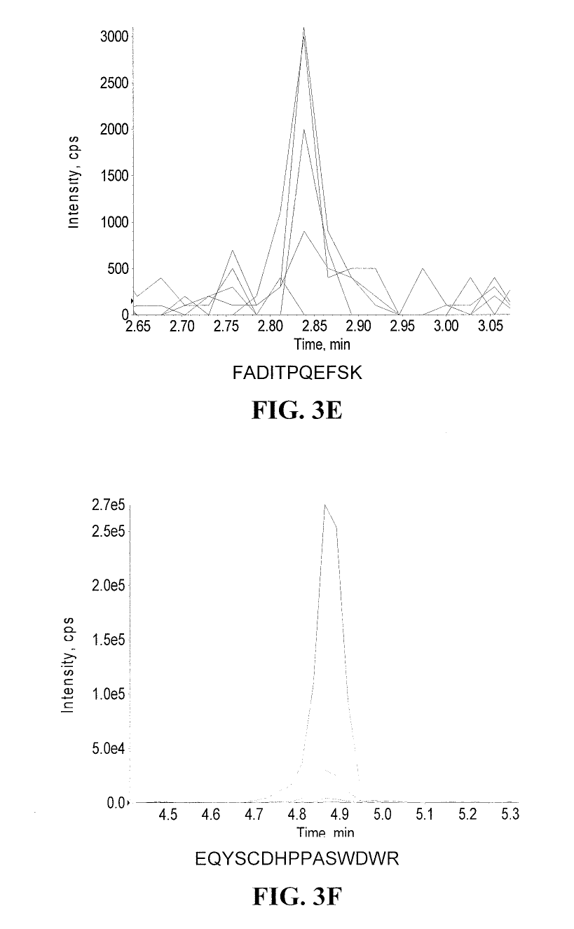

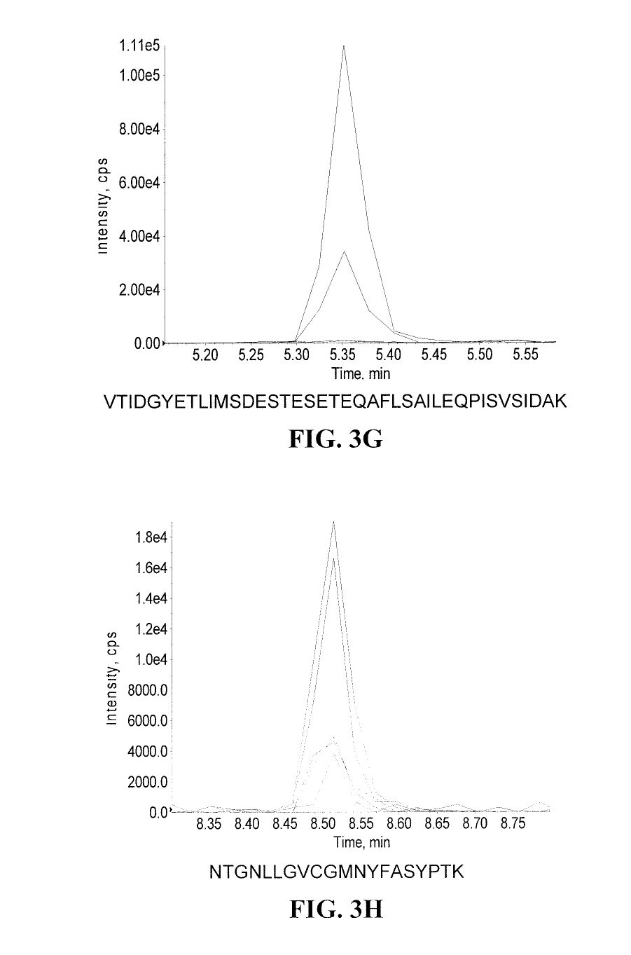

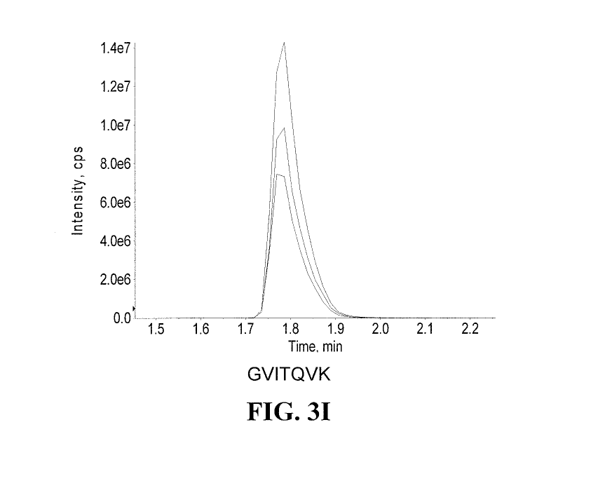

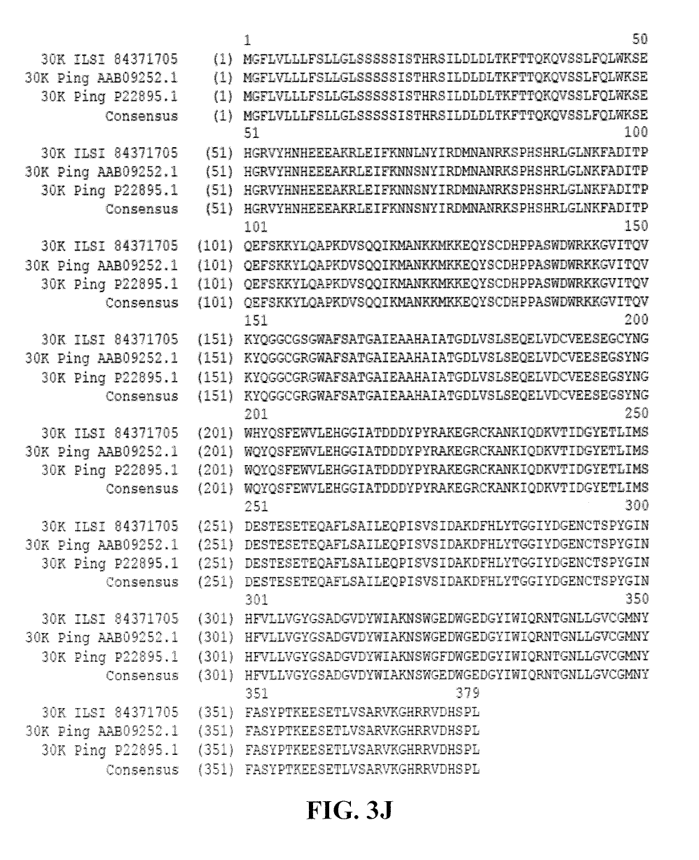

Several homologous protein sequences for Gly m Bd 30 K are identified from public databases including NCBI, Phytozome, and UniProt. Identified sequences (SEQ ID NOs: 22 and 43-44) are analyzed using bioinformatics tools to identify sequence homology and shared sequence composition among the available protein sequences (see FIG. 3J). Specifically, this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure Gly m Bd 30 K itself, but also measure potential allergens which are highly homologous to Gly m Bd 30 K.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of Gly m Bd 30 K, either by itself or in combination with additional proteins in a multiplexing assay format. In this example, several signature peptides are selected from all peptide possibilities (SEQ ID NO: 10 GVITQVK; SEQ ID NO: 45 SILDLDLTK; SEQ ID NO: 46 FTTQK; SEQ ID NO: 47 NNLNYIR; SEQ ID NO: 48 FADITPQEFSK; SEQ ID NO: 49 EQYSCDHPPASWDWR; SEQ ID NO: 50 VTIDGYETLIMSDESTESETEQAFLSAILEQPISVSIDAK; and SEQ ID NO: 51 NTGNLLGVCGMNYFASYPTK), and representative quantitation of these signature peptides are shown in FIGS. 3A-3H. A peptide standard is synthesized for SEQ ID NO: 10 GVITQVK for quantitative and qualitative analyses (see FIG. 3I). Synthetic peptides can directly serve as an analytical reference standard for protein quantitation.

### Example 4

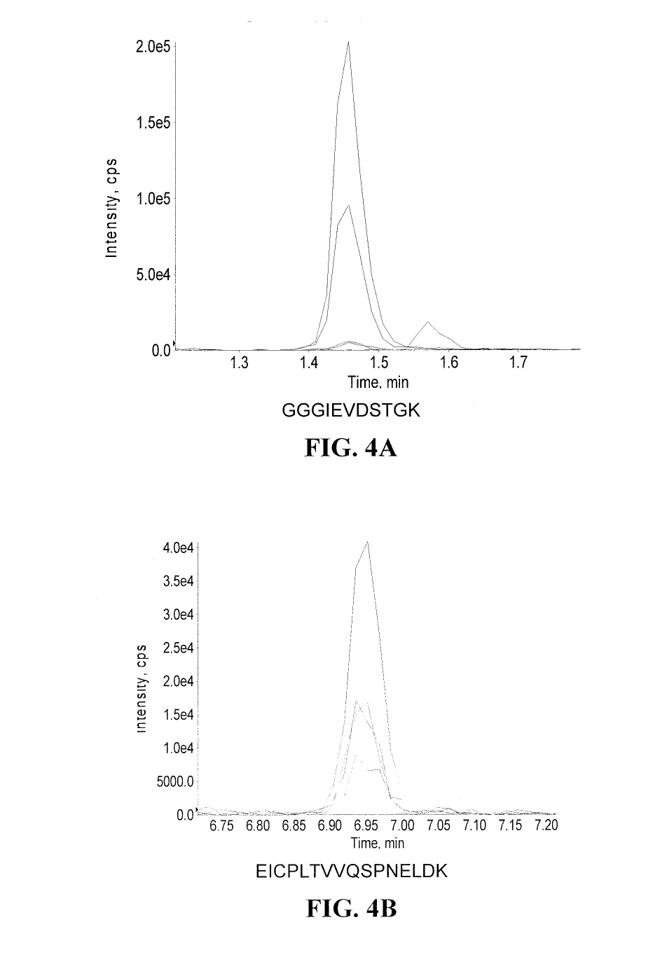

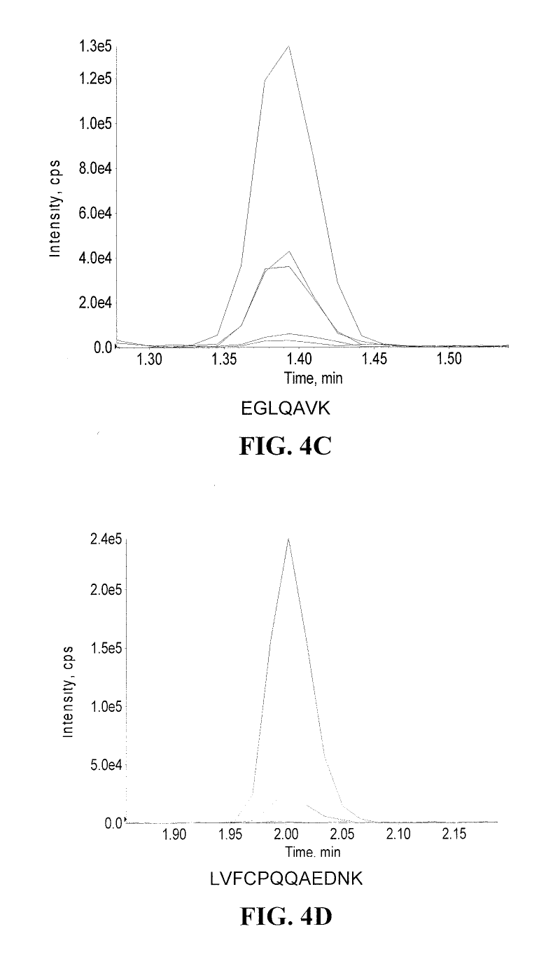

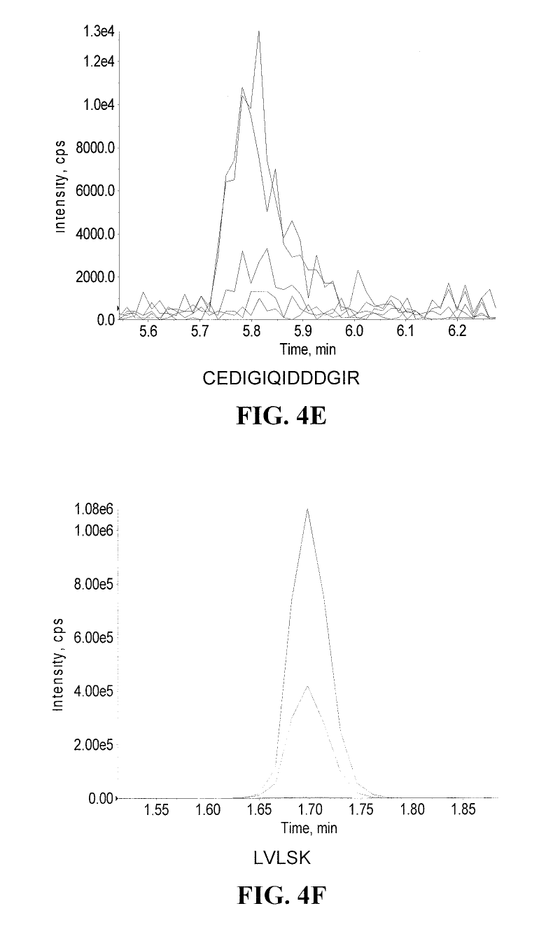

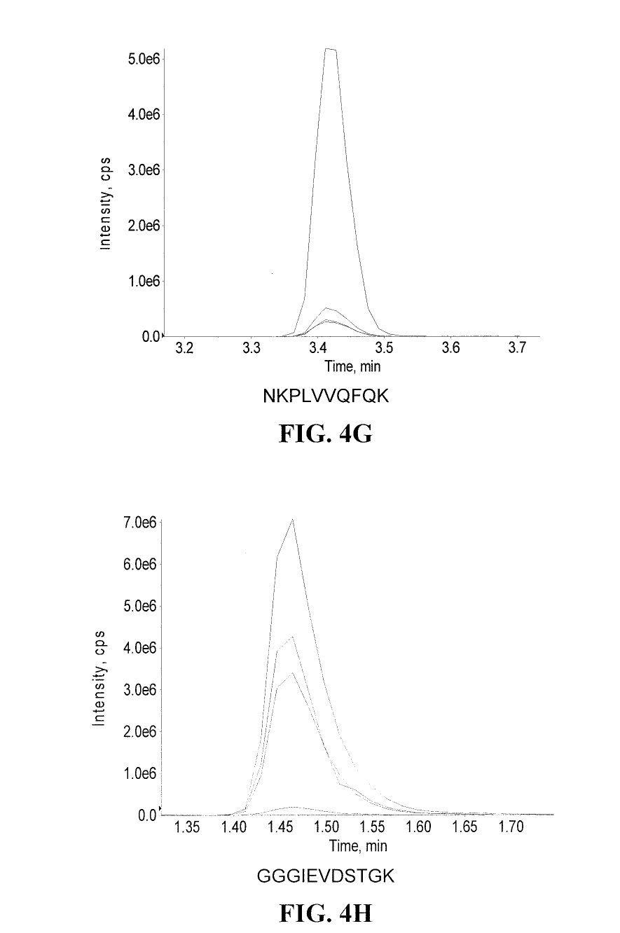

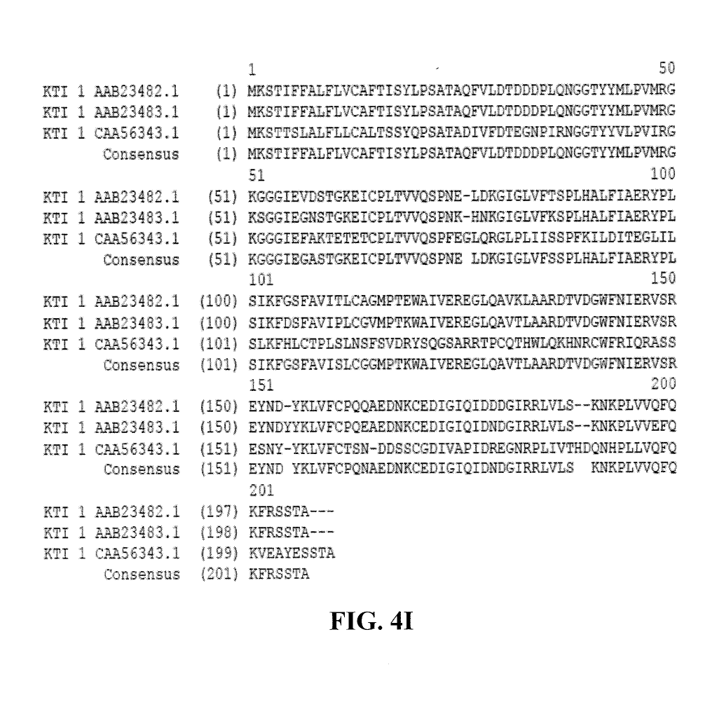

Several homologous protein sequences for Kunitz trypsin inhibitor 1 are identified from public databases including NCBI, Phytozome, and UniProt. Identified sequences (SEQ ID NOs: 23 and 53-54) are analyzed using bioinformatics tools to identify sequence homology and shared sequence composition among the available protein sequences (see FIG. 4I). Specifically this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure Kunitz trypsin inhibitor 1 itself, but also measure potential allergens which are highly homologous to Kunitz trypsin inhibitor 1.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of Kunitz trypsin inhibitor 1, either by itself or in combination with additional proteins in a multiplexing assay format. In this example, several signature peptides are selected from all peptide possibilities (SEQ ID NO: 7 GGGIEVDSTGK; SEQ ID NO: 55 EICPLTVVQSPNELDK; SEQ ID NO: 56 EGLQAVK; SEQ ID NO: 57 LVFCPQQAEDNK; SEQ ID NO: 58 CEDIGIQIDDDGIR; SEQ ID NO: 59 LVLSK; and SEQ ID NO: 60 NKPLVVQFQK), and representative quantitation of these signature peptides are shown in FIGS. 4A-4G. A peptide standard is synthesized for SEQ ID NO: 7 GGGIEVDSTGK for quantitative and qualitative analyses (see FIG. 4H). Synthetic peptides can directly serve as an analytical reference standard for protein quantitation.

### Example 5

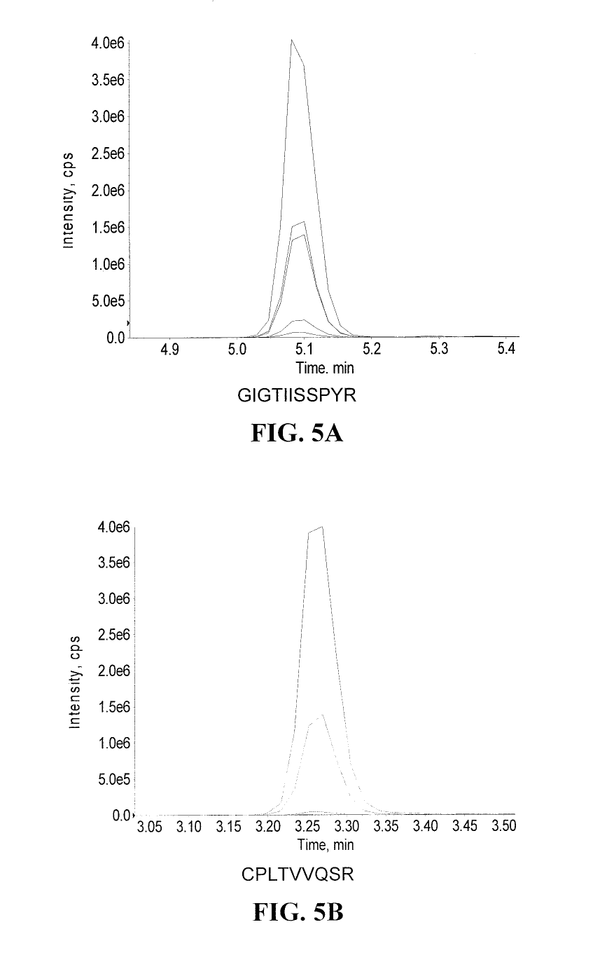

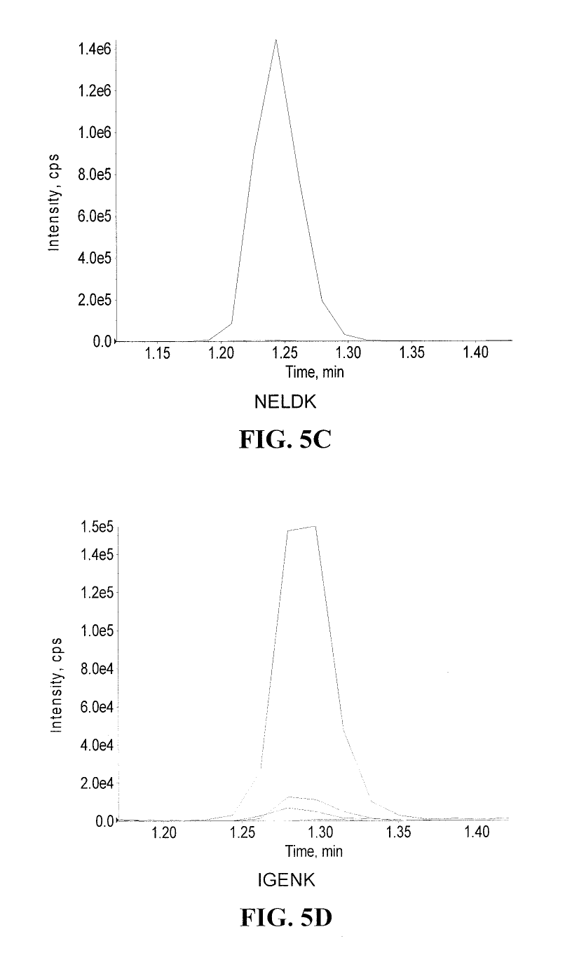

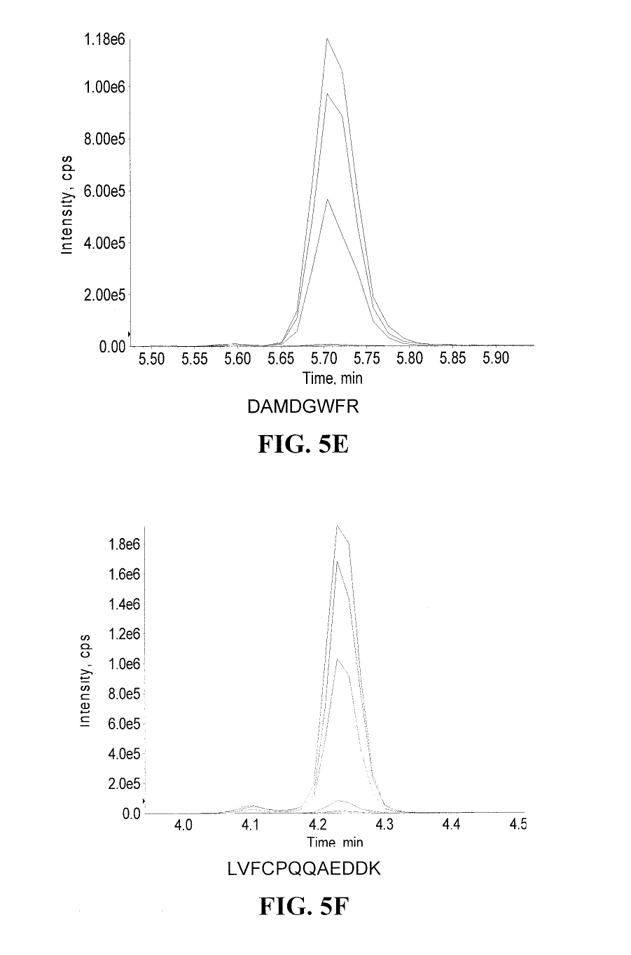

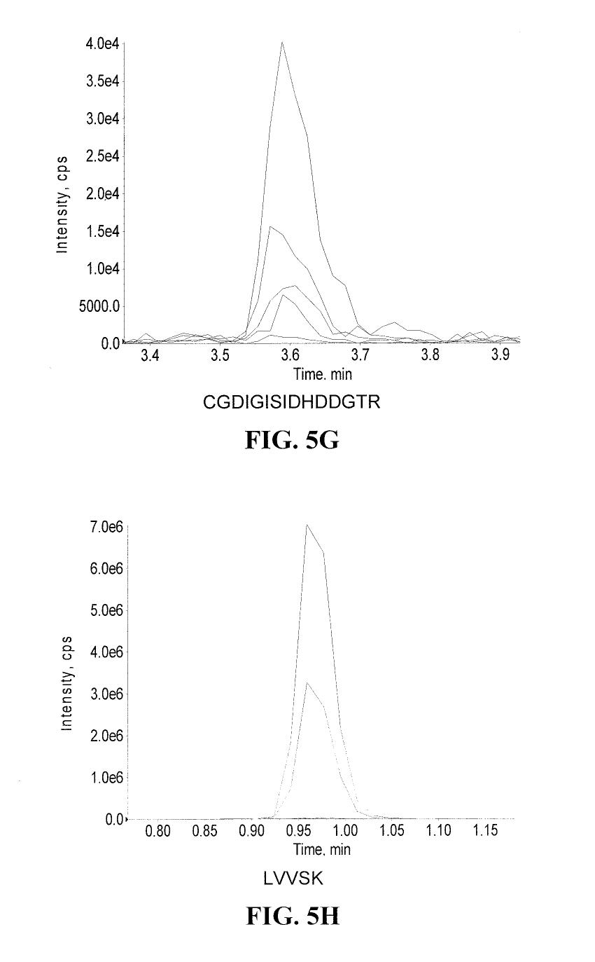

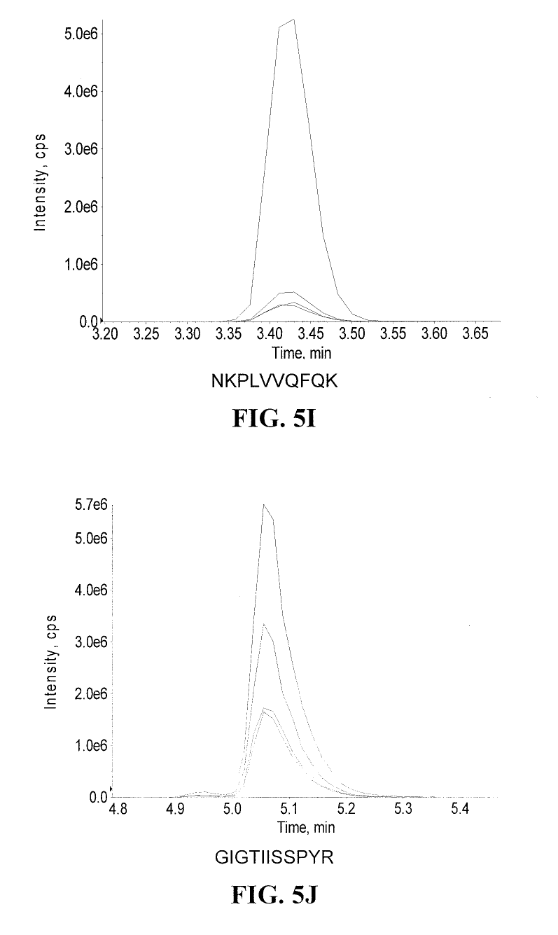

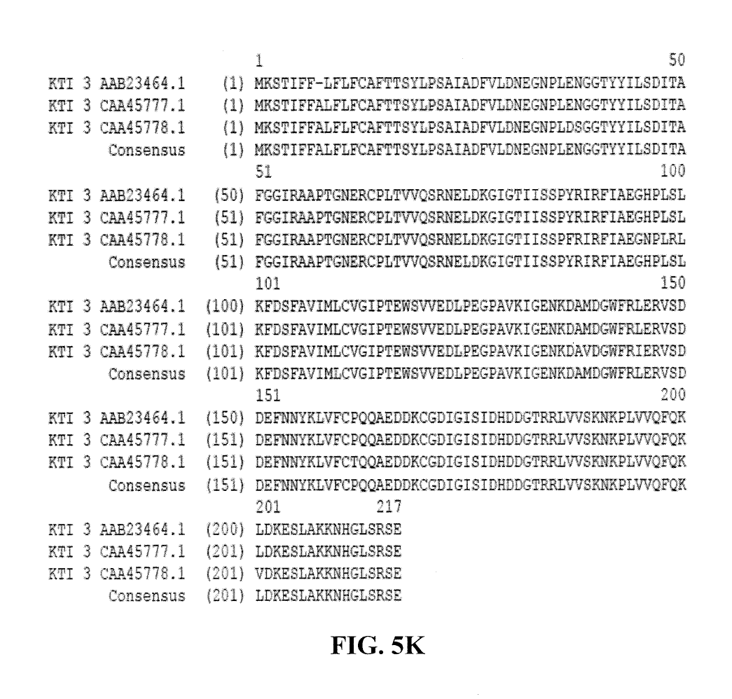

Several homologous protein sequences for Kunitz trypsin inhibitor 3 are identified from public databases including NCBI, Phytozome, and UniProt. Identified sequences (SEQ ID NOs: 24 and 62-63) are analyzed using bioinformatics tools to identify sequence homology and shared sequence composition among the available protein sequences (see FIG. 5K). Specifically, this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure Kunitz trypsin inhibitor 3 itself, but also measure potential allergens which are highly homologous to Kunitz trypsin inhibitor 3.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of Kunitz trypsin inhibitor 3, either by itself or in combination with additional proteins in a multiplexing assay format. In this example, several signature peptides are selected from all peptide possibilities (SEQ ID NO: 8 GIGTIISSPYR; SEQ ID NO: 64 CPLTVVQSR; SEQ ID NO: 65 NELDK; SEQ ID NO: 66 IGENK; SEQ ID NO: 67 DAMDGWFR; SEQ ID 68 LVFCPQQAEDDK; SEQ ID NO: 69 CGDIGISIDHDDGTR; SEQ ID NO: 70 LVVSK; and SEQ ID NO: 71 NKPLVVQFQK), and representative quantitation of these signature peptides are shown in FIGS. 5A-5I. A peptide standard is synthesized for SEQ ID NO: 8 GIGTIISSPYR for quantitative and qualitative analyses (see FIG. 5J). Synthetic peptides can directly serve as an analytical reference standard for protein quantitation.

### Example 6

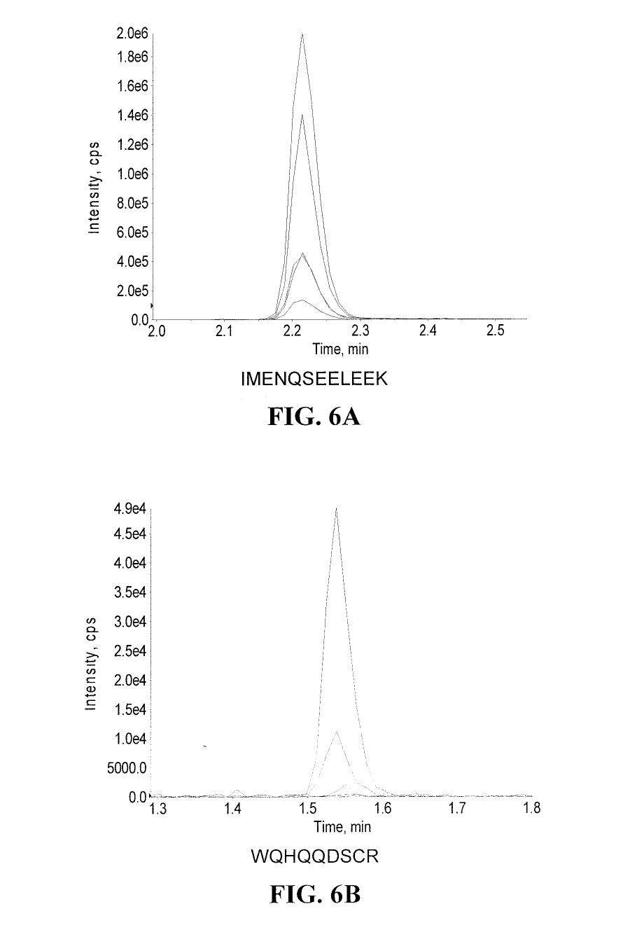

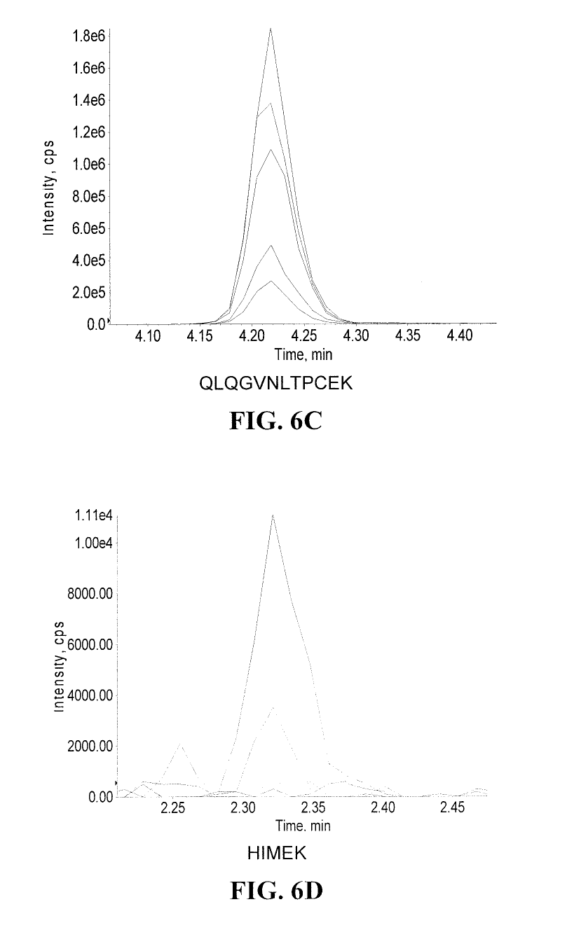

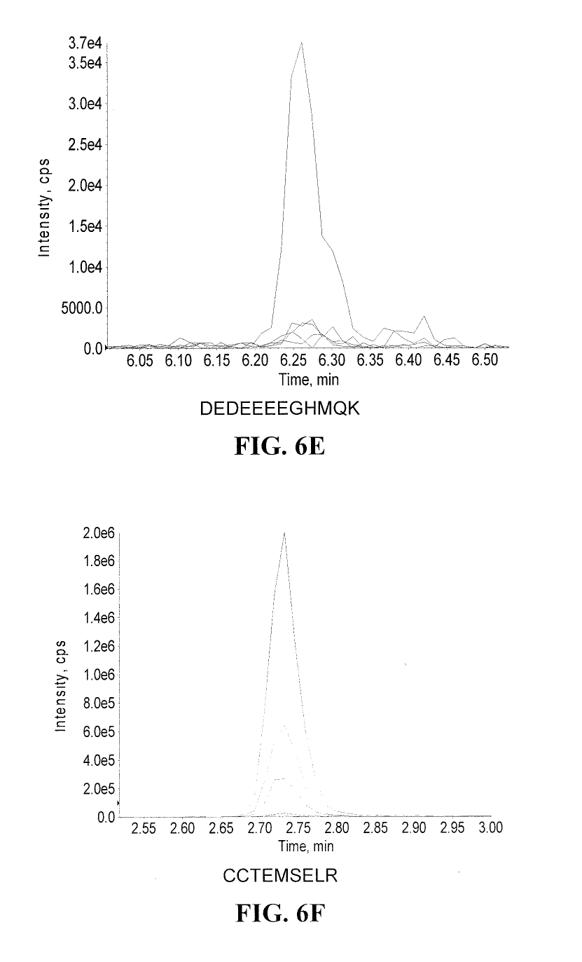

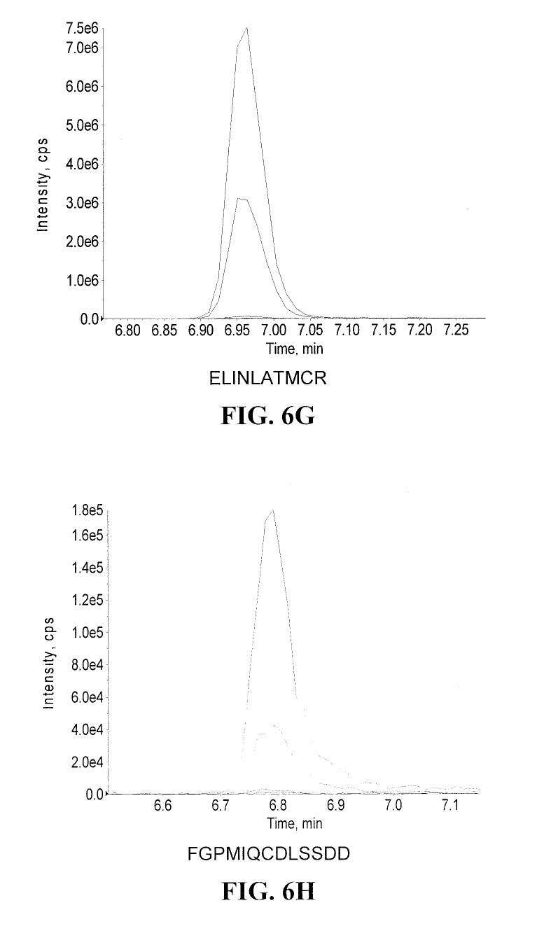

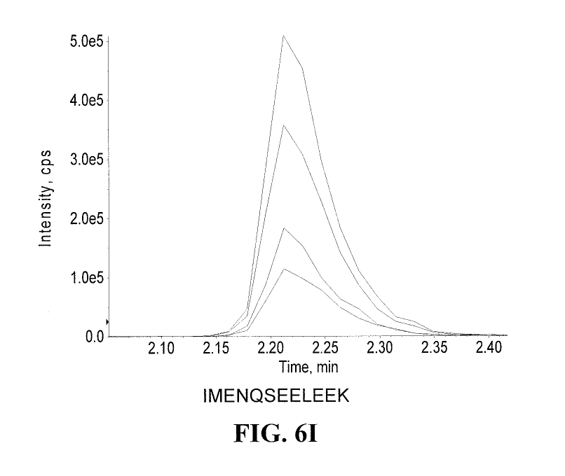

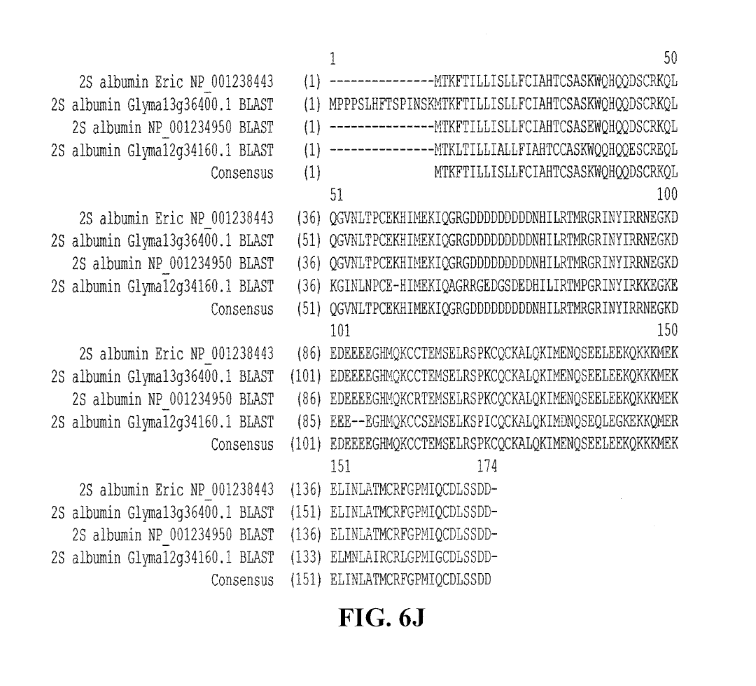

Several homologous protein sequences for Gly m 8 (2S albumin) are identified from public databases including NCBI, Phytozome, and UniProt. Identified sequences (SEQ ID NOs: 25 and 72-74) are analyzed using bioinformatics tools to identify sequence homology and shared sequence composition among the available protein sequences (see FIG. 6J). Specifically this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure Gly m 8 (2S albumin) itself, but also measure potential allergens which are highly homologous to Gly m 8 (2S albumin).

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of Gly m 8 (2S albumin), either by itself or in combination with additional proteins in a multiplexing assay format. In this example, several signature peptides are selected from all peptide possibilities (SEQ ID NO: 11 IMENQSEELEEK; SEQ ID NO: 75 WQHQQDSCR; SEQ ID NO: 76 QLQGVNLTPCEK; SEQ ID NO: 77 HIMEK; SEQ ID NO: 78 DEDEEEEGHMQK; SEQ ID NO: 79 CCTEMSELR; SEQ ID NO: 80 ELINLATMCR; and SEQ ID NO: 81 FGPMIQCDLSSDD), and representative quantitation of these signature peptides are shown in FIGS. 6A-6H. A peptide standard is synthesized for SEQ ID NO: 11 IMENQSEELEEK for quantitative and qualitative analyses (see FIG. 61). Synthetic peptides can directly serve as an analytical reference standard for protein quantitation.

### Example 7

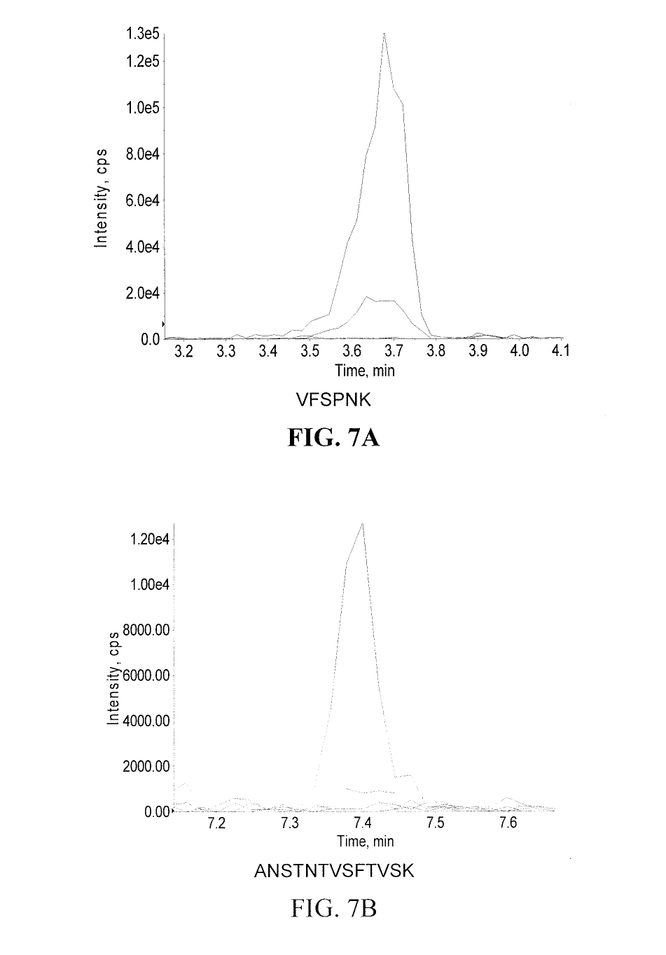

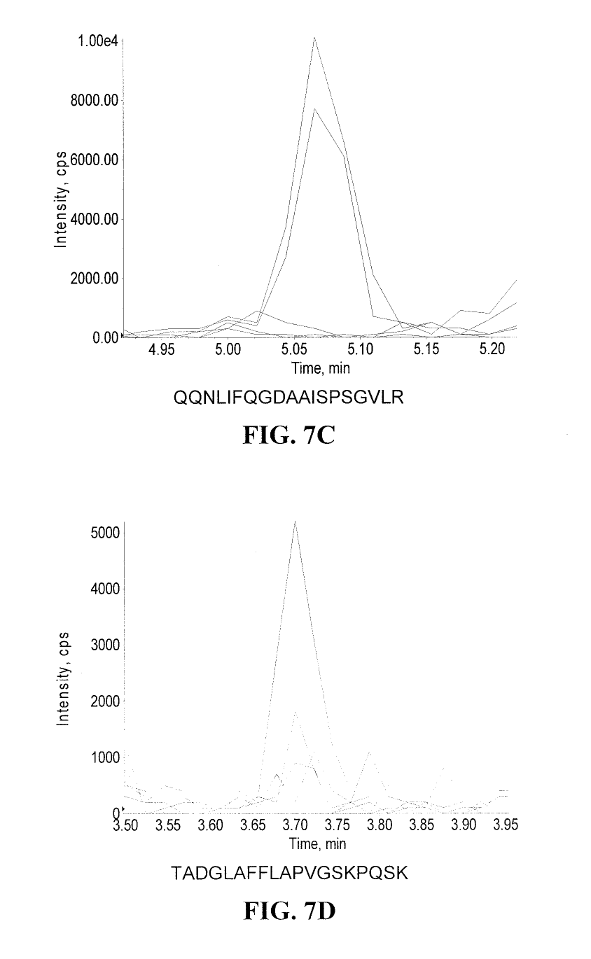

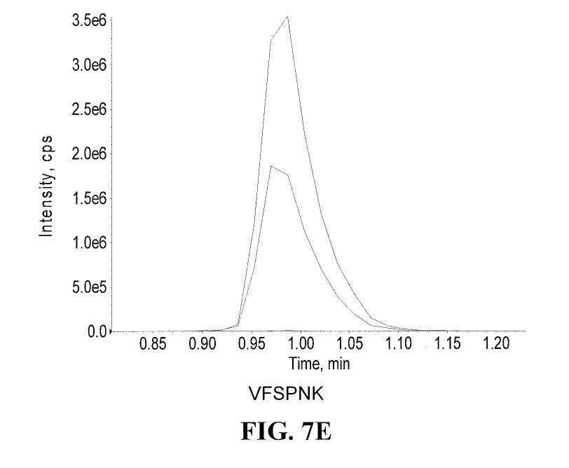

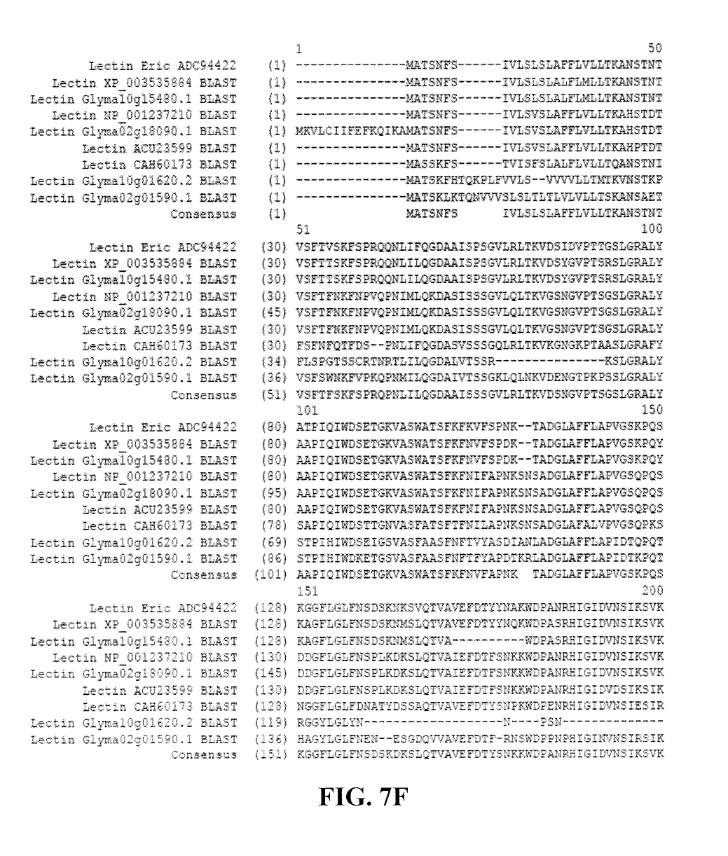

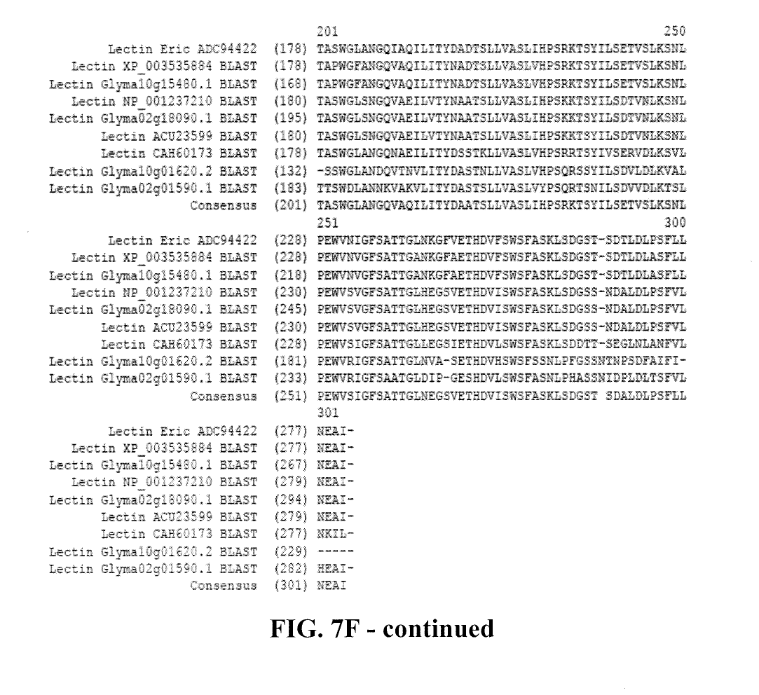

Several homologous protein sequences for Lectin are identified from public databases including NCBI, Phytozome, and UniProt. Identified sequences (SEQ ID NOs: 26 and 83-90) are analyzed using bioinformatics tools to identify sequence homology and shared sequence composition among the available protein sequences (see FIG. 7F). Specifically this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure lectin itself, but also measure potential allergens which are highly homologous to Lectin.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of lectin, either by itself or in combination with additional proteins in a multiplexing assay format. In this example, several signature peptides are selected from all peptide possibilities (SEQ ID NO: 91 VFSPNK; SEQ ID NO: 92 ANSTNTVSFTVSK; SEQ ID NO: 93 QQNLIFQGDAAISPSGVLR; and SEQ ID NO: 94 TADGLAFFLAPVGSKPQSK), and representative quantitation of these signature peptides are shown in FIGS. 7A-7D. A peptide standard is synthesized for SEQ ID NO: 91 VFSPNK for quantitative and qualitative analyses (see FIG. 7E). Synthetic peptides can directly serve as an analytical reference standard for protein quantitation.

### Example 8

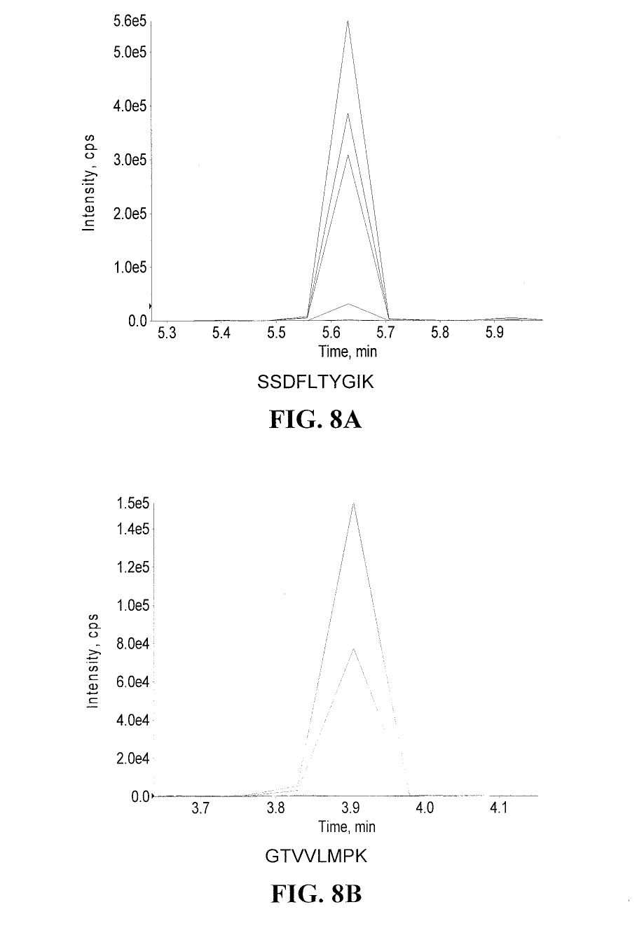

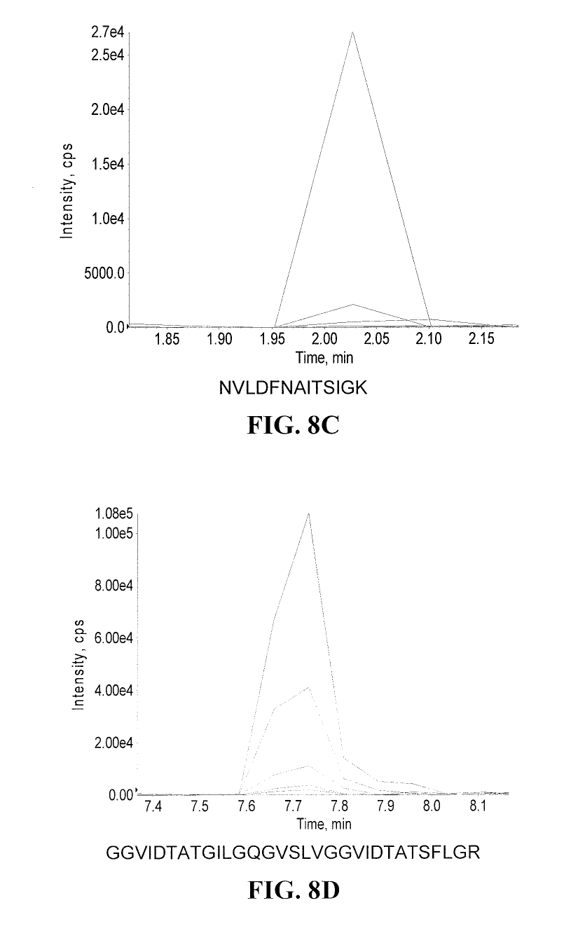

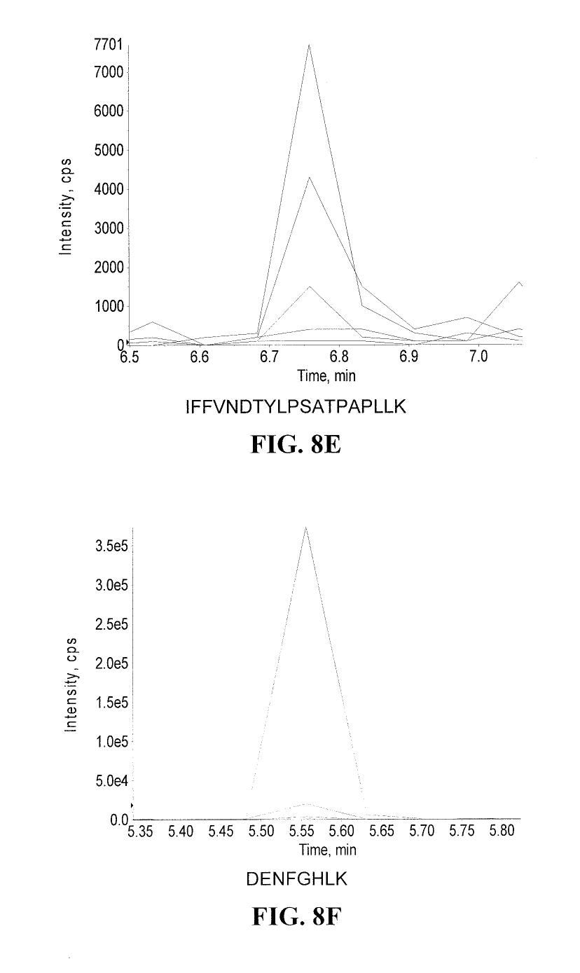

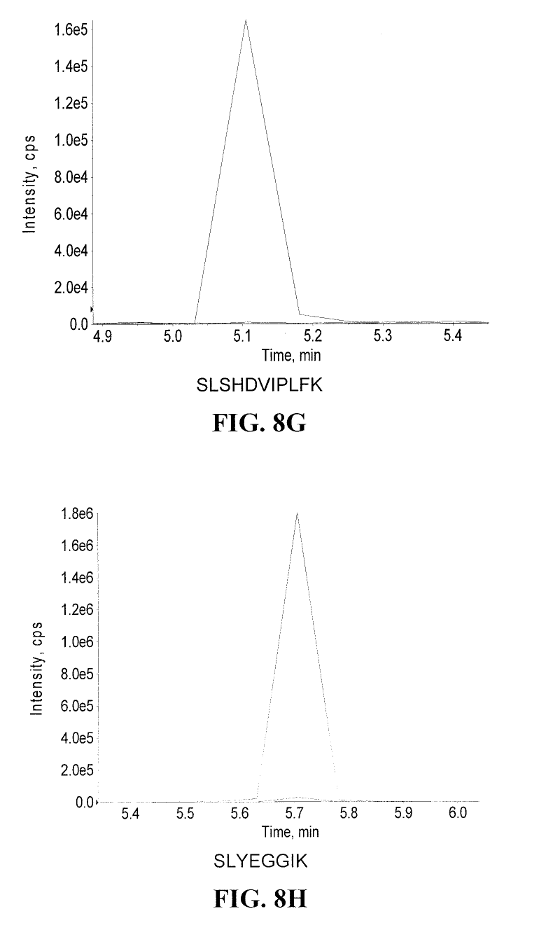

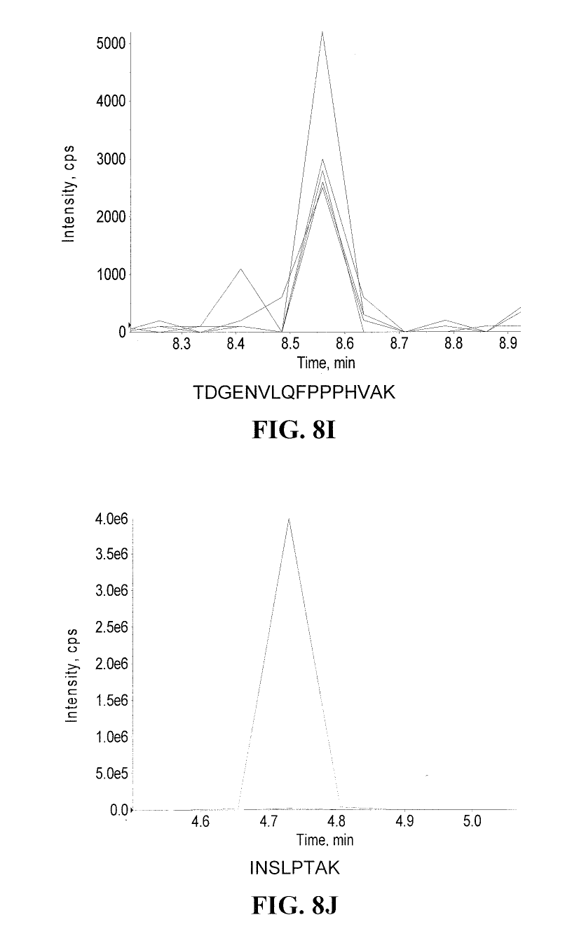

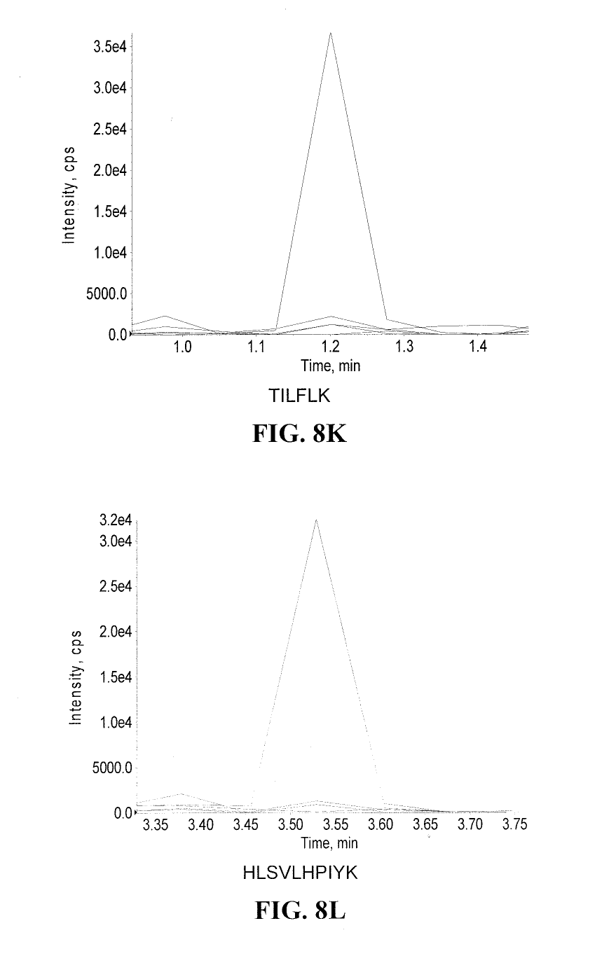

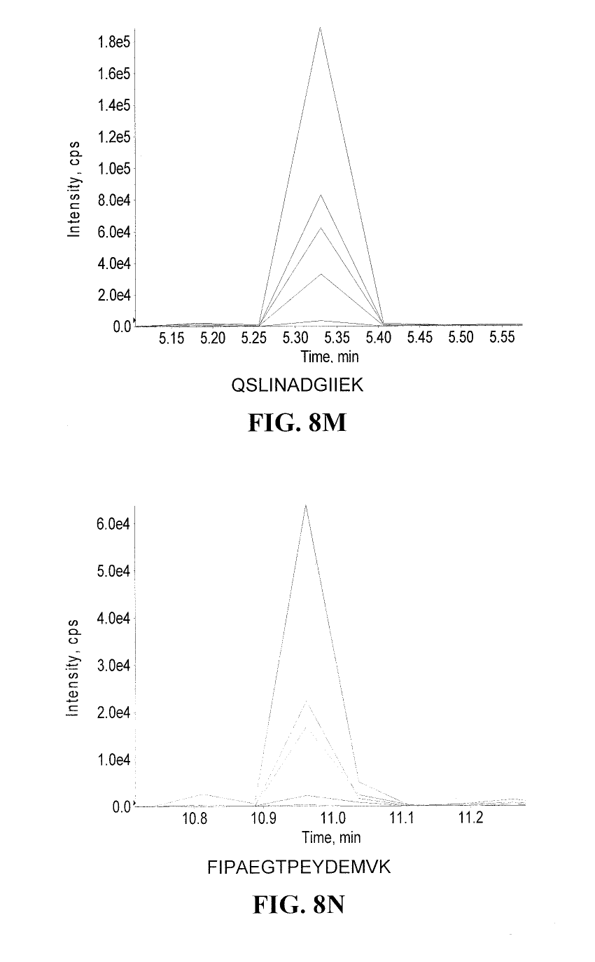

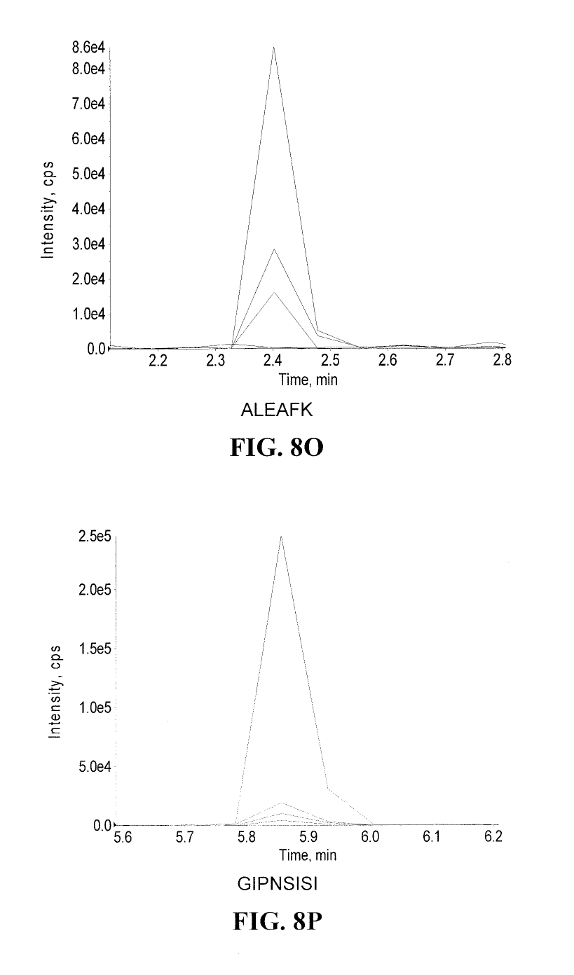

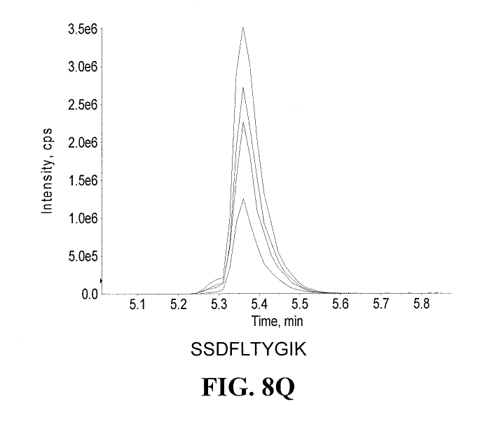

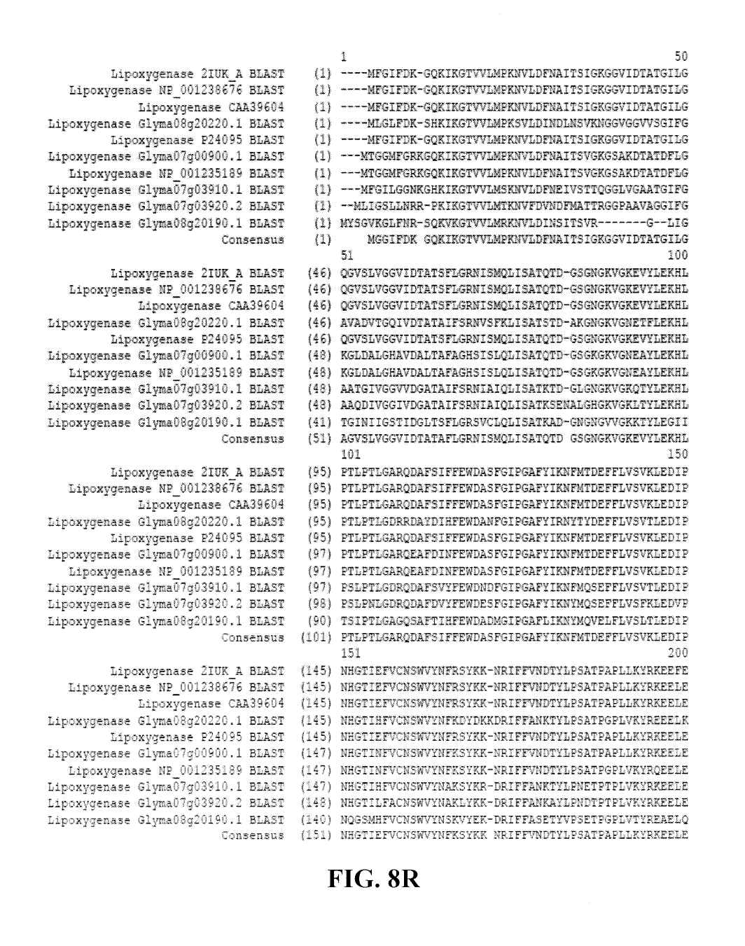

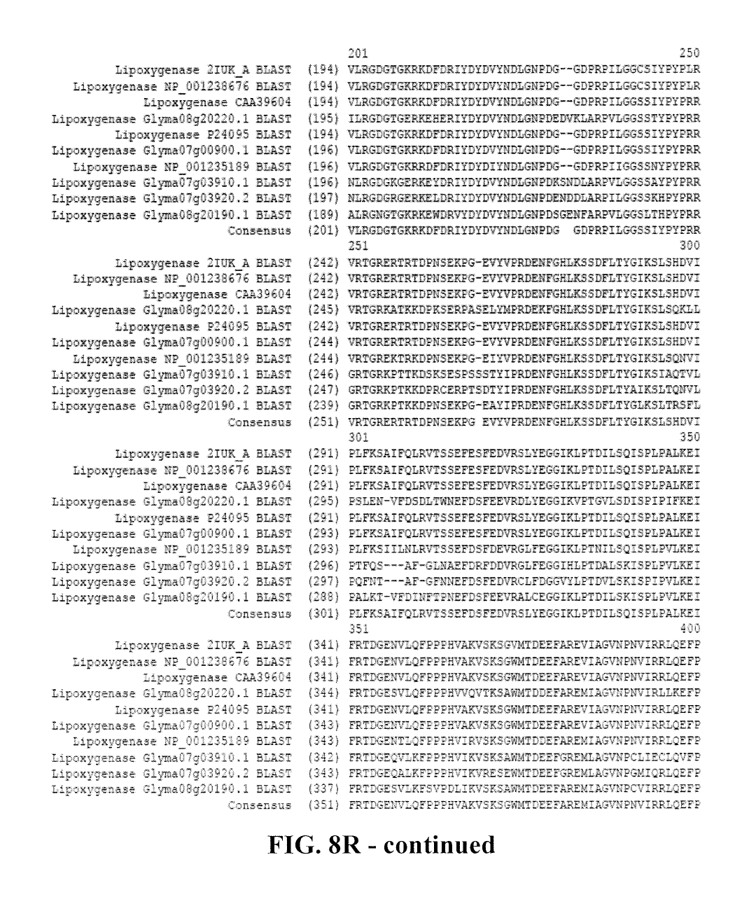

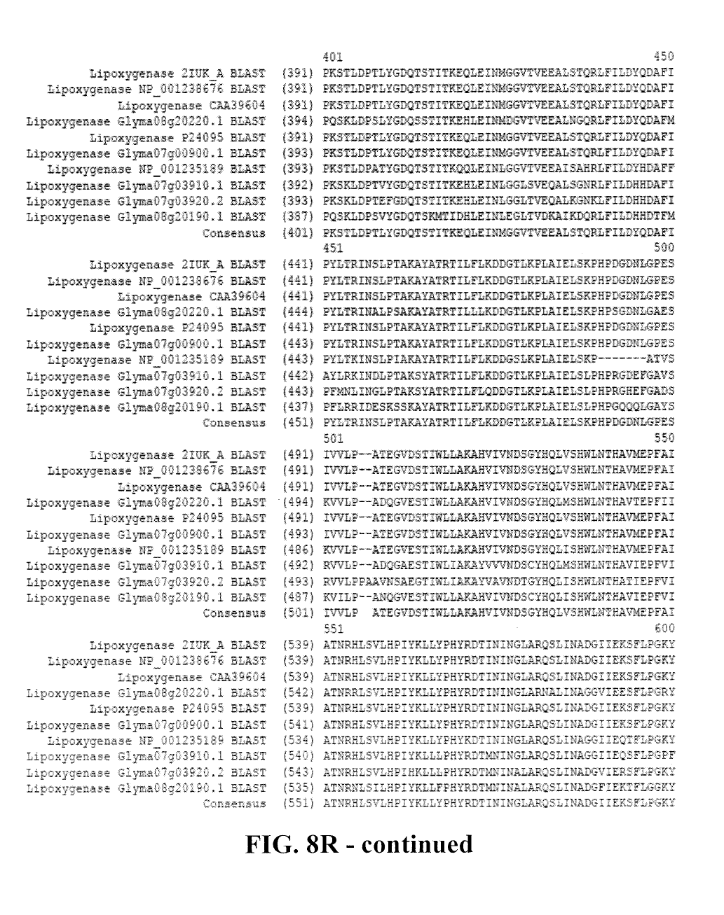

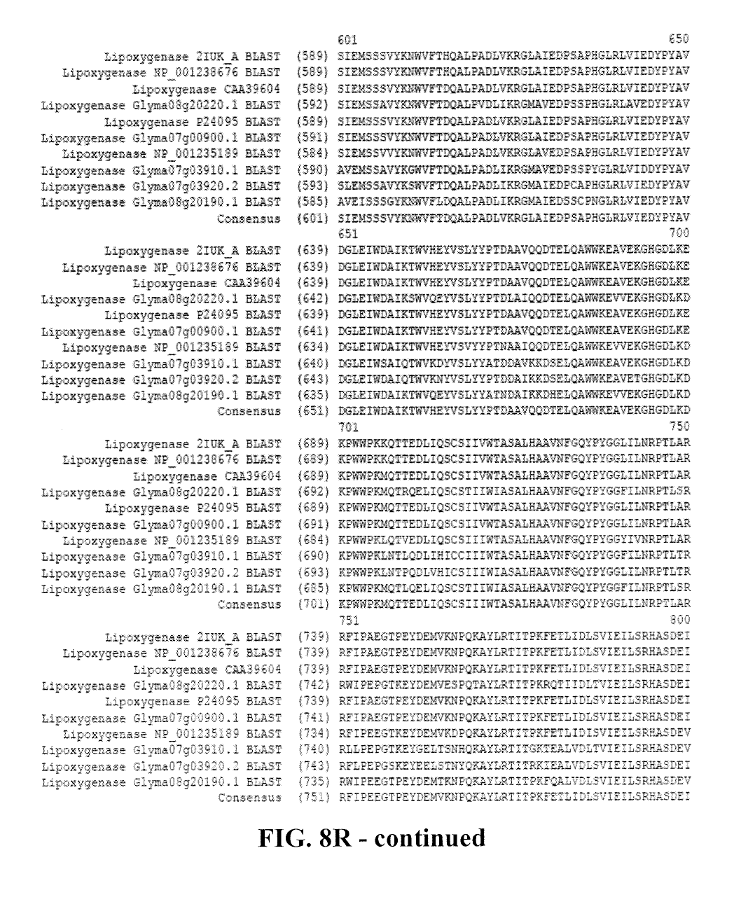

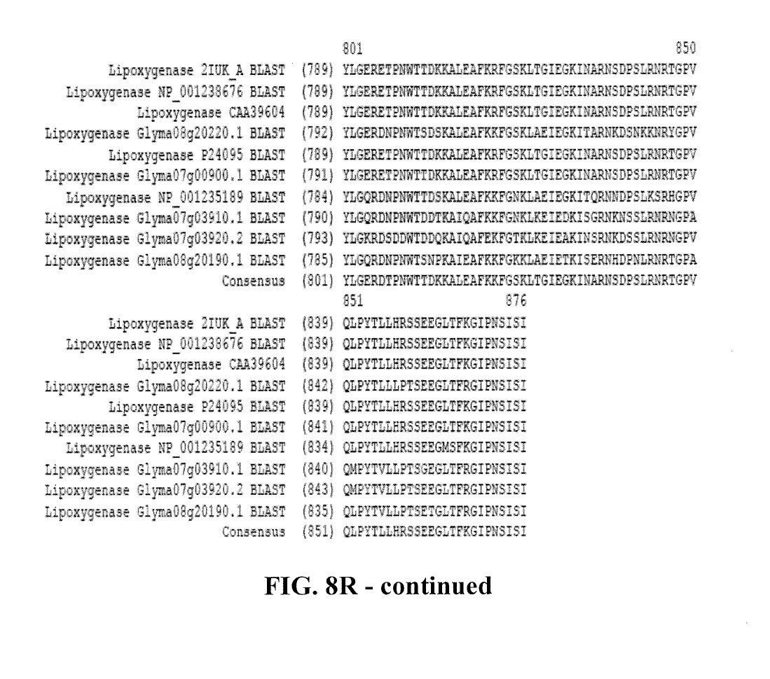

Several homologous protein sequences for lipoxygenase are identified from public databases including NCBI, Phytozome, and UniProt. Identified sequences (SEQ ID NOs: 27 and 96-104) are analyzed using bioinformatics tools to identify sequence homology and shared sequence composition among the available protein sequences (see FIG. 8R). Specifically this involved the use of Vector NTI Align X alignment tool which performs a CLUSTAL W type alignment. From this analysis, a consensus sequence and/or representative sequence can be determined.

Once the consensus sequence and/or representative sequence is chosen or determined, it is digested in silico to generate candidate signature peptide fragments to be detected and measured by LC-MS. According to the unique approaches provided herein, signature peptides are selected based on the degree of conservation among the available protein sequences, such that the selected signature peptide can be used to quantify all or as many protein isoforms as possible among the identified protein sequences found in the public sequence databases. As a result, quantitation of selected signature peptides can not only measure lipoxygenase itself, but also measure potential allergens which are highly homologous to lipoxygenase.

Soybean seed samples are ground to a fine powder, defatted twice with hexane, and extracted with suitable assay buffer (for example 5 M urea, 2 M thiourea, 50 mM Tris (pH 8.0), 65 mM DTT). The samples are sonicated in buffer to extract proteins. The extracted proteins are diluted, denatured, and then proteolytically digested by adding trypsin protease and incubating at 37° C. for 15-20 hours. The digestion reactions are acidified with formic acid (pH=1-2) and are analyzed using LC-MS/MS.

The selected signature peptides can be used for both qualitative and quantitative analysis of lipoxygenase, either by itself or in combination with additional proteins in a multiplexing assay format. In this example, several signature peptides are selected from all peptide possibilities (SEQ ID NO: 105 SSDFLTYGIK; SEQ ID NO: 106 GTVVLMPK; SEQ ID NO: 107 NVLDFNAITSIGK; SEQ ID NO: 108 GGVIDTATGILGQGVSLVGGVIDTATSFLGR; SEQ ID NO: 109 IFF VNDTYLPSATPAPLLK; SEQ ID NO: 110 DENFGHLK; SEQ ID NO: 111 SLSHDVIPLFK; SEQ ID NO: 112 SLYEGGIK; SEQ ID NO: 113 TDGENVLQFPPPHVAK; SEQ ID NO: 114 INSLPTAK; SEQ ID NO: 115 TILFLK; SEQ ID NO: 116 HLSVLHPIYK; SEQ ID NO: 117 QSLINADGIIEK; SEQ ID NO: 118 FIPAEGTPEYDEMVK; SEQ ID NO: 119 ALEAFK; and SEQ ID NO: 120 GIPNSISI), and representative quantitation of these signature peptides are shown in FIGS. 8A-8P. A peptide standard is synthesized for SEQ ID NO: 105 SSDFLTYGIK for quantitative and qualitative analyses (see FIG. 8Q). Synthetic peptides can directly serve as an analytical reference standard for protein quantitation.

What is claimed is:

1. A method of selecting candidate signature peptide for quantitation of known allergen and potential allergens from a plant-based sample, comprising:
(a) identifying potential allergens based on homology to at least one known allergen protein sequence;
(b) performing sequence alignment of the at least one known allergen and potential allergens identified in step (a);
(c) selecting a consensus sequence or representative sequence based on the sequence alignment;
(d) determining a plural of candidate signature peptides based on conservative regions or domains from the sequence alignment and in silico digestion data of the consensus sequence or representative sequence selected in Step (c); and
(e) quantitating the amount of the at least one known allergen and potential allergens in the plant-based sample based on measurements of the signature peptides.

2. The method of claim 1, wherein the quantitating step uses a column chromatography and mass spectrometry.

3. The method of claim 1, wherein the quantitating step comprises measuring the plural of candidate signature peptides using high resolution accurate mass spectrometry (HRAM MS).

4. The method of claim 1, wherein the quantitating step comprises calculating corresponding peak heights or peak areas of the candidate signature peptides from mass spectrometry.

5. The method of claim 1, wherein the quantitating step comprises comparing data from high fragmentation mode and low fragmentation mode from mass spectrometry.

6. The method of claim 1, wherein the at least one known allergen comprises Gly m Bd 28 K, Gly m Bd 30 K, Kunitz trypsin inhibitor 1, Kunitz trypsin inhibitor 3, Gly m 8 (2S albumin), Lectin, or lipoxygenase.

7. The method of claim 1, wherein the potential allergens comprise at least one sequence selected from the group consisting of:
(a) SEQ ID NOs: 21 and 29-33 for Gly m Bd 28 K;
(b) SEQ ID NOs: 22 and 43-44 for Gly m Bd 30 K;
(c) SEQ ID NOs: 23 and 53-54 for Kunitz trypsin inhibitor 1;
(d) SEQ ID NOs: 24 and 62-63 for Kunitz trypsin inhibitor 3;
(e) SEQ ID NOs: 25 and 72-74 for Gly m 8 (2S albumin);
(f) SEQ ID NOs: 26 and 83-90 for Lectin; and
(g) SEQ ID NOs: 27 and 96-104 for lipoxygenase.

8. The method of claim 1, wherein the candidate signature peptides comprise at least one sequence selected from the group consisting of:
(a) SEQ ID NOs: 9 and 34-42 for Gly m Bd 28 K;
(b) SEQ ID NOs: 10 and 45-51 for Gly m Bd 30 K;
(c) SEQ ID NOs: 7 and 55-60 for Kunitz trypsin inhibitor 1;
(d) SEQ ID NOs: 8 and 64-71 for Kunitz trypsin inhibitor 3;
(e) SEQ ID NOs: 11 and 75-81 for Gly m 8 (2S albumin);
(f) SEQ ID NOs: 91-94 for Lectin; and
(g) SEQ ID NOs: 105-120 for lipoxygenase.

9. The method of claim 1, wherein the plant-based sample comprises a soybean seed or part of a soybean seed.

10. A system for quantitating one or more protein of interest with known amino acid sequence in a plant-based sample, the system comprising:
(a) a high-throughput means for extracting proteins from a plant-based sample;
(b) a process module for digesting extracted proteins with at least one protease;
(c) a separation module for separating peptides in a single step;
(d) a selection module for selecting a plural of signature peptides for at least one known allergen and potential allergens; and
(e) a mass spectrometry for measuring the plural of signature peptides.

11. The system of claim 10, wherein the separation module comprises a column chromatography.

12. The system of claim 11, wherein the column chromatography comprises a liquid column chromatography.

13. The system of claim 10, wherein the mass spectrometry comprises a high resolution accurate mass spectrometry (HRAM MS).

14. The system of claim 10, wherein the selection module uses a method according to claim 1.

15. The system of claim 10, wherein the at least one known allergen comprises Gly m Bd 28 K, Gly m Bd 30 K, Kunitz trypsin inhibitor 1, Kunitz trypsin inhibitor 3, Gly m 8 (2S albumin), Lectin, or lipoxygenase.

16. The system of claim 10, wherein the potential allergens comprise at least one sequence selected from the group consisting of:
(a) SEQ ID NOs: 21 and 29-33 for Gly m Bd 28 K;
(b) SEQ ID NOs: 22 and 43-44 for Gly m Bd 30 K;
(c) SEQ ID NOs: 23 and 53-54 for Kunitz trypsin inhibitor 1;
(d) SEQ ID NOs: 24 and 62-63 for Kunitz trypsin inhibitor 3;
(e) SEQ ID NOs: 25 and 72-74 for Gly m 8 (2S albumin);
(f) SEQ ID NOs: 26 and 83-90 for Lectin; and
(g) SEQ ID NOs: 27 and 96-104 for lipoxygenase.

17. The system of claim 10, wherein the signature peptides comprise at least one sequence selected from the group consisting of:
(a) SEQ ID NOs: 9 and 34-42 for Gly m Bd 28 K;
(b) SEQ ID NOs: 10 and 45-51 for Gly m Bd 30 K;
(c) SEQ ID NOs: 7 and 55-60 for Kunitz trypsin inhibitor 1;
(d) SEQ ID NOs: 8 and 64-71 for Kunitz trypsin inhibitor 3;
(e) SEQ ID NOs: 11 and 75-81 for Gly m 8 (2S albumin);
(f) SEQ ID NOs: 91-94 for Lectin; and
(e) SEQ ID NOs: 105-120 for lipoxygenase.

18. The system of claim 10, wherein the plant-based sample comprises a soybean seed or part of a soybean seed.

19. A high-throughput method of quantitating at least one allergen with known amino acid sequence and homologous potential allergens in a plant-based sample, comprising using the system of claim 10.


SEQUENCE LISTING (120 sequences)

SEQ ID NO: 1 - protein, 10 aa, Artificial Sequence
  Exemplary signature peptide for Gly m 1
SYPSNATCPR

SEQ ID NO: 2 - protein, 13 aa, Artificial Sequence
  Exemplary signature peptide for Gly m 3
YMVIQGEPGAVIR

SEQ ID NO: 3 - protein, 10 aa, Artificial Sequence
  Exemplary signature peptide for Gly m 5       (beta-conglycinin)
NILEASYDTK

SEQ ID NO: 4 - protein, 7 aa, Artificial Sequence
  Exemplary signature peptide for Gly m 6      (Glycinin) G2
VTAPAMR

SEQ ID NO: 5 - protein, 12 aa, Artificial Sequence
  Exemplary signature peptide for Gly m 6      (Glycinin) G3
NNNPFSFLVPPK

SEQ ID NO: 6 - protein, 7 aa, Artificial Sequence
  Exemplary signature peptide for Gly m 6      (Glycinin) precursor
ADFYNPK

SEQ ID NO: 7 - protein, 11 aa, Artificial Sequence
  Exemplary signature peptide for Kunitz trypsin      inhibitor 1
GGGIEVDSTGK

SEQ ID NO: 8 - protein, 11 aa, Artificial Sequence
  Exemplary signature peptide for Kunitz trypsin      inhibitor 3
GIGTIISSPYR

SEQ ID NO: 9 - protein, 14 aa, Artificial Sequence
  Exemplary signature peptide for Gly m Bd 28 K
NKPQFLAGAASLLR

SEQ ID NO: 10 - protein, 7 aa, Artificial Sequence
  Exemplary signature peptide for Gly m Bd 30 K
GVITQVK

SEQ ID NO: 11 - protein, 12 aa, Artificial Sequence
  Exemplary signature peptide for Gly m 8 (2S      albumin)
IMENQSEELEEK

SEQ ID NO: 12 - protein, 119 aa, Artificial Sequence
  Gly m 1 ABA54898.1 [MW= 12482.64 Da]
MGSKVVASVALLLSINILFISMVSSSSHYDPQPQPSHVTALITRPSCPDLSICLNILGGSLGTVDDCCALIGGLGDIEAIVCLCIQLRALGILNLNRNLQLILNSCGRSYPSNATCPRT

SEQ ID NO: 13 - protein, 131 aa, Artificial Sequence
  Gly m 3 CAA11755.1 [MW= 14100.07 Da]
MSWQAYVDDHLLCGIEGNHLTHAAIIGQDGSVWLQSTDFPQFKPEEITAIMNDFNEPGSLAPTGLYLGGTKYMVIQGEPGAVIRGKKGPGGVTVKKTGAALIIGIYDEPMTPGQCNMVVERLGDYLIDQGY

SEQ ID NO: 14 - protein, 158 aa, Artificial Sequence
  Gly m 4 P26987 [MW= 16771.81 Da]
MGVFTFEDEINSPVAPATLYKALVTDADNVIPKALDSFKSVENVEGNGGPGTIKKITFLEDGETKFVLHKIESIDEANLGYSYSVVGGAALPDTAEKITFDSKLVAGPNGGSAGKLTVKYETKGDAEPNQDELKTGKAKADALFKAIEAYLLAHPDYN

SEQ ID NO: 15 - protein, 605 aa, Artificial Sequence
  Gly m 5 (beta-conglycinin) 121281 [MW= 70293.13      Da]
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERNEEEDEDEEQQRESEESEDSELRRHKNKNPFLFGSNRFETLFKNQYGRIRVLQRFNQRSPQLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 16 - protein, 495 aa, Artificial Sequence
  Gly m 6 Glycinin G1 121276 [MW= 55706.34 Da]
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 17 - protein, 485 aa, Artificial Sequence
  Gly m 6 Glycinin G2 121277 [MW= 54390.76 Da]
MAKLVLSLCFLLFSGCFALREQAQQNECQIQKLNALKPDNRIESEGGFIETWNPNNKPFQCAGVALSRCTLNRNALRRPSYTNGPQEIYIQQGNGIFGMIFPGCPSTYQEPQESQQRGRSQRPQDRHQKVHRFREGDLIAVPTGVAWWMYNNEDTPVVAVSIIDTNSLENQLDQMPRRFYLAGNQEQEFLKYQQQQQGGSQSQKGKQQEEENEGSNILSGFAPEFLKEAFGVNMQIVRNLQGENEEEDSGAIVTVKGGLRVTAPAMRKPQQEEDDDDEEEQPQCVETDKGCQRQSKRSRNGIDETICTMRLRQNIGQNSSPDIYNPQAGSITTATSLDFPALWLLKLSAQYGSLRKNAMFVPHYTLNANSIIYALNGRALVQVVNCNGERVFDGELQEGGVLIVPQNFAVAAKSQSDNFEYVSFKTNDRPSIGNLAGANSLLNALPEEVIQHTFNLKSQQARQVKNNNPFSFLVPPQESQRRAVA

SEQ ID NO: 18 - protein, 481 aa, Artificial Sequence
  Gly m 6 Glycinin G3 121278 [MW= 54241.73 Da]
MAKLVLSLCFLLFSGCCFAFSFREQPQQNECQIQRLNALKPDNRIESEGGFIETWNPNNKPFQCAGVALSRCTLNRNALRRPSYTNAPQEIYIQQGSGIFGMIFPGCPSTFEEPQQKGQSSRPQDRHQKIYHFREGDLIAVPTGFAYWMYNNEDTPVVAVSLIDTNSFQNQLDQMPRRFYLAGNQEQEFLQYQPQKQQGGTQSQKGKRQQEEENEGGSILSGFAPEFLEHAFVVDRQIVRKLQGENEEEEKGAIVTVKGGLSVISPPTEEQQQRPEEEEKPDCDEKDKHCQSQSRNGIDETICTMRLRHNIGQTSSPDIFNPQAGSITTATSLDFPALSWLKLSAQFGSLRKNAMFVPHYNLNANSIIYALNGRALVQVVNCNGERVFDGELQEGQVLIVPQNFAVAARSQSDNFEYVSFKTNDRPSIGNLAGANSLLNALPEEVIQQTFNLRRQQARQVKNNNPFSFLVPPKESQRRVVA

SEQ ID NO: 19 - protein, 562 aa, Artificial Sequence
  Gly m 6 Glycinin G4 121279 [MW= 63587.16 Da]
MGKPFTLSLSSLCLLLLSSACFAISSSKLNECQLNNLNALEPDHRVESEGGLIQTWNSQHPELKCAGVTVSKLTLNRNGLHSPSYSPYPRMIIIAQGKGALGVAIPGCPETFEEPQEQSNRRGSRSQKQQLQDSHQKIRHFNEGDVLVIPPSVPYWTYNTGDEPVVAISLLDTSNFNNQLDQTPRVFYLAGNPDIEYPETMQQQQQQKSHGGRKQGQHQQEEEEEGGSVLSGFSKHFLAQSFNTNEDIAEKLESPDDERKQIVTVEGGLSVISPKWQEQQDEDEDEDEDDEDEQIPSHPPRRPSHGKREQDEDEDEDEDKPRPSRPSQGKRNKTGQDEDEDEDEDQPRKSREWRSKKTQPRRPRQEEPRERGCETRNGVEENICTLKLHENIARPSRADFYNPKAGRISTLNSLTLPALRQFQLSAQYVVLYKNGIYSPHWNLNANSVIYVTRGQGKVRVVNCQGNAVFDGELRRGQLLVVPQNFVVAEQAGEQGFEYIVFKTHHNAVTSYLKDVFRAIPSEVLAHSYNLRQSQVSELKYEGNWGPLVNPESQQGSPRVKVA

SEQ ID NO: 20 - protein, 562 aa, Artificial Sequence
  Gly m 6 Glycinin precursor 75221455 [MW=      63876.47 Da]
MGKPFTLSLSSLCLLLLSSACFAISSSKLNECQLNNLNALEPDHRVEFEGGLIQTWNSQHPELKCAGVTVSKLTLNRNGLHLPSYSPYPRMIIIAQGKGALQCKPGCPETFEEPQEQSNRRGSRSQKQQLQDSHQKIRHFNEGDVLVIPPGVPYWTYNTGDEPVVAISLLDTSNFNNQLDQTPRVFYLAGNPDIEYPETMQQQQQQKSHGGRKQGQHQQEEEEEGGSVLSGFSKHFLAQSFNTNEDIAEKLQSPDDERKQIVTVEGGLSVISPKWQEQQDEDEDEDEDDEDEQIPSHPPRRPSHGKREQDEDEDEDEDKPRPSRPSQGKREQDQDQDEDEDEDEDQPRKSREWRSKKTQPRRPRQEEPRERGCETRNGVEENICTLKLHENIARPSRADFYNPKAGRISTLNSLTLPALRQFQLSAQYVVLYKNGIYSPHWNLNANSVIYVTRGQGKVRVVNCQGNAVFDGELRRGQLLVVPQNFVVAEQAGEQGFEYIVFKTHHNAVTSYLKDVFRAIPSEVLAHSYNLRQSQVSELKYEGNWGPLVNPESQQGSPRVKVA

SEQ ID NO: 21 - protein, 476 aa, Artificial Sequence
  Gly m Bd 28K 12697782 [MW= 52944.36 Da]
MGNKTTLLLLLFVLCHGVATTTMAFHDDEGGDKKSPKSLFLMSNSTRVFKTDAGEMRVLKSHGGRIFYRHMHIGFISMEPKSLFVPQYLDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSRAMKTKIKQGDVFVVPRYFPFCQVASRDGPLEFFGFSTSARKNKPQFLAGAASLLRTLMGPELSAAFGVSEDTLRRAVDAQHEAVILPSAWAAPPENAGKLKMEEEPNAIRSFANDVVMDVF

SEQ ID NO: 22 - protein, 379 aa, Artificial Sequence
  Gly m Bd 30K 84371705 [MW= 42757.81 Da]
MGFLVLLLFSLLGLSSSSSISTHRSILDLDLTKFTTQKQVSSLFQLWKSEHGRVYHNHEEEAKRLEIFKNNLNYIRDMNANRKSPHSHRLGLNKFADITPQEFSKKYLQAPKDVSQQIKMANKKMKKEQYSCDHPPASWDWRKKGVITQVKYQGGCGSGWAFSATGAIEAAHAIATGDLVSLSEQELVDCVEESEGCYNGWHYQSFEWVLEHGGIATDDDYPYRAKEGRCKANKIQDKVTIDGYETLIMSDESTESETEQAFLSAILEQPISVSIDAKDFHLYTGGIYDGENCTSPYGINHFVLLVGYGSADGVDYWIAKNSWGEDWGEDGYIWIQRNTGNLLGVCGMNYFASYPTKEESETLVSARVKGHRRVDHSPL

SEQ ID NO: 23 - protein, 203 aa, Artificial Sequence
  KTI 1 125722 [MW= 22545.94 Da]
MKSTIFFALFLVCAFTISYLPSATAQFVLDTDDDPLQNGGTYYMLPVMRGKGGGIEVDSTGKEICPLTVVQSPNELDKGIGLVFTSPLHALFIAERYPLSIKFGSFAVITLCAGMPTEWAIVEREGLQAVKLAARDTVDGWFNIERVSREYNDYKLVFCPQQAEDNKCEDIGIQIDDDGIRRLVLSKNKPLVVQFQKFRSSTA

SEQ ID NO: 24 - protein, 216 aa, Artificial Sequence
  KTI 3 125020 [ MW= 24005.29 Da]
MKSTIFFLFLFCAFTTSYLPSAIADFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESLAKKNHGLSRSE

SEQ ID NO: 25 - protein, 158 aa, Artificial Sequence
  Gly m 8 (2S albumin) NP_001238443 [MW=       18459.97 Da]
MTKFTILLISLLFCIAHTCSASKWQHQQDSCRKQLQGVNLTPCEKHIMEKIQGRGDDDDDDDDDNHILRTMRGRINYIRRNEGKDEDEEEEGHMQKCCTEMSELRSPKCQCKALQKIMENQSEELEEKQKKKMEKELINLATMCRFGPMIQCDLSSDD

SEQ ID NO: 26 - protein, 280 aa, Artificial Sequence
  Lectin ADC94422 [MW= 30186.22 Da]
MATSNFSIVLSLSLAFFLVLLTKANSTNTVSFTVSKFSPRQQNLIFQGDAAISPSGVLRLTKVDSIDVPTTGSLGRALYATPIQIWDSETGKVASWATSFKFKVFSPNKTADGLAFFLAPVGSKPQSKGGFLGLFNSDSKNKSVQTVAVEFDTYYNAKWDPANRHIGIDVNSIKSVKTASWGLANGQIAQILITYDADTSLLVASLIHPSRKTSYILSETVSLKSNLPEWVNIGFSATTGLNKGFVETHDVFSWSFASKLSDGSTSDTLDLPSFLLNEAI

SEQ ID NO: 27 - protein, 864 aa, Artificial Sequence
  Lipoxygenase CAA39604 [MW= 96817.14 Da]
MFGIFDKGQKIKGTVVLMPKNVLDFNAITSIGKGGVIDTATGILGQGVSLVGGVIDTATSFLGRNISMQLISATQTDGSGNGKVGKEVYLEKHLPTLPTLGARQDAFSIFFEWDASFGIPGAFYIKNFMTDEFFLVSVKLEDIPNHGTIEFVCNSWVYNFRSYKKNRIFFVNDTYLPSATPAPLLKYRKEELEVLRGDGTGKRKDFDRIYDYDVYNDLGNPDGGDPRPILGGSSIYPYPRRVRTGRERTRTDPNSEKPGEVYVPRDENFGHLKSSDFLTYGIKSLSHDVIPLFKSAIFQLRVTSSEFESFEDVRSLYEGGIKLPTDILSQISPLPALKEIFRTDGENVLQFPPPHVAKVSKSGWMTDEEFAREVIAGVNPNVIRRLQEFPPKSTLDPTLYGDQTSTITKEQLEINMGGVTVEEALSTQRLFILDYQDAFIPYLTRINSLPTAKAYATRTILFLKDDGTLKPLAIELSKPHPDGDNLGPESIVVLPATEGVDSTIWLLAKAHVIVNDSGYHQLVSHWLNTHAVMEPFAIATNRHLSVLHPIYKLLYPHYRDTININGLARQSLINADGIIEKSFLPGKYSIEMSSSVYKNWVFTDQALPADLVKRGLAIEDPSAPHGLRLVIEDYPYAVDGLEIWDAIKTWVHEYVSLYYPTDAAVQQDTELQAWWKEAVEKGHGDLKEKPWWPKMQTTEDLIQSCSIIVWTASALHAAVNFGQYPYGGLILNRPTLARRFIPAEGTPEYDEMVKNPQKAYLRTITPKFETLIDLSVIEILSRHASDEIYLGERETPNWTTDKKALEAFKRFGSKLTGIEGKINARNSDPSLRNRTGPVQLPYTLLHRSSEEGLTFKGIPNSISI

SEQ ID NO: 28 - protein, 449 aa, Artificial Sequence
  Gly m Bd 28 K consensus sequence
VLCHGVATTTMAFHDDEGGDKKSPKSLFLMSNSTRVFKTDAGEMRVLKSHGGRIFYRHMHIGFISMEPKSLFVPQYLDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSKAMKTKIKQGDVFVVPRYFPFCQVASRDGPLEFFGFSTSARKNKPQFLAGAASLLRTLMGPELSAAFGVSEDTLRRAVDAQHEAVILPSAWAAPPENAGKLKMEEEP

SEQ ID NO: 29 - protein, 473 aa, Artificial Sequence
  Gly m Bd 28 K Eric BAB21619 473aa
KTTLLLLLFVLCHGVATTTMAFHDDEGGDKKSPKSLFLMSNSTRVFKTDAGEMRVLKSHGGRIFYRHMHIGFISMEPKSLFVPQYLDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSRAMKTKIKQGDVFVVPRYFPFCQVASRDGPLEFFGFSTSARKNKPQFLAGAASLLRTLMGPELSAAFGVSEDTLRRAVDAQHEAVILPSAWAAPPENAGKLKMEEEPNAIRSFANDVVMDVF

SEQ ID NO: 30 - protein, 454 aa, Artificial Sequence
  Gly m Bd 28 K Ping ACD36978.1 455aa
VLCHGVATTTMAFHDDEGGDKKSPKSLFLMSNSTRVFKTDAGEMRVLKSHGGRIFYRHMHIGFISMEPKSLFVPQYLDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGPNGSKAMKTKIKQGDVFVVPRYFPFCQVASRDGPLEFFGFSTSARKNKPQFLAGAASLLRTLMGPELSAAFGVSEDTLRRAVDAQHEAVILPSAWAAPRKMQEAEMEESQMLLKLCQ

SEQ ID NO: 31 - protein, 373 aa, Artificial Sequence
  Gly m Bd 28 K ACD36975.1 373aa
LDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSKAMKTKIKQGDVFVVPRYFPFCQVASRDGPLEFFGFSTSARKNKPQFLAGAASLLRTLMGPELSAAFGVSEDTLRRAVDAQHEAVILPSAWAAPPENAGKLKMEEEP

SEQ ID NO: 32 - protein, 373 aa, Artificial Sequence
  Gly m Bd 28 K Ping ACD36976.1 373aa
LDSNLIIFIRRGEAKLGFIYDDELAERRLKTGDLYMIPSGSAFYLVNIGEGQRLHVICSIDPSTSLGLETFQSFNIGGGANSHSVLSGFEPAILETAFNESRTVVEETFSKELDGPIMFVDDSHAPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSKAMKTKIKQGDVFVVPRYFPFCQVASRDGPLEFFGFSTSARKNKPQFLAGAASLLRTLMGPELSAAFGVSEDTLRRAVDAQHAAVILPSAWAAPPENAGKLKMEEEP

SEQ ID NO: 33 - protein, 320 aa, Artificial Sequence
  Gly m Bd 28 K Ping ACD36974.1 320aa
LDSNLIIFIRRGEAKLGFIYDDELAERRLKTGDLYMIPSGSAFYLVNIGEGQRLHVICSIDPSTSLGLETFQSFYIGGGANSHSVLSGFEPAILETAFNESRTVVEEIFSKELDGPIMFVDDSHVPSLWTKFLQLKKDDKEQQLKKMMQDQEEDEEEKQTSRSWRKLLETVFGKVNEKIENKDTAGSPASYNLYDDKKADFKNAYGWSKALHGGEYPPLSEPDIGVLLVKLSAGSMLAPHVNPISDEYTIVLSGYGELHIGYPNGSKAMKTKIKQGDVFVVPRYFPFCQVASRDGPLEFFGFSTSARKNKPQFLAGAASL

SEQ ID NO: 34 - protein, 12 aa, Artificial Sequence
  Artificial Sequence
LGFIYDDELAER

SEQ ID NO: 35 - protein, 9 aa, Artificial Sequence
  Artificial Sequence
TVVEEIFSK

SEQ ID NO: 36 - protein, 12 aa, Artificial Sequence
  Artificial Sequence
MMQDQEEDEEEK

SEQ ID NO: 37 - protein, 7 aa, Artificial Sequence
  Artificial Sequence
NAYGWSK

SEQ ID NO: 38 - protein, 21 aa, Artificial Sequence
  Artificial Sequence
ALHGGEYPPLSEPDIGVLLVK

SEQ ID NO: 39 - protein, 9 aa, Artificial Sequence
  Artificial Sequence
QGDVFVVPR

SEQ ID NO: 40 - protein, 10 aa, Artificial Sequence
  Artificial Sequence
YFPFCQVASR

SEQ ID NO: 41 - protein, 19 aa, Artificial Sequence
  Artificial Sequence
TLMGPELSAAFGVSEDTLR

SEQ ID NO: 42 - protein, 11 aa, Artificial Sequence
  Artificial Sequence
SFANDVVMDVF

SEQ ID NO: 43 - protein, 379 aa, Artificial Sequence
  Gly m Bd 30 K Ping AAB09252.1 379aa (also      serves as consensus sequence)
MGFLVLLLFSLLGLSSSSSISTHRSILDLDLTKFTTQKQVSSLFQLWKSEHGRVYHNHEEEAKRLEIFKNNSNYIRDMNANRKSPHSHRLGLNKFADITPQEFSKKYLQAPKDVSQQIKMANKKMKKEQYSCDHPPASWDWRKKGVITQVKYQGGCGRGWAFSATGAIEAAHAIATGDLVSLSEQELVDCVEESEGSYNGWQYQSFEWVLEHGGIATDDDYPYRAKEGRCKANKIQDKVTIDGYETLIMSDESTESETEQAFLSAILEQPISVSIDAKDFHLYTGGIYDGENCTSPYGINHFVLLVGYGSADGVDYWIAKNSWGEDWGEDGYIWIQRNTGNLLGVCGMNYFASYPTKEESETLVSARVKGHRRVDHSPL

SEQ ID NO: 44 - protein, 379 aa, Artificial Sequence
  Gly m Bd 30 K Ping P22895.1 379aa
MGFLVLLLFSLLGLSSSSSISTHRSILDLDLTKFTTQKQVSSLFQLWKSEHGRVYHNHEEEAKRLEIFKNNSNYIRDMNANRKSPHSHRLGLNKFADITPQEFSKKYLQAPKDVSQQIKMANKKMKKEQYSCDHPPASWDWRKKGVITQVKYQGGCGRGWAFSATGAIEAAHAIATGDLVSLSEQELVDCVEESEGSYNGWQYQSFEWVLEHGGIATDDDYPYRAKEGRCKANKIQDKVTIDGYETLIMSDESTESETEQAFLSAILEQPISVSIDAKDFHLYTGGIYDGENCTSPYGINHFVLLVGYGSADGVDYWIAKNSWGFDWGEDGYIWIQRNTGNLLGVCGMNYFASYPTKEESETLVSARVKGHRRVDHSPL

SEQ ID NO: 45 - protein, 9 aa, Artificial Sequence
  Artificial Sequence
SILDLDLTK

SEQ ID NO: 46 - protein, 5 aa, Artificial Sequence
  Artificial Sequence
FTTQK

SEQ ID NO: 47 - protein, 7 aa, Artificial Sequence
  Artificial Sequence
NNLNYIR

SEQ ID NO: 48 - protein, 11 aa, Artificial Sequence
  Artificial Sequence
FADITPQEFSK

SEQ ID NO: 49 - protein, 15 aa, Artificial Sequence
  Artificial Sequence
EQYSCDHPPASWDWR

SEQ ID NO: 50 - protein, 40 aa, Artificial Sequence
  Artificial Sequence
VTIDGYETLIMSDESTESETEQAFLSAILEQPISVSIDAK

SEQ ID NO: 51 - protein, 20 aa, Artificial Sequence
  Artificial Sequence
NTGNLLGVCGMNYFASYPTK

SEQ ID NO: 52 - protein, 203 aa, Artificial Sequence
  KTI 1 consensus sequence
MKSTIFFALFLVCAFTISYLPSATAQFVLDTDDDPLQNGGTYYMLPVMRGKGGGIEGASTGKEICPLTVVQSPNELDKGIGLVFSSPLHALFIAERYPLSIKFGSFAVISLCGGMPTKWAIVEREGLQAVTLAARDTVDGWFNIERVSREYNDYKLVFCPQNAEDNKCEDIGIQIDNDGIRRLVLSKNKPLVVQFQKFRSSTA

SEQ ID NO: 53 - protein, 204 aa, Artificial Sequence
  KTI 1 AAB23483.1 204aa
MKSTIFFALFLVCAFTISYLPSATAQFVLDTDDDPLQNGGTYYMLPVMRGKSGGIEGNSTGKEICPLTVVQSPNKHNKGIGLVFKSPLHALFIAERYPLSIKFDSFAVIPLCGVMPTKWAIVEREGLQAVTLAARDTVDGWFNIERVSREYNDYYKLVFCPQEAEDNKCEDIGIQIDNDGIRRLVLSKNKPLVVEFQKFRSSTA

SEQ ID NO: 54 - protein, 208 aa, Artificial Sequence
  KTI 1 CAA56343.1 208aa
MKSTTSLALFLLCALTSSYQPSATADIVFDTEGNPIRNGGTYYVLPVIRGKGGGIEFAKTETETCPLTVVQSPFEGLQRGLPLIISSPFKILDITEGLILSLKFHLCTPLSLNSFSVDRYSQGSARRTPCQTHWLQKHNRCWFRIQRASSESNYYKLVFCTSNDDSSCGDIVAPIDREGNRPLIVTHDQNHPLLVQFQKVEAYESSTA

SEQ ID NO: 55 - protein, 16 aa, Artificial Sequence
  Artificial Sequence
EICPLTVVQSPNELDK

SEQ ID NO: 56 - protein, 7 aa, Artificial Sequence
  Artificial Sequence
EGLQAVK

SEQ ID NO: 57 - protein, 12 aa, Artificial Sequence
  Artificial Sequence
LVFCPQQAEDNK

SEQ ID NO: 58 - protein, 14 aa, Artificial Sequence
  Artificial Sequence
CEDIGIQIDDDGIR

SEQ ID NO: 59 - protein, 5 aa, Artificial Sequence
  Artificial Sequence
LVLSK

SEQ ID NO: 60 - protein, 10 aa, Artificial Sequence
  Artificial Sequence
NKPLVVQFQK

SEQ ID NO: 61 - protein, 217 aa, Artificial Sequence
  KTI 3 consensus sequence
MKSTIFFALFLFCAFTTSYLPSAIADFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESLAKKNHGLSRSE

SEQ ID NO: 62 - protein, 217 aa, Artificial Sequence; identical to SEQ ID NO: 61
  KTI 3 CAA45777.1 217aa

SEQ ID NO: 63 - protein, 217 aa, Artificial Sequence
  KTI 3 CAA45778.1 217aa
MKSTIFFALFLFCAFTTSYLPSAIADFVLDNEGNPLDSGGTYYILSDITAFGGIRAAPTGNERCPLTVVQSRNELDKGIGTIISSPFRIRFIAEGNPLRLKFDSFAVIMLCVGIPTEWSVVEDLPEGPAVKIGENKDAVDGWFRIERVSDDEFNNYKLVFCTQQAEDDKCGDIGISIDHDDGTRRLVVSKNKPLVVQFQKVDKESLAKKNHGLSRSE

SEQ ID NO: 64 - protein, 9 aa, Artificial Sequence
  Artificial Sequence
CPLTVVQSR

SEQ ID NO: 65 - protein, 5 aa, Artificial Sequence
  Artificial Sequence
NELDK

SEQ ID NO: 66 - protein, 5 aa, Artificial Sequence
  Artificial Sequence
IGENK

SEQ ID NO: 67 - protein, 8 aa, Artificial Sequence
  Artificial Sequence
DAMDGWFR

SEQ ID NO: 68 - protein, 12 aa, Artificial Sequence
  Artificial Sequence
LVFCPQQAEDDK

SEQ ID NO: 69 - protein, 15 aa, Artificial Sequence
  Artificial Sequence
CGDIGISIDHDDGTR

SEQ ID NO: 70 - protein, 5 aa, Artificial Sequence
  Artificial Sequence
LVVSK

SEQ ID NO: 71 - protein, 10 aa, Artificial Sequence
  Artificial Sequence
NKPLVVQFQK

SEQ ID NO: 72 - protein, 173 aa, Artificial Sequence
  2S albumin Glyma13g36400.1 BLAST 174aa
MPPPSLHFTSPINSKMTKFTILLISLLFCIAHTCSASKWQHQQDSCRKQLQGVNLTPCEKHIMEKIQGRGDDDDDDDDDNHILRTMRGRINYIRRNEGKDEDEEEEGHMQKCCTEMSELRSPKCQCKALQKIMENQSEELEEKQKKKMEKELINLATMCRFGPMIQCDLSSDD

SEQ ID NO: 73 - protein, 158 aa, Artificial Sequence
  2S albumin NP_001234950 BLAST 158aa
MTKFTILLISLLFCIAHTCSASEWQHQQDSCRKQLQGVNLTPCEKHIMEKIQGRGDDDDDDDDDNHILRTMRGRINYIRRNEGKDEDEEEEGHMQKCRTEMSELRSPKCQCKALQKIMENQSEELEEKQKKKMEKELINLATMCRFGPMIQCDLSSDD

SEQ ID NO: 74 - protein, 155 aa, Artificial Sequence
  2S albumin Glyma12g34160.1 BLAST 156aa
MTKLTILLIALLFIAHTCCASKWQQHQQESCREQLKGINLNPCEHIMEKIQAGRRGEDGSDEDHILIRTMPGRINYIRKKEGKEEEEEGHMQKCCSEMSELKSPICQCKALQKIMDNQSEQLEGKEKKQMERELMNLAIRCRLGPMIGCDLSSDD

SEQ ID NO: 75 - protein, 9 aa, Artificial Sequence
  Artificial Sequence
WQHQQDSCR

SEQ ID NO: 76 - protein, 12 aa, Artificial Sequence
  Artificial Sequence
QLQGVNLTPCEK

SEQ ID NO: 77 - protein, 5 aa, Artificial Sequence
  Artificial Sequence
HIMEK

SEQ ID NO: 78 - protein, 12 aa, Artificial Sequence
  Artificial Sequence
DEDEEEEGHMQK

SEQ ID NO: 79 - protein, 9 aa, Artificial Sequence
  Artificial Sequence
CCTEMSELR

SEQ ID NO: 80 - protein, 10 aa, Artificial Sequence
  Artificial Sequence
ELINLATMCR

SEQ ID NO: 81 - protein, 13 aa, Artificial Sequence
  Artificial Sequence
FGPMIQCDLSSDD

SEQ ID NO: 82 - protein, 280 aa, Artificial Sequence
  Lectin consensus sequence
MATSNFSIVLSLSLAFFLVLLTKANSTNTVSFTFSKFSPRQPNLILQGDAAISSSGVLRLTKVDSNGVPTSGSLGRALYAAPIQIWDSETGKVASWATSFKFNVFAPNKTADGLAFFLAPVGSKPQSKGGFLGLFNSDSKDKSLQTVAVEFDTYSNKKWDPANRHIGIDVNSIKSVKTASWGLANGQVAQILITYDAATSLLVASLIHPSRKTSYILSETVSLKSNLPEWVSIGFSATTGLNEGSVETHDVISWSFASKLSDGSTSDALDLPSFLLNEAI

SEQ ID NO: 83 - protein, 280 aa, Artificial Sequence
  Lectin XP_003535884 BLAST 280aa
MATSNFSIVLSLSLALFLMLLTKANSTNTVSFTTSKFSPRQQNLILQGDAAISPSGVLRLTKVDSYGVPTSRSLGRALYAAPIQIWDSETGKVASWATSFKFNVFSPDKTADGLAFFLAPVGSKPQYKAGFLGLFNSDSKNMSLQTVAVEFDTYYNQKWDPASRHIGIDVNSIKSVKTAPWGFANGQVAQILITYNADTSLLVASLVHPSRKTSYILSETVSLKSNLPEWVNVGFSATTGANKGFAETHDVFSWSFASKLSDGSTSDTLDLASFLLNEAI

SEQ ID NO: 84 - protein, 270 aa, Artificial Sequence
  Lectin Glyma10g15480.1 BLAST
MATSNFSIVLSLSLALFLMLLTKANSTNTVSFTTSKFSPRQQNLILQGDAAISPSGVLRLTKVDSYGVPTSRSLGRALYAAPIQIWDSETGKVASWATSFKFNVFSPDKTADGLAFFLAPVGSKPQYKAGFLGLFNSDSKNMSLQTVAWDPASRHIGIDVNSIKSVKTAPWGFANGQVAQILITYNADTSLLVASLVHPSRKTSYILSETVSLKSNLPEWVNVGFSATTGANKGFAETHDVFSWSFASKLSDGSTSDTLDLASFLLNEAI

SEQ ID NO: 85 - protein, 282 aa, Artificial Sequence
  Lectin NP_001237210 BLAST 282aa
MATSNFSIVLSVSLAFFLVLLTKAHSTDTVSFTFNKFNPVQPNIMLQKDASISSSGVLQLTKVGSNGVPTSGSLGRALYAAPIQIWDSETGKVASWATSFKFNIFAPNKSNSADGLAFFLAPVGSQPQSDDGFLGLFNSPLKDKSLQTVAIEFDTFSNKKWDPANRHIGIDVNSIKSVKTASWGLSNGQVAEILVTYNAATSLLVASLIHPSKKTSYILSDTVNLKSNLPEWVSVGFSATTGLHEGSVETHDVISWSFASKLSDGSSNDALDLPSFVLNEAI

SEQ ID NO: 86 - protein, 297 aa, Artificial Sequence
  Lectin Glyma02g18090.1 BLAST
MKVLCIIFEFKQIKAMATSNFSIVLSVSLAFFLVLLTKAHSTDTVSFTFNKFNPVQPNIMLQKDASISSSGVLQLTKVGSNGVPTSGSLGRALYAAPIQIWDSETGKVASWATSFKFNIFAPNKSNSADGLAFFLAPVGSQPQSDDGFLGLFNSPLKDKSLQTVAIEFDTFSNKKWDPANRHIGIDVNSIKSVKTASWGLSNGQVAEILVTYNAATSLLVASLIHPSKKTSYILSDTVNLKSNLPEWVSVGFSATTGLHEGSVETHDVISWSFASKLSDGSSNDALDLPSFVLNEAI

SEQ ID NO: 87 - protein, 282 aa, Artificial Sequence
  Lectin ACU23599 BLAST 282aa
MATSNFSIVLSVSLAFFLVLLTKAHPTDTVSFTFNKFNPVQPNIMLQKDASISSSGVLQLTKVGSNGVPTSGSLGRALYAAPIQIWDSETGKVASWATSFKFNIFAPNKSNSADGLAFFLAPVGSQPQSDDGFLGLFNSPLKDKSLQTVAIEFDTFSNKKWDPANRHIGIDVDSIKSIKTASWGLSNGQVAEILVTYNAATSLLVASLIHPSKKTSYILSDTVNLKSNLPEWVSVGFSATTGLHEGSVETHDVISWSFASKLSDGSSNDALDLPSFVLNEAI

SEQ ID NO: 88 - protein, 280 aa, Artificial Sequence
  Lectin CAH60173 BLAST 280aa
MASSKFSTVISFSLALFLVLLTQANSTNIFSFNFQTFDSPNLIFQGDASVSSSGQLRLTKVKGNGKPTAASLGRAFYSAPIQIWDSTTGNVASFATSFTFNILAPNKSNSADGLAFALVPVGSQPKSNGGFLGLFDNATYDSSAQTVAVEFDTYSNPKWDPENRHIGIDVNSIESIRTASWGLANGQNAEILITYDSSTKLLVASLVHPSRRTSYIVSERVDLKSVLPEWVSIGFSATTGLLEGSIETHDVLSWSFASKLSDDTTSEGLNLANFVLNKIL

SEQ ID NO: 89 - protein, 228 aa, Artificial Sequence
  Lectin Glyma10g01620.2 BLAST 228aa
MATSKFHTQKPLFVVLSVVVVLLTMTKVNSTKPFLSPGTSSCRTNRTLILQGDALVTSSRKSLGRALYSTPIHIWDSEIGSVASFAASFNFTVYASDIANLADGLAFFLAPIDTQPQTRGGYLGLYNNPSNSSWGLANDQVTNVLITYDASTNLLVASLVHPSQRSSYILSDVLDLKVALPEWVRIGFSATTGLNVASETHDVHSWSFSSNLPFGSSNTNPSDFAIFI

SEQ ID NO: 90 - protein, 285 aa, Artificial Sequence
  Lectin Glyma02g01590.1 BLAST 285aa
MATSKLKTQNVVVSLSLTLTLVLVLLTSKANSAETVSFSWNKFVPKQPNMILQGDAIVTSSGKLQLNKVDENGTPKPSSLGRALYSTPIHIWDKETGSVASFAASFNFTFYAPDTKRLADGLAFFLAPIDTKPQTHAGYLGLFNENESGDQVVAVEFDTFRNSWDPPNPHIGINVNSIRSIKTTSWDLANNKVAKVLITYDASTSLLVASLVYPSQRTSNILSDVVDLKTSLPEWVRIGFSAATGLDIPGESHDVLSWSFASNLPHASSNIDPLDLTSFVLHEAI

SEQ ID NO: 91 - protein, 6 aa, Artificial Sequence
  Artificial Sequence
VFSPNK

SEQ ID NO: 92 - protein, 13 aa, Artificial Sequence
  Artificial Sequence
ANSTNTVSFTVSK

SEQ ID NO: 93 - protein, 19 aa, Artificial Sequence
  Artificial Sequence
QQNLIFQGDAAISPSGVLR

SEQ ID NO: 94 - protein, 19 aa, Artificial Sequence
  Artificial Sequence
TADGLAFFLAPVGSKPQSK

SEQ ID NO: 95 - protein, 864 aa, Artificial Sequence
  Lipoxygenase consensus sequence
MGGIFDKGQKIKGTVVLMPKNVLDFNAITSIGKGGVIDTATGILGAGVSLVGGVIDTATAFLGRNISMQLISATQTDGSGNGKVGKEVYLEKHLPTLPTLGARQDAFSIFFEWDASFGIPGAFYIKNFMTDEFFLVSVKLEDIPNHGTIEFVCNSWVYNFKSYKKNRIFFVNDTYLPSATPAPLLKYRKEELEVLRGDGTGKRKDFDRIYDYDVYNDLGNPDGGDPRPILGGSSIYPYPRRVRTGRERTRTDPNSEKPGEVYVPRDENFGHLKSSDFLTYGIKSLSHDVIPLFKSAIFQLRVTSSEFDSFEDVRSLYEGGIKLPTDILSQISPLPALKEIFRTDGENVLQFPPPHVAKVSKSGWMTDEEFAREMIAGVNPNVIRRLQEFPPKSTLDPTLYGDQTSTITKEQLEINMGGVTVEEALSTQRLFILDYQDAFIPYLTRINSLPTAKAYATRTILFLKDDGTLKPLAIELSKPHPDGDNLGPESIVVLPATEGVDSTIWLLAKAHVIVNDSGYHQLVSHWLNTHAVMEPFAIATNRHLSVLHPIYKLLYPHYRDTININGLARQSLINADGIIEKSFLPGKYSIEMSSSVYKNWVFTDQALPADLVKRGLAIEDPSAPHGLRLVIEDYPYAVDGLEIWDAIKTWVHEYVSLYYPTDAAVQQDTELQAWWKEAVEKGHGDLKDKPWWPKMQTTEDLIQSCSIIIWTASALHAAVNFGQYPYGGLILNRPTLARRFIPEEGTPEYDEMVKNPQKAYLRTITPKFETLIDLSVIEILSRHASDEIYLGERDTPNWTTDKKALEAFKKFGSKLTGIEGKINARNSDPSLRNRTGPVQLPYTLLHRSSEEGLTFKGIPNSISI

SEQ ID NO: 96 - protein, 864 aa, Artificial Sequence
  Lipoxygenase 2IUK_A BLAST 864aa
MFGIFDKGQKIKGTVVLMPKNVLDFNAITSIGKGGVIDTATGILGQGVSLVGGVIDTATSFLGRNISMQLISATQTDGSGNGKVGKEVYLEKHLPTLPTLGARQDAFSIFFEWDASFGIPGAFYIKNFMTDEFFLVSVKLEDIPNHGTIEFVCNSWVYNFRSYKKNRIFFVNDTYLPSATPAPLLKYRKEEFEVLRGDGTGKRKDFDRIYDYDVYNDLGNPDGGDPRPILGGCSIYPYPLRVRTGRERTRTDPNSEKPGEVYVPRDENFGHLKSSDFLTYGIKSLSHDVIPLFKSAIFQLRVTSSEFESFEDVRSLYEGGIKLPTDILSQISPLPALKEIFRTDGENVLQFPPPHVAKVSKSGVMTDEEFAREVIAGVNPNVIRRLQEFPPKSTLDPTLYGDQTSTITKEQLEINMGGVTVEEALSTQRLFILDYQDAFIPYLTRINSLPTAKAYATRTILFLKDDGTLKPLAIELSKPHPDGDNLGPESIVVLPATEGVDSTIWLLAKAHVIVNDSGYHQLVSHWLNTHAVMEPFAIATNRHLSVLHPIYKLLYPHYRDTININGLARQSLINADGIIEKSFLPGKYSIEMSSSVYKNWVFTHQALPADLVKRGLAIEDPSAPHGLRLVIEDYPYAVDGLEIWDAIKTWVHEYVSLYYPTDAAVQQDTELQAWWKEAVEKGHGDLKEKPWWPKKQTTEDLIQSCSIIVWTASALHAAVNFGQYPYGGLILNRPTLARRFIPAEGTPEYDEMVKNPQKAYLRTITPKFETLIDLSVIEILSRHASDEIYLGERETPNWTTDKKALEAFKRFGSKLTGIEGKINARNSDPSLRNRTGPVQLPYTLLHRSSEEGLTFKGIPNSISI

SEQ ID NO: 97 - protein, 864 aa, Artificial Sequence
  Lipoxygenase NP_001238676 BLAST 864aa
MFGIFDKGQKIKGTVVLMPKNVLDFNAITSIGKGGVIDTATGILGQGVSLVGGVIDTATSFLGRNISMQLISATQTDGSGNGKVGKEVYLEKHLPTLPTLGARQDAFSIFFEWDASFGIPGAFYIKNFMTDEFFLVSVKLEDIPNHGTIEFVCNSWVYNFRSYKKNRIFFVNDTYLPSATPAPLLKYRKEELEVLRGDGTGKRKDFDRIYDYDVYNDLGNPDGGDPRPILGGCSIYPYPLRVRTGRERTRTDPNSEKPGEVYVPRDENFGHLKSSDFLTYGIKSLSHDVIPLFKSAIFQLRVTSSEFESFEDVRSLYEGGIKLPTDILSQISPLPALKEIFRTDGENVLQFPPPHVAKVSKSGWMTDEEFAREVIAGVNPNVIRRLQEFPPKSTLDPTLYGDQTSTITKEQLEINMGGVTVEEALSTQRLFILDYQDAFIPYLTRINSLPTAKAYATRTILFLKDDGTLKPLAIELSKPHPDGDNLGPESIVVLPATEGVDSTIWLLAKAHVIVNDSGYHQLVSHWLNTHAVMEPFAIATNRHLSVLHPIYKLLYPHYRDTININGLARQSLINADGIIEKSFLPGKYSIEMSSSVYKNWVFTHQALPADLVKRGLAIEDPSAPHGLRLVIEDYPYAVDGLEIWDAIKTWVHEYVSLYYPTDAAVQQDTELQAWWKEAVEKGHGDLKEKPWWPKKQTTEDLIQSCSIIVWTASALHAAVNFGQYPYGGLILNRPTLARRFIPAEGTPEYDEMVKNPQKAYLRTITPKFETLIDLSVIEILSRHASDEIYLGERETPNWTTDKKALEAFKRFGSKLTGIEGKINARNSDPSLRNRTGPVQLPYTLLHRSSEEGLTFKGIPNSISI

SEQ ID NO: 98 - protein, 867 aa, Artificial Sequence
  Lipoxygenase Glyma08g20220.1 BLAST 867aa
MLGLFDKSHKIKGTVVLMPKSVLDINDLNSVKNGGVGGVVSGIFGAVADVTGQIVDTATAIFSRNVSFKLISATSTDAKGNGKVGNETFLEKHLPTLPTLGDRRDAYDIHFEWDANFGIPGAFYIRNYTYDEFFLVSVTLEDIPNHGTIHFVCNSWVYNFKDYDKKDRIFFANKTYLPSATPGPLVKYREEELKILRGDGTGERKEHERIYDYDVYNDLGNPDEDVKLARPVLGGSSTYPYPRRVRTGRKATKKDPKSERPASELYMPRDEKFGHLKSSDFLTYGIKSLSQKLLPSLENVFDSDLTWNEFDSFEEVRDLYEGGIKVPTGVLSDISPIPIFKEIFRTDGESVLQFPPPHVVQVTKSAWMTDDEFAREMIAGVNPNVIRLLKEFPPQSKLDPSLYGDQSSTITKEHLEINMDGVTVEEALNGQRLFILDYQDAFMPYLTRINALPSAKAYATRTILLLKDDGTLKPLAIELSKPHPSGDNLGAESKVVLPADQGVESTIWLLAKAHVIVNDSGYHQLMSHWLNTHAVTEPFIIATNRRLSVLHPIYKLLYPHYRDTININGLARNALINAGGVIEESFLPGRYSIEMSSAVYKNWVFTDQALPVDLIKRGMAVEDPSSPHGLRLAVEDYPYAVDGLEIWDAIKSWVQEYVSLYYPTDLAIQQDTELQAWWKEVVEKGHGDLKDKPWWPKMQTRQELIQSCSTIIWIASALHAAVNFGQYPYGGFILNRPTLSRRWIPEPGTKEYDEMVESPQTAYLRTITPKRQTIIDLTVIEILSRHASDEIYLGERDNPNWTSDSKALEAFKKFGSKLAEIEGKITARNKDSNKKNRYGPVQLPYTLLLPTSEEGLTFRGIPNSISI

SEQ ID NO: 99 - protein, 864 aa, Artificial Sequence; identical to SEQ ID NO: 27
  Lipoxygenase P24095 BLAST 864aa

SEQ ID NO: 100 - protein, 866 aa, Artificial Sequence
  Lipoxygenase Glyma07g00900.1 BLAST 866aa
MTGGMFGRKGQKIKGTVVLMPKNVLDFNAITSVGKGSAKDTATDFLGKGLDALGHAVDALTAFAGHSISLQLISATQTDGSGKGKVGNEAYLEKHLPTLPTLGARQEAFDINFEWDASFGIPGAFYIKNFMTDEFFLVSVKLEDIPNHGTINFVCNSWVYNFKSYKKNRIFFVNDTYLPSATPAPLLKYRKEELEVLRGDGTGKRKDFDRIYDYDVYNDLGNPDGGDPRPILGGSSIYPYPRRVRTGRERTRTDPNSEKPGEVYVPRDENFGHLKSSDFLTYGIKSLSHDVIPLFKSAIFQLRVTSSEFESFEDVRSLYEGGIKLPTDILSQISPLPALKEIFRTDGENVLQFPPPHVAKVSKSGWMTDEEFAREVIAGVNPNVIRRLQEFPPKSTLDPTLYGDQTSTITKEQLEINMGGVTVEEALSTQRLFILDYQDAFIPYLTRINSLPTAKAYATRTILFLKDDGTLKPLAIELSKPHPDGDNLGPESIVVLPATEGVDSTIWLLAKAHVIVNDSGYHQLVSHWLNTHAVMEPFAIATNRHLSVLHPIYKLLYPHYRDTININGLARQSLINADGIIEKSFLPGKYSIEMSSSVYKNWVFTDQALPADLVKRGLAIEDPSAPHGLRLVIEDYPYAVDGLEIWDAIKTWVHEYVSLYYPTDAAVQQDTELQAWWKEAVEKGHGDLKEKPWWPKMQTTEDLIQSCSIIVWTASALHAAVNFGQYPYGGLILNRPTLARRFIPAEGTPEYDEMVKNPQKAYLRTITPKFETLIDLSVIEILSRHASDEIYLGERETPNWTTDKKALEAFKRFGSKLTGIEGKINARNSDPSLRNRTGPVQLPYTLLHRSSEEGLTFKGIPNSISI

SEQ ID NO: 101 - protein, 859 aa, Artificial Sequence
  Lipoxygenase NP_001235189 BLAST 859aa
MTGGMFGRKGQKIKGTVVLMPKNVLDFNAITSVGKGSAKDTATDFLGKGLDALGHAVDALTAFAGHSISLQLISATQTDGSGKGKVGNEAYLEKHLPTLPTLGARQEAFDINFEWDASFGIPGAFYIKNFMTDEFFLVSVKLEDIPNHGTINFVCNSWVYNFKSYKKNRIFFVNDTYLPSATPGPLVKYRQEELEVLRGDGTGKRRDFDRIYDYDIYNDLGNPDGGDPRPIIGGSSNYPYPRRVRTGREKTRKDPNSEKPGEIYVPRDENFGHLKSSDFLTYGIKSLSQNVIPLFKSIILNLRVTSSEFDSFDEVRGLFEGGIKLPTNILSQISPLPVLKEIFRTDGENTLQFPPPHVIRVSKSGWMTDDEFAREMIAGVNPNVIRRLQEFPPKSTLDPATYGDQTSTITKQQLEINLGGVTVEEAISAHRLFILDYHDAFFPYLTKINSLPIAKAYATRTILFLKDDGSLKPLAIELSKPATVSKVVLPATEGVESTIWLLAKAHVIVNDSGYHQLISHWLNTHAVMEPFAIATNRHLSVLHPIYKLLYPHYKDTININGLARQSLINAGGIIEQTFLPGKYSIEMSSVVYKNWVFTDQALPADLVKRGLAVEDPSAPHGLRLVIEDYPYAVDGLEIWDAIKTWVHEYVSVYYPTNAAIQQDTELQAWWKEVVEKGHGDLKDKPWWPKLQTVEDLIQSCSIIIWTASALHAAVNFGQYPYGGYIVNRPTLARRFIPEEGTKEYDEMVKDPQKAYLRTITPKFETLIDISVIEILSRHASDEVYLGQRDNPNWTTDSKALEAFKKFGNKLAEIEGKITQRNNDPSLKSRHGPVQLPYTLLHRSSEEGMSFKGIPNSISI

SEQ ID NO: 102 - protein, 865 aa, Artificial Sequence
  Lipoxygenase Glyma07g03910.1 BLAST 865aa
MFGILGGNKGHKIKGTVVLMSKNVLDFNEIVSTTQGGLVGAATGIFGAATGIVGGVVDGATAIFSRNIAIQLISATKTDGLGNGKVGKQTYLEKHLPSLPTLGDRQDAFSVYFEWDNDFGIPGAFYIKNFMQSEFFLVSVTLEDIPNHGTIHFVCNSWVYNAKSYKRDRIFFANKTYLPNETPTPLVKYRKEELENLRGDGKGERKEYDRIYDYDVYNDLGNPDKSNDLARPVLGGSSAYPYPRRGRTGRKPTTKDSKSESPSSSTYIPRDENFGHLKSSDFLTYGIKSIAQTVLPTFQSAFGLNAEFDRFDDVRGLFEGGIHLPTDALSKISPLPVLKEIFRTDGEQVLKFPPPHVIKVSKSAWMTDEEFGREMLAGVNPCLIECLQVFPPKSKLDPTVYGDQTSTITKEHLEINLGGLSVEQALSGNRLFILDHHDAFIAYLRKINDLPTAKSYATRTILFLKDDGTLKPLAIELSLPHPRGDEFGAVSRVVLPADQGAESTIWLIAKAYVVVNDSCYHQLMSHWLNTHAVIEPFVIATNRHLSVLHPIYKLLLPHYRDTMNINGLARQSLINAGGIIEQSFLPGPFAVEMSSAVYKGWVFTDQALPADLIKRGMAVEDPSSPYGLRLVIDDYPYAVDGLEIWSAIQTWVKDYVSLYYATDDAVKKDSELQAWWKEAVEKGHGDLKDKPWWPKLNTLQDLIHICCIIIWTASALHAAVNFGQYPYGGFILNRPTLTRRLLPEPGTKEYGELTSNHQKAYLRTITGKTEALVDLTVIEILSRHASDEVYLGQRDNPNWTDDTKAIQAFKKFGNKLKEIEDKISGRNKNSSLRNRNGPAQMPYTVLLPTSGEGLTFRGIPNSISI

SEQ ID NO: 103 - protein, 868 aa, Artificial Sequence
  Lipoxygenase Glyma07g03920.2 BLAST 868aa
MLIGSLLNRRPKIKGTVVLMTKNVFDVNDFMATTRGGPAAVAGGIFGAAQDIVGGIVDGATAIFSRNIAIQLISATKSENALGHGKVGKLTYLEKHLPSLPNLGDRQDAFDVYFEWDESFGIPGAFYIKNYMQSEFFLVSFKLEDVPNHGTILFACNSWVYNAKLYKKDRIFFANKAYLPNDTPTPLVKYRKEELENLRGDGRGERKELDRIYDYDVYNDLGNPDENDDLARPILGGSSKHPYPRRGRTGRKPTKKDPRCERPTSDTYIPRDENFGHLKSSDFLTYAIKSLTQNVLPQFNTAFGFNNEFDSFEDVRCLFDGGVYLPTDVLSKISPIPVLKEIFRTDGEQALKFPPPHVIKVRESEWMTDEEFGREMLAGVNPGMIQRLQEFPPKSKLDPTEFGDQTSTITKEHLEINLGGLTVEQALKGNKLFILDHHDAFIPFMNLINGLPTAKSYATRTILFLQDDGTLKPLAIELSLPHPRGHEFGADSRVVLPPAAVNSAEGTIWLIAKAYVAVNDTGYHQLISHWLNTHATIEPFVIATNRHLSVLHPIHKLLLPHYRDTMNINALARQSLINADGVIERSFLPGKYSLEMSSAVYKSWVFTDQALPADLIKRGMAIEDPCAPHGLRLVIEDYPYAVDGLEIWDAIQTWVKNYVSLYYPTDDAIKKDSELQAWWKEAVETGHGDLKDKPWWPKLNTPQDLVHICSIIIWIASALHAAVNFGQYPYGGLILNRPTLTRRFLPEPGSKEYEELSTNYQKAYLRTITRKIEALVDLSVIEILSRHASDEIYLGKRDSDDWTDDQKAIQAFEKFGTKLKEIEAKINSRNKDSSLRNRNGPVQMPYTVLLPTSEEGLTFRGIPNSISI

SEQ ID NO: 104 - protein, 860 aa, Artificial Sequence
  Lipoxygenase Glyma08g20190.1 BLAST 860aa
MYSGVKGLFNRSQKVKGTVVLMRKNVLDINSITSVRGLIGTGINIIGSTIDGLTSFLGRSVCLQLISATKADGNGNGVVGKKTYLEGIITSIPTLGAGQSAFTIHFEWDADMGIPGAFLIKNYMQVELFLVSLTLEDIPNQGSMHFVCNSWVYNSKVYEKDRIFFASETYVPSETPGPLVTYREAELQALRGNGTGKRKEWDRVYDYDVYNDLGNPDSGENFARPVLGGSLTHPYPRRGRTGRKPTKKDPNSEKPGEAYIPRDENFGHLKSSDFLTYGLKSLTRSFLPALKTVFDINFTPNEFDSFEEVRALCEGGIKLPTDILSKISPLPVLKEIFRTDGESVLKFSVPDLIKVSKSAWMTDEEFAREMIAGVNPCVIRRLQEFPPQSKLDPSVYGDQTSKMTIDHLEINLEGLTVDKAIKDQRLFILDHHDTFMPFLRRIDESKSSKAYATRTILFLKDDGTLKPLAIELSLPHPGQQQLGAYSKVILPANQGVESTIWLLAKAHVIVNDSCYHQLISHWLNTHAVIEPFVIATNRNLSILHPIYKLLFPHYRDTMNINALARQSLINADGFIEKTFLGGKYAVEISSSGYKNWVFLDQALPADLIKRGMAIEDSSCPNGLRLVIEDYPYAVDGLEIWDAIKTWVQEYVSLYYATNDAIKKDHELQAWWKEVVEKGHGDLKDKPWWPKMQTLQELIQSCSTIIWIASALHAAVNFGQYPYGGFILNRPTLSRRWIPEEGTPEYDEMTKNPQKAYLRTITPKFQALVDLSVIEILSRHASDEVYLGQRDNPNWTSNPKAIEAFKKFGKKLAEIETKISERNHDPNLRNRTGPAQLPYTVLLPTSETGLTFRGIPNSISI

SEQ ID NO: 105 - protein, 10 aa, Artificial Sequence
  Artificial Sequence
SSDFLTYGIK

SEQ ID NO: 106 - protein, 8 aa, Artificial Sequence
  Artificial Sequence
GTVVLMPK

SEQ ID NO: 107 - protein, 13 aa, Artificial Sequence
  Artificial Sequence
NVLDFNAITSIGK

SEQ ID NO: 108 - protein, 31 aa, Artificial Sequence
  Artificial Sequence
GGVIDTATGILGQGVSLVGGVIDTATSFLGR

SEQ ID NO: 109 - protein, 19 aa, Artificial Sequence
  Artificial Sequence
IFFVNDTYLPSATPAPLLK

SEQ ID NO: 110 - protein, 8 aa, Artificial Sequence
  Artificial Sequence
DENFGHLK

SEQ ID NO: 111 - protein, 11 aa, Artificial Sequence
  Artificial Sequence
SLSHDVIPLFK

SEQ ID NO: 112 - protein, 8 aa, Artificial Sequence
  Artificial Sequence
SLYEGGIK

SEQ ID NO: 113 - protein, 16 aa, Artificial Sequence
  Artificial Sequence
TDGENVLQFPPPHVAK

SEQ ID NO: 114 - protein, 8 aa, Artificial Sequence
  Artificial Sequence
INSLPTAK

SEQ ID NO: 115 - protein, 6 aa, Artificial Sequence
  Artificial Sequence
TILFLK

SEQ ID NO: 116 - protein, 10 aa, Artificial Sequence
  Artificial Sequence
HLSVLHPIYK

SEQ ID NO: 117 - protein, 12 aa, Artificial Sequence
  Artificial Sequence
QSLINADGIIEK

SEQ ID NO: 118 - protein, 15 aa, Artificial Sequence
  Artificial Sequence
FIPAEGTPEYDEMVK

SEQ ID NO: 119 - protein, 6 aa, Artificial Sequence
  Artificial Sequence
ALEAFK

SEQ ID NO: 120 - protein, 8 aa, Artificial Sequence
  Artificial Sequence
GIPNSISI